(19) United States
(12) Patent Application Publication
(10) Pub. No.: US 2023/0399410 A1
(43) Pub. Date: Dec. 14, 2023

(54) COMPOSITIONS AND METHODS FOR TREATING POLYMYALGIA RHEUMATICA BY ADMINISTERING AN IL-6R ANTAGONIST

(71) Applicants: Sanofi Biotechnology, Gentilly (FR); Regeneron Pharmaceuticals, Inc., Tarrytown, NY (US)

(72) Inventors: Jennifer SLOANE LAZAR, Cambridge, MA (US); Yong LIN, Cambridge, MA (US); Wanling WONG, Cambridge, MA (US); Ying LIU, Cambridge, MA (US); Frédéric MARRACHE, Paris (FR); Kerri FORD, Cambridge, MA (US); Stefano FIORE, Cambridge, MA (US); Lita ARAUJO, Cambridge, MA (US); Danielle ISAMAN, Cambridge, MA (US); Jeffrey CURTIS, Hoover, AL (US); Chad NIVENS, Tarrytown, NY (US); Bola AKINLADE, Tarrytown, NY (US); Angeliki GIANNELOU, Tarrytown, NY (US)

(21) Appl. No.: 18/130,980

(22) Filed: Apr. 5, 2023

Related U.S. Application Data
(60) Provisional application No. 63/447,796, filed on Feb. 23, 2023.
(60) Provisional application No. 63/445,331, filed on Feb. 14, 2023.
(60) Provisional application No. 63/445,329, filed on Feb. 14, 2023.
(60) Provisional application No. 63/424,627, filed on Nov. 11, 2022.
(60) Provisional application No. 63/424,035, filed on Nov. 9, 2022.
(60) Provisional application No. 63/389,317, filed on Jul. 14, 2022.
(60) Provisional application No. 63/350,761, filed on Jun. 9, 2022.
(60) Provisional application No. 63/327,850, filed on Apr. 6, 2022.

(51) Int. Cl. C07K 16/28; A61P 21/00; A61K 31/573; A61K 39/395; A61P 29/00

(52) U.S. Cl. CPC C07K 16/2866; A61P 21/00; A61K 31/573; A61K 39/3955; A61P 29/00; A61K 2039/505

(57) ABSTRACT

The present disclosure relates to the use of an anti-IL6 receptor antibody, or an antigen-binding fragment thereof, for treating polymyalgia rheumatica (PMR).

### RELATED APPLICATIONS

This application claims priority to U.S. Provisional Patent Application Serial Nos.: 63/327,850, filed Apr. 6, 2022; 63/350,761, filed Jun. 9, 2022; 63/389,317, filed Jul. 14, 2022; 63/424,035, filed Nov. 9, 2022; 63/424,627, filed Nov. 11, 2022; 63/445,329, filed Feb. 14, 2023; 63/445,331, filed Feb. 14, 2023; and 63/447,796, filed Feb. 23, 2023. The entire disclosure of each of these applications is hereby incorporated herein by reference in its entirety.

### FIELD

The present disclosure relates to the field of therapeutic treatment of polymyalgia rheumatica (PMR). More specifically, the disclosure relates to the use of interleukin-6 receptor (IL-6R) antagonists, such as anti-IL-6R antibodies to treat polymyalgia rheumatica.

### BACKGROUND

Polymyalgia rheumatica (PMR) is a chronic, inflammatory disease of unknown etiology characterized by pain and morning stiffness of the shoulder, neck and pelvic girdle, and is frequently associated with low-grade fever, fatigue, malaise, and weight loss. The debilitating effect of the disease can significantly affect the quality of life of PMR patients. It typically affects individuals older than 50 years, with prevalence varying by age and population (Gonzalez, G. et al., 2009 Arthritis Rheum. 61(10):1454-61). In 2008, the number of PMR cases in the US was estimated to be 711,000 (Lawrence, R. C. et al., 2008 Arthritis Rheum. 58(1):26-35). The prevalence is higher in women than in men and increases dramatically with age. Based on the only population-based study of PMR in the US, the prevalence ranged from 21 per 100,000 among persons ages 50-54 years to 4,070 per 100,000 among those age ≥90 years (Lawrence, et al. Estimates of the prevalence of arthritis and other rheumatic conditions in the United States, Part II. Arthritis Rheum. 2008; 58(1):26-35). In Europe, higher rates have been noted in Northern European compared with Southern European populations. In patients who are ≥50 years of age, annual incidence rates of 50/100,000 were noted in Sweden and 68/100,000 in Denmark compared to 13/100,000 in Italy and 14-19/100,000 in Spain (Gonzalez, G. et al., 2009 Arthritis Rheum. 61(10):1454-61).

Although the cause is unknown, both genetic and the environmental factors are thought to be involved. Research suggests that inflammation of the joints and the bursae around these joints leads to the symptoms of pain and stiffness associated with PMR. Although there are no definitive tests, there are guidelines to help in the clinical diagnosis of PMR (Dasgupta, B. et al. 2010 Rheumatology. 49(1): 186-90).

Although PMR is typically treated with low doses of corticosteroids (CS), there is a subset of patients who are steroid-dependent or are unable to taper off prednisone or equivalent below 10 mg/day without relapse of symptoms and are therefore at risk of complications of long-term steroid therapy. In the 2015 American College of Rheumatology/European League against Rheumatism (ACR/EULAR) guidelines, the recommended minimum effective starting dose is within the range of 12.5-25 mg of prednisone (or equivalent) daily (Dejaco, C. et al. 2017 Rheumatology. 56(4):506-15). Mean treatment duration is approximately 2 to 3 years. CS with a slow taper will reduce symptoms rapidly for most patients. However, nearly half of the patients with PMR treated with CS do not respond adequately based on laboratory and clinical markers of disease activity in one report (Dasgupta, B. et al. 2010 Rheumatology. 49(1):186-90). In another report, among patients with baseline prednisone doses >10 mg/day, only 30% had remission at 1 year (Caporali, R. et al. 2004 Ann Intern Med. 141(7):493-500).

The current treatment protocol for PMR includes long-term oral glucocorticoid therapy. One drawback of long-term oral glucocorticoid therapy is the potential link to comorbidity. (Chatzigeorgiou C, et al. Comorbidity in polymyalgia rheumatica. Reumatismo. 2018 Mar. 27; 70(1):35-43). There remains a need for an effective treatment for PMR with fewer adverse side effects.

### SUMMARY

In one aspect, method for treating polymyalgia rheumatica (PMR) in a subject in need thereof comprising administering an effective amount of an antibody or antigen-binding fragment thereof that specifically binds IL-6 receptor is provided.

In certain exemplary embodiments, the subject has PMR that is refractory to steroids or is refractory to steroid taper. In certain exemplary embodiments, the subject has had an inadequate response to steroids or cannot tolerate steroid taper.

In certain exemplary embodiments, the steroids comprise corticosteroids. In certain exemplary embodiments, the corticosteroids comprise prednisone. In certain exemplary embodiments, the subject was previously treated with a dose of ≥7.5 mg/day, and/or ≤25 mg/day or ≤20 mg/day, of prednisone.

In certain exemplary embodiments, the antibody or antigen-binding fragment thereof is administered in combination with another therapeutic agent. In certain exemplary embodiments, the therapeutic agent comprises a corticosteroid. In certain exemplary embodiments, the corticosteroid comprises prednisone.

In certain exemplary embodiments, the prednisone is administered at a dose of about 15 mg/day. In certain exemplary embodiments, the dose of prednisone is discontinued or tapered, optionally to <2.5 or 2.0 mg prednisone/day.

In certain exemplary embodiments, the prednisone is discontinued beginning at between about 10 weeks and about 20 weeks after administering a first dose of the antibody. In certain exemplary embodiments, the prednisone is discontinued beginning at about 14 weeks after administering a first dose of the antibody.

In certain exemplary embodiments, the subject was previously treated with a disease modifying antirheumatic drug (cDMARD).

In certain exemplary embodiments, the subject is concomitantly treated with a cDMARD.

In certain exemplary embodiments, the cDMARD is selected from the group consisting of methotrexate, azathioprine, and leflunomide. In certain exemplary embodiments, the cDMARD is methotrexate.

In certain exemplary embodiments, the antibody or antigen-binding fragment thereof is administered at a dose of about 200 mg.

In certain exemplary embodiments, the antibody or antigen-binding fragment thereof is administered at an initial dose of about 200 mg and one or more secondary doses of about 200 mg administered every other week (q2w).

In certain exemplary embodiments, the antibody or antigen-binding fragment thereof comprises heavy chain complementarity determining region (HCDR) sequences of SEQ ID NOs: 3, 4 and 5, and comprises light chain complementarity determining region (LCDR) sequences of SEQ ID NOs: 6, 7 and 8.

In certain exemplary embodiments, the antibody is sarilumab.

In one aspect, a method for treating polymyalgia rheumatica (PMR) in a subject in need thereof comprising administering an effective amount of an antibody or antigen-binding fragment thereof that specifically binds IL-6 receptor, wherein the antibody or antigen-binding fragment thereof comprises heavy chain complementarity determining region (HCDR) sequences of SEQ ID NOs: 3, 4 and 5, and comprises light chain complementarity determining region (LCDR) sequences of SEQ ID NOs: 6, 7 and 8, is provided.

In certain exemplary embodiments, the subject has PMR that is refractory to steroids or is refractory to steroid taper. In certain exemplary embodiments, the steroids comprise corticosteroids. In certain exemplary embodiments, the corticosteroids comprise prednisone.

In certain exemplary embodiments, the subject was previously treated with a dose of ≥7.5 mg/day of prednisone.

In certain exemplary embodiments, the antibody or antigen-binding fragment thereof is administered in combination with another therapeutic agent.

In certain exemplary embodiments, the therapeutic agent comprises a corticosteroid. In certain exemplary embodiments, the corticosteroid comprises prednisone. In certain exemplary embodiments, the prednisone is administered at a dose of about 15 mg/day. In certain exemplary embodiments, the dose of prednisone is tapered.

In certain exemplary embodiments, the antibody or antigen-binding fragment thereof is administered at a dose of about 200 mg.

In certain exemplary embodiments, the antibody or antigen-binding fragment thereof is administered at an initial dose of about 200 mg and one or more secondary doses of about 200 mg administered every other week (q2w).

In certain exemplary embodiments, the subject is at least 50 years old.

In certain exemplary embodiments, the subject has bilateral shoulder pain.

In certain exemplary embodiments, the subject has a C-reactive protein (CRP) level of >10 mg/L and/or an erythrocyte sedimentation rate (ESR)>30 mm/hr.

In certain exemplary embodiments, the subject has morning stiffness.

In certain exemplary embodiments, the subject has an absence of joint involvement other than the shoulder joint.

In certain exemplary embodiments, the subject has hip pain or limited range of motion.

In certain exemplary embodiments, the subject is seronegative for rheumatoid factor (RF) and anti-cyclic citrullinated peptide (anti-CCP).

In certain exemplary embodiments, the subject does not have a disorder selected from the group consisting of giant cell arteritis, rheumatoid arthritis, inflammatory arthritis, connective tissue disease, rhabdomyolysis, neuropathic muscular disease, and active fibromyalgia. In certain exemplary embodiments, the connective tissue disease is selected from the group consisting of systemic lupus erythematosus, systemic sclerosis, vasculitis, myositis, mixed connective tissue disease, and ankylosing spondylitis.

In certain exemplary embodiments, the subject has at least one shoulder with subdeltoid bursitis and/or biceps tenosynovitis and/or posterior or axillary glenohumeral synovitis, and at least one hip with synovitis and/or trochanteric bursitis.

In certain exemplary embodiments, at least one symptom of polymyalgia rheumatica in the subject is improved after administering the antibody or antigen-binding fragment thereof. In certain exemplary embodiments, the symptom is selected from the group consisting of: shoulder pain associated with inflammatory stiffness; hip pain associated with inflammatory stiffness; elevated C-reactive protein (CRP) levels; and elevated erythrocyte sedimentation rate (ESR).

In certain exemplary embodiments, treatment with the antibody or antigen-binding fragment thereof results in the improvement of least one patient reported outcome measure or clinician reported outcome measure selected from the group consisting of: functional assessment of chronic illness therapy fatigue scale (FACIT-Fatigue), EuroQol five-dimensional three-level questionnaire (EQ-5D-3L), and Short form-36v2 (SF-36v2), health assessment questionnaire disability index (HAQ-DI), and physician global assessment of disease activity-Visual Analog Scale (MD-VAS).

In certain exemplary embodiments, treatment with the antibody or antigen-binding fragment thereof results in a decrease in glucocorticoid toxicity index (GTI) score.

In certain exemplary embodiments, treatment with the antibody or antigen-binding fragment thereof results in a decrease in PMR activity score (PMR-AS). In certain exemplary embodiments, the decrease in PMR-AS is at least 3 points. In certain exemplary embodiments, the decrease in PMR-AS is at least 5 points.

In certain exemplary embodiments, treatment with the antibody or antigen-binding fragment thereof results in an increase in time to first PMR flare. In certain exemplary embodiments, the symptoms of PMR flare are selected from the group consisting of shoulder pain associated with inflammatory stiffness and hip girdle pain associated with inflammatory stiffness.

In certain exemplary embodiments, the antibody or antigen-binding fragment thereof is administered for at least 12 weeks. In certain exemplary embodiments, the antibody or antigen-binding fragment thereof is administered for at least 52 weeks.

In certain exemplary embodiments, treatment with the antibody or antigen-binding fragment thereof results in remission of PMR in the subject.

In certain exemplary embodiments, the subject has an absence of disease flare in remission. In certain exemplary embodiments, the symptoms of PMR flare are selected from the group consisting of shoulder pain associated with inflammatory stiffness and hip girdle pain associated with inflammatory stiffness. In certain exemplary embodiments, the remission is achieved at week 12 after starting treatment with the antibody or antigen-binding fragment thereof. In certain exemplary embodiments, the remission is sustained at week 52 after starting treatment with the antibody or antigen-binding fragment thereof. In certain exemplary embodiments, the remission is achieved at week 12 and sustained to week 52 after starting treatment with the antibody or antigen-binding fragment thereof. In still other exemplary embodiments, the remission (e.g., absence of disease flare) is achieved at week 16 and sustained to week 52 or is achieved at week 24 and sustained to week 52 after starting treatment with the antibody or antigen-binding fragment thereof.

In certain exemplary embodiments, the antibody or antigen-binding fragment thereof comprises a heavy chain variable region of sequence SEQ ID NO: 1 and the light chain variable region sequence of SEQ ID NO: 2.

In certain exemplary embodiments, the antibody or antigen-binding fragment thereof comprises a heavy chain comprising SEQ ID NO:9 and a light chain comprising SEQ ID NO:10.

In certain exemplary embodiments, the antibody is sarilumab.

In certain exemplary embodiments, the antibody or antigen-binding fragment thereof is administered subcutaneously. In certain exemplary embodiments, the pharmaceutical composition is administered subcutaneously using a needle and syringe, a pen delivery device, or an autoinjector.

In certain exemplary embodiments, the antibody is administered using a prefilled syringe containing about 175 mg/mL sarilumab.

In another aspect, a method for treating polymyalgia rheumatica (PMR) in a subject in need thereof comprising administering to the subject a single initial dose of an antibody or antigen-binding fragment thereof that specifically binds IL-6 receptor, followed by one or more secondary doses of the antibody or antigen-binding fragment thereof, wherein the antibody or antigen-binding fragment thereof comprises heavy chain complementarity determining region (HCDR) sequences of SEQ ID NOs: 3, 4 and 5, and comprises light chain complementarity determining region (LCDR) sequences of SEQ ID NOs: 6, 7 and 8, is provided.

In certain exemplary embodiments, the subject has PMR that is refractory to steroids or is refractory to steroid taper. In certain exemplary embodiments, the steroids comprise corticosteroids. In certain exemplary embodiments, the corticosteroids comprise prednisone.

In certain exemplary embodiments, the subject was previously treated with a dose of ≥7.5 mg/day of prednisone.

In certain exemplary embodiments, the antibody or antigen-binding fragment thereof is administered in combination with another therapeutic agent. In certain exemplary embodiments, wherein the therapeutic agent comprises a corticosteroid. In certain exemplary embodiments, the corticosteroid comprises prednisone. In certain exemplary embodiments, the prednisone is administered at a dose of about 15 mg/day. In certain exemplary embodiments, the dose of prednisone is tapered.

In certain exemplary embodiments, the initial and secondary doses of the antibody or antigen-binding fragment thereof are each about 200 mg.

In certain exemplary embodiments, the secondary doses are administered every other week (q2w).

In certain exemplary embodiments, the subject is at least 50 years old.

In certain exemplary embodiments, the subject has bilateral shoulder pain.

In certain exemplary embodiments, the subject has a C-reactive protein (CRP) level of ≥10 mg/L and/or an erythrocyte sedimentation rate (ESR)>30 mm/hr.

In certain exemplary embodiments, the subject has morning stiffness.

In certain exemplary embodiments, the subject has an absence of joint involvement other than the shoulder joint.

In certain exemplary embodiments, the subject has hip pain or limited range of motion.

In certain exemplary embodiments, the subject is seronegative for rheumatoid factor (RF) and anti-cyclic citrullinated peptide (anti-CCP).

In certain exemplary embodiments, the subject has at least one shoulder with subdeltoid bursitis and/or biceps tenosynovitis and/or posterior or axillary glenohumeral synovitis, and at least one hip with synovitis and/or trochanteric bursitis.

In certain exemplary embodiments, at least one symptom of polymyalgia rheumatica in the subject is improved after administering the antibody or antigen-binding fragment thereof. In certain exemplary embodiments, the symptom is selected from the group consisting of: shoulder pain associated with inflammatory stiffness; hip pain associated with inflammatory stiffness; elevated C-reactive protein (CRP) levels; and elevated erythrocyte sedimentation rate (ESR).

In certain exemplary embodiments, treatment with the antibody or antigen-binding fragment thereof results in the improvement of least one patient reported outcome measure or clinician reported outcome measure selected from the group consisting of: functional assessment of chronic illness therapy fatigue scale (FACIT-Fatigue), EuroQol five-dimensional three-level questionnaire (EQ-5D-3L), and Short form-36v2 (SF-36v2), health assessment questionnaire disability index (HAQ-DI), and physician global assessment of disease activity-Visual Analog Scale (MD-VAS).

In certain exemplary embodiments, treatment with the antibody or antigen-binding fragment thereof results in a decrease in glucocorticoid toxicity index (GTI) score.

In certain exemplary embodiments, treatment with the antibody or antigen-binding fragment thereof results in a decrease in PMR activity score (PMR-AS). In certain exemplary embodiments, the decrease in PMR-AS is at least 3 points. In certain exemplary embodiments, the decrease in PMR-AS is at least 5 points.

In certain exemplary embodiments, treatment with the antibody or antigen-binding fragment thereof results in an increase in time to first PMR flare. In certain exemplary embodiments, the symptoms of PMR flare are selected from the group consisting of shoulder pain associated with inflammatory stiffness and hip girdle pain associated with inflammatory stiffness.

In certain exemplary embodiments, the antibody or antigen-binding fragment thereof is administered for at least 12 weeks. In certain exemplary embodiments, the antibody or antigen-binding fragment thereof is administered for at least 52 weeks.

In certain exemplary embodiments, treatment with the antibody or antigen-binding fragment thereof results in remission of PMR in the subject.

In certain exemplary embodiments, the subject has an absence of disease flare in remission. In certain exemplary embodiments, the symptoms of PMR flare are selected from the group consisting of shoulder pain associated with inflammatory stiffness and hip girdle pain associated with inflammatory stiffness. In certain exemplary embodiments, the remission is achieved at week 12 after starting treatment with the antibody or antigen-binding fragment thereof. In certain exemplary embodiments, the remission is sustained at week 16 after starting treatment with the antibody or antigen-binding fragment thereof. In certain exemplary embodiments, the remission is sustained at week 24 after starting treatment with the antibody or antigen-binding fragment thereof. In certain exemplary embodiments, the remission is sustained at week 52 after starting treatment with the antibody or antigen-binding fragment thereof.

In certain exemplary embodiments, the antibody or antigen-binding fragment thereof comprises a heavy chain variable region of sequence SEQ ID NO: 1 and the light chain variable region sequence of SEQ ID NO: 2.

In certain exemplary embodiments, the antibody or antigen-binding fragment thereof comprises a heavy chain comprising SEQ ID NO:9 and a light chain comprising SEQ ID NO:10.

In certain exemplary embodiments, the antibody is sarilumab.

In certain exemplary embodiments, the antibody or antigen-binding fragment thereof is administered subcutaneously. In certain exemplary embodiments, the pharmaceutical composition is administered subcutaneously using a needle and syringe, a pen delivery device, or an autoinjector.

In certain exemplary embodiments, the antibody is administered using a prefilled syringe containing about 175 mg/mL sarilumab.

In another aspect, a method of reducing or eliminating the dependence of a subject with polymyalgia rheumatica (PMR) on a background therapy comprising corticosteroids for the treatment of PMR comprising: (a) selecting a patient who has PMR that is partially controlled or uncontrolled with a background therapy comprising corticosteroids; (b) administering to the patient a defined dose of a therapeutically effective amount of an antibody or antigen-binding fragment thereof that specifically binds IL-6 receptor at a defined frequency for an initial treatment period while maintaining the subject's background PMR therapy for the initial treatment period; and (c) gradually reducing or eliminating the dosage of corticosteroids administered to the subject over the course of a subsequent treatment period while continuing to administer the antibody or antigen-binding fragment thereof to the subject at the defined frequency and dose used during the initial treatment period, is provided.

In certain exemplary embodiments, the antibody or antigen binding fragment thereof comprises heavy chain complementarity determining region (HCDR) sequences of SEQ ID NOs: 3, 4 and 5, and comprises light chain complementarity determining region (LCDR) sequences of SEQ ID NOs: 6, 7 and 8.

In certain exemplary embodiments, the corticosteroids comprise prednisone.

In certain exemplary embodiments, the initial dose of prednisone is about 15 mg/day.

In certain exemplary embodiments, the subsequent treatment period is at least 14 weeks. In certain exemplary embodiments, the subsequent treatment period is at least 52 weeks.

In certain exemplary embodiments, the antibody or antigen-binding fragment thereof is administered at a dose of about 200 mg.

In certain exemplary embodiments, the antibody or antigen-binding fragment thereof is administered every other week (q2w).

In certain exemplary embodiments, the subject is at least 50 years old.

In certain exemplary embodiments, the subject has bilateral shoulder pain.

In certain exemplary embodiments, the subject has a C-reactive protein (CRP) level of >10 mg/L and/or an erythrocyte sedimentation rate (ESR)>30 mm/hr.

In certain exemplary embodiments, the subject has morning stiffness.

In certain exemplary embodiments, the subject has an absence of joint involvement other than the shoulder joint.

In certain exemplary embodiments, the subject has hip pain or limited range of motion.

In certain exemplary embodiments, the subject is seronegative for rheumatoid factor (RF) and anti-cyclic citrullinated peptide (anti-CCP).

In certain exemplary embodiments, the subject has at least one shoulder with subdeltoid bursitis and/or biceps tenosynovitis and/or posterior or axillary glenohumeral synovitis, and at least one hip with synovitis and/or trochanteric bursitis.

In certain exemplary embodiments, at least one symptom of polymyalgia rheumatica in the subject is improved after administering the antibody or antigen-binding fragment thereof. In certain exemplary embodiments, the symptom is selected from the group consisting of: shoulder pain associated with inflammatory stiffness; hip pain associated with inflammatory stiffness; elevated C-reactive protein (CRP) levels; and elevated erythrocyte sedimentation rate (ESR).

In certain exemplary embodiments, treatment with the antibody or antigen-binding fragment thereof results in the improvement of least one patient reported outcome measure or clinician reported outcome measure selected from the group consisting of: functional assessment of chronic illness therapy fatigue scale (FACIT-Fatigue), EuroQol five-dimensional three-level questionnaire (EQ-5D-3L), and Short form-36v2 (SF-36v2), health assessment questionnaire disability index (HAQ-DI), and physician global assessment of disease activity-Visual Analog Scale (MD-VAS).

In certain exemplary embodiments, treatment with the antibody or antigen-binding fragment thereof results in a decrease in glucocorticoid toxicity index (GTI) score.

In certain exemplary embodiments, treatment with the antibody or antigen-binding fragment thereof results in a decrease in PMR activity score (PMR-AS). In certain exemplary embodiments, the decrease in PMR-AS is at least 3 points. In certain exemplary embodiments, the decrease in PMR-AS is at least 5 points.

In certain exemplary embodiments, treatment with the antibody or antigen-binding fragment thereof results in an increase in time to first PMR flare. In certain exemplary embodiments, the symptoms of PMR flare are selected from the group consisting of shoulder pain associated with inflammatory stiffness and hip girdle pain associated with inflammatory stiffness.

In certain exemplary embodiments, the antibody or antigen-binding fragment thereof is administered for at least 12 weeks. In certain exemplary embodiments, the antibody or antigen-binding fragment thereof is administered for at least 52 weeks.

In certain exemplary embodiments, treatment with the antibody or antigen-binding fragment thereof results in remission of PMR in the subject.

In certain exemplary embodiments, the subject has an absence of disease flare in remission. In certain exemplary embodiments, the symptoms of PMR flare are selected from the group consisting of shoulder pain associated with inflammatory stiffness and hip girdle pain associated with inflammatory stiffness. In certain exemplary embodiments, the remission is achieved at week 12 after starting treatment with the antibody or antigen-binding fragment thereof. In certain exemplary embodiments, the remission is sustained at week 52 after starting treatment with the antibody or antigen-binding fragment thereof. In certain exemplary embodiments, the remission (e.g., absence of disease flare) is achieved at week 12 and sustained to week 52 after starting treatment with the antibody or antigen-binding fragment thereof. In still other exemplary embodiments, the remission (e.g., absence of disease flare) is achieved at week 16 and sustained to week 52 or is achieved at week 24 and sustained to week 52 after starting treatment with the antibody or antigen-binding fragment thereof.

In certain exemplary embodiments, the subject treated with the antibody obtains an increased resolution of PMR signs and symptoms or GC-free resolution of PMR signs and symptoms. In certain exemplary embodiments, the resolution of PMR signs and symptoms was achieved from week 4 after starting treatment with the antibody or antigen-binding fragment thereof. In certain exemplary embodiments, the GC-free resolution of PMR signs and symptoms was maintained from week 16 after starting treatment with the antibody or antigen-binding fragment thereof to week 52. In certain exemplary embodiments, the signs and symptoms can include morning stiffness and/or pain, in the neck, shoulder and/or hip girdles; limited range of motion of the shoulders and/or hip girdles; constitutional symptoms, such as fatigue, weight loss and low-grade fever; and other features judged to by the clinician-investigator to be consistent with PMR.

In certain exemplary embodiments, the antibody or antigen-binding fragment thereof comprises a heavy chain variable region of sequence SEQ ID NO: 1 and the light chain variable region sequence of SEQ ID NO: 2.

In certain exemplary embodiments, the antibody or antigen-binding fragment thereof comprises a heavy chain comprising SEQ ID NO:9 and a light chain comprising SEQ ID NO:10.

In certain exemplary embodiments, the antibody is sarilumab.

In certain exemplary embodiments, the antibody or antigen-binding fragment thereof is administered subcutaneously. In certain exemplary embodiments, the pharmaceutical composition is administered subcutaneously using a needle and syringe, a pen delivery device, or an autoinjector.

In certain exemplary embodiments, the antibody is administered using a prefilled syringe containing about 175 mg/mL sarilumab.

In another aspect, a method for treating polymyalgia rheumatica (PMR) in a subject in need thereof is provided, comprising administering an effective amount of an antibody or antigen-binding fragment thereof that specifically binds IL-6 receptor, wherein the antibody or antigen-binding fragment thereof comprises heavy chain complementarity determining region (HCDR) sequences of SEQ ID NOs: 3, 4 and 5, and comprises light chain complementarity determining region (LCDR) sequences of SEQ ID NOs: 6, 7 and 8, and wherein the subject has had an inadequate response to steroids.

In certain exemplary embodiments, the steroids comprise corticosteroids, e.g., prednisone.

In certain exemplary embodiments, the subject is an adult.

In certain exemplary embodiments, the antibody or antigen-binding fragment thereof comprises a heavy chain variable region of sequence SEQ ID NO: 1 and the light chain variable region sequence of SEQ ID NO: 2.

In certain exemplary embodiments, the antibody or antigen-binding fragment thereof comprises a heavy chain comprising SEQ ID NO:9 and a light chain comprising SEQ ID NO:10.

In certain exemplary embodiments, the antibody is sarilumab.

In certain exemplary embodiments, the antibody or antigen-binding fragment thereof is administered subcutaneously. In certain exemplary embodiments, the pharmaceutical composition is administered subcutaneously using a needle and syringe, a pen delivery device, or an autoinjector.

In certain exemplary embodiments, the antibody is administered using a prefilled syringe containing about 175 mg/mL sarilumab.

In another aspect, a method for treating polymyalgia rheumatica (PMR) in a subject in need thereof is provided, comprising administering an effective amount of an antibody or antigen-binding fragment thereof that specifically binds IL-6 receptor, wherein the antibody or antigen-binding fragment thereof comprises heavy chain complementarity determining region (HCDR) sequences of SEQ ID NOs: 3, 4 and 5, and comprises light chain complementarity determining region (LCDR) sequences of SEQ ID NOs: 6, 7 and 8, and wherein the subject cannot tolerate steroid taper.

In certain exemplary embodiments, the steroids comprise corticosteroids, e.g., prednisone.

In certain exemplary embodiments, the subject is an adult.

In certain exemplary embodiments, the antibody or antigen-binding fragment thereof comprises a heavy chain variable region of sequence SEQ ID NO: 1 and the light chain variable region sequence of SEQ ID NO: 2.

In certain exemplary embodiments, the antibody or antigen-binding fragment thereof comprises a heavy chain comprising SEQ ID NO:9 and a light chain comprising SEQ ID NO:10.

In certain exemplary embodiments, the antibody is sarilumab.

In certain exemplary embodiments, the antibody or antigen-binding fragment thereof is administered subcutaneously. In certain exemplary embodiments, the pharmaceutical composition is administered subcutaneously using a needle and syringe, a pen delivery device, or an autoinjector.

In certain exemplary embodiments, the antibody is administered using a prefilled syringe containing about 175 mg/mL sarilumab.

In another aspect, a method for treating polymyalgia rheumatica (PMR) in a subject in need thereof is provided, comprising administering an effective amount of an antibody or antigen-binding fragment thereof that specifically binds IL-6 receptor, wherein the antibody or antigen-binding fragment thereof comprises heavy chain complementarity determining region (HCDR) sequences of SEQ ID NOs: 3, 4 and 5, and comprises light chain complementarity determining region (LCDR) sequences of SEQ ID NOs: 6, 7 and 8, and wherein the subject has had an inadequate response to steroids or wherein the subject cannot tolerate steroid taper.

In certain exemplary embodiments, the steroids comprise corticosteroids, e.g., prednisone.

In certain exemplary embodiments, the subject is an adult.

In another aspect, a method for treating polymyalgia rheumatica (PMR) in an adult subject in need thereof is provided, comprising administering an effective amount of an antibody or antigen-binding fragment thereof that specifically binds IL-6 receptor, wherein the antibody or antigen-binding fragment thereof comprises heavy chain complementarity determining region (HCDR) sequences of SEQ ID NOs: 3, 4 and 5, and comprises light chain complementarity determining region (LCDR) sequences of SEQ ID NOs: 6, 7 and 8, and wherein the subject has had an inadequate response to corticosteroids or wherein the subject cannot tolerate corticosteroid taper.

In certain exemplary embodiments, the antibody or antigen-binding fragment thereof comprises a heavy chain variable region of sequence SEQ ID NO: 1 and the light chain variable region sequence of SEQ ID NO: 2.

In certain exemplary embodiments, the antibody or antigen-binding fragment thereof comprises a heavy chain comprising SEQ ID NO:9 and a light chain comprising SEQ ID NO:10.

In certain exemplary embodiments, the antibody is sarilumab.

In another aspect, a method for treating polymyalgia rheumatica (PMR) in an adult subject in need thereof is provided, comprising administering an effective amount of sarilumab, wherein the patient has had an inadequate response to corticosteroids or wherein the patient cannot tolerate corticosteroid taper.

In certain exemplary embodiments, the antibody or antigen-binding fragment thereof is administered subcutaneously. In certain exemplary embodiments, the pharmaceutical composition is administered subcutaneously using a needle and syringe, a pen delivery device, or an autoinjector.

In certain exemplary embodiments, the antibody is administered using a prefilled syringe containing about 175 mg/mL sarilumab.

In another aspect, a method for treating polymyalgia rheumatica (PMR) in a subject in need thereof is provided, comprising administering to the subject a single initial dose of an antibody or antigen-binding fragment thereof that specifically binds IL-6 receptor, followed by one or more secondary doses of the antibody or antigen-binding fragment thereof, wherein the antibody or antigen-binding fragment thereof comprises heavy chain complementarity determining region (HCDR) sequences of SEQ ID NOs: 3, 4 and 5, and comprises light chain complementarity determining region (LCDR) sequences of SEQ ID NOs: 6, 7 and 8, and wherein the subject has had an inadequate response to steroids.

In certain exemplary embodiments, the steroids comprise corticosteroids, e.g., prednisone.

In certain exemplary embodiments, the subject is an adult.

In certain exemplary embodiments, the antibody or antigen-binding fragment thereof comprises a heavy chain variable region of sequence SEQ ID NO: 1 and the light chain variable region sequence of SEQ ID NO: 2.

In certain exemplary embodiments, the antibody or antigen-binding fragment thereof comprises a heavy chain comprising SEQ ID NO:9 and a light chain comprising SEQ ID NO:10.

In certain exemplary embodiments, the antibody is sarilumab.

In certain exemplary embodiments, the antibody or antigen-binding fragment thereof is administered subcutaneously. In certain exemplary embodiments, the pharmaceutical composition is administered subcutaneously using a needle and syringe, a pen delivery device, or an autoinjector.

In certain exemplary embodiments, the antibody is administered using a prefilled syringe containing about 175 mg/mL sarilumab.

In another aspect, a method for treating polymyalgia rheumatica (PMR) in a subject in need thereof is provided, comprising administering to the subject a single initial dose of an antibody or antigen-binding fragment thereof that specifically binds IL-6 receptor, followed by one or more secondary doses of the antibody or antigen-binding fragment thereof, wherein the antibody or antigen-binding fragment thereof comprises heavy chain complementarity determining region (HCDR) sequences of SEQ ID NOs: 3, 4 and 5, and comprises light chain complementarity determining region (LCDR) sequences of SEQ ID NOs: 6, 7 and 8, and wherein the subject cannot tolerate steroid taper.

In certain exemplary embodiments, the steroids comprise corticosteroids, e.g., prednisone.

In certain exemplary embodiments, the subject is an adult.

In certain exemplary embodiments, the antibody or antigen-binding fragment thereof comprises a heavy chain variable region of sequence SEQ ID NO: 1 and the light chain variable region sequence of SEQ ID NO: 2.

In certain exemplary embodiments, the antibody or antigen-binding fragment thereof comprises a heavy chain comprising SEQ ID NO:9 and a light chain comprising SEQ ID NO:10.

In certain exemplary embodiments, the antibody is sarilumab.

In certain exemplary embodiments, the antibody or antigen-binding fragment thereof is administered subcutaneously. In certain exemplary embodiments, the pharmaceutical composition is administered subcutaneously using a needle and syringe, a pen delivery device, or an autoinjector.

In certain exemplary embodiments, the antibody is administered using a prefilled syringe containing about 175 mg/mL sarilumab.

In another aspect, a method for treating polymyalgia rheumatica (PMR) in a subject in need thereof is provided, comprising administering to the subject a single initial dose of an antibody or antigen-binding fragment thereof that specifically binds IL-6 receptor, followed by one or more secondary doses of the antibody or antigen-binding fragment thereof, wherein the antibody or antigen-binding fragment thereof comprises heavy chain complementarity determining region (HCDR) sequences of SEQ ID NOs: 3, 4 and 5, and comprises light chain complementarity determining region (LCDR) sequences of SEQ ID NOs: 6, 7 and 8, and wherein the subject has had an inadequate response to steroids or wherein the subject cannot tolerate steroid taper.

In certain exemplary embodiments, the steroids comprise corticosteroids, e.g., prednisone.

In certain exemplary embodiments, the subject is an adult.

In certain exemplary embodiments, the antibody or antigen-binding fragment thereof comprises a heavy chain variable region of sequence SEQ ID NO: 1 and the light chain variable region sequence of SEQ ID NO: 2.

In certain exemplary embodiments, the antibody or antigen-binding fragment thereof comprises a heavy chain comprising SEQ ID NO:9 and a light chain comprising SEQ ID NO:10.

In certain exemplary embodiments, the antibody is sarilumab.

In certain exemplary embodiments, the antibody or antigen-binding fragment thereof is administered subcutaneously. In certain exemplary embodiments, the pharmaceutical composition is administered subcutaneously using a needle and syringe, a pen delivery device, or an autoinjector.

In certain exemplary embodiments, the antibody is administered using a prefilled syringe containing about 175 mg/mL sarilumab.

In another aspect, a method for treating polymyalgia rheumatica (PMR) in a subject in need thereof is provided, comprising administering an effective amount of an antibody or antigen-binding fragment thereof that specifically binds IL-6 receptor, wherein the antibody or antigen-binding fragment thereof comprises heavy chain complementarity determining region (HCDR) sequences of SEQ ID NOs: 3, 4 and 5, and comprises light chain complementarity determining region (LCDR) sequences of SEQ ID NOs: 6, 7 and 8, or wherein the antibody or antigen-binding fragment thereof comprises heavy chain complementarity determining region (HCDR) sequences of SEQ ID NOs: 17, 18 and 19, and comprises light chain complementarity determining region (LCDR) sequences of SEQ ID NOs: 14, 15 and 16.

In certain exemplary embodiments, the antibody or antigen-binding fragment thereof comprises a heavy chain variable region of sequence SEQ ID NO: 1 and the light chain variable region sequence of SEQ ID NO: 2.

In certain exemplary embodiments, the antibody or antigen-binding fragment thereof comprises a heavy chain comprising SEQ ID NO: 9 and a light chain comprising SEQ ID NO: 10. In certain exemplary embodiments, the antibody is sarilumab.

In certain exemplary embodiments, the antibody or antigen-binding fragment thereof comprises the heavy chain complementarity determining regions (HCDRs) and/or the light chain complementarity determining regions (LCDRs) of an HCVR comprising the amino acid sequence of SEQ ID NO: 13 and the light chain complementarity determining regions (LCDRs) of an LCVR comprising the amino acid sequence of SEQ ID NO: 12.

In certain exemplary embodiments, the antibody or antigen-binding fragment thereof comprises a heavy chain comprising the amino acid sequence of SEQ ID NO: 13 and a light chain comprising the amino acid sequence of SEQ ID NO: 12.

In certain exemplary embodiments, the antibody or antigen-binding fragment thereof is tocilizumab.

In certain exemplary embodiments, the antibody or antigen-binding fragment thereof is administered subcutaneously or intravenously. In certain exemplary embodiments, the pharmaceutical composition is administered subcutaneously using a needle and syringe, a pen delivery device, or an autoinjector.

In certain exemplary embodiments, the antibody or antigen-binding fragment thereof is administered in combination with another therapeutic agent. In certain exemplary embodiments, the therapeutic agent comprises a corticosteroid. In certain exemplary embodiments, the corticosteroid a glucocorticoid. In certain exemplary embodiments, the glucocorticoid is prednisone or an equivalent.

In certain exemplary embodiments, the dose of the glucocorticoid tapered. In certain exemplary embodiments, the subject is able to reduce total glucocorticoid exposure. In certain exemplary embodiments, the subject is able to achieve a minimal glucocorticoid dose of less than or equal to 2.5 mg prednisone or an equivalent per day. In certain exemplary embodiments, the subject is able to achieve a minimal glucocorticoid dose of less than or equal to 2.0 mg prednisone or an equivalent per day.

In certain exemplary embodiments, use of the antibody or antigen-binding fragment thereof leads to a greater reduction in glucocorticoid exposure than use of a conventional immunomodulatory (cIM) therapy.

In certain exemplary embodiments, the subject is able to discontinue glucocorticoid therapy. In certain exemplary embodiments, discontinuation of glucocorticoid therapy is defined as a gap in glucocorticoid use of greater than 60 days.

In certain exemplary embodiments, the subject's time to non-persistence is longer with the antibody or antigen-binding fragment thereof than with a conventional immunomodulatory (cIM) therapy.

In certain exemplary embodiments, the antibody or antigen-binding fragment thereof is a second line treatment. In certain exemplary embodiments, the antibody or antigen-binding fragment thereof is a third line treatment.

In certain exemplary embodiments, the method comprises administering to the subject a single initial dose of an antibody or antigen-binding fragment thereof that specifically binds IL-6 receptor, followed by one or more secondary doses of the antibody or antigen-binding fragment thereof.

In another aspect, a method of reducing or eliminating the dependence of a subject with polymyalgia rheumatica (PMR) on a background therapy comprising glucocorticoids for the treatment of PMR is provided comprising (a) selecting a patient who has PMR that is partially controlled or uncontrolled with a background therapy comprising glucocorticoids, (b) administering to the patient a defined dose of a therapeutically effective amount of an antibody or antigen-binding fragment thereof that specifically binds IL-6 receptor at a defined frequency for an initial treatment period while maintaining the subject's background PMR therapy for the initial treatment period, wherein the antibody or antigen-binding fragment thereof comprises heavy chain complementarity determining region (HCDR) sequences of SEQ ID NOs: 3, 4 and 5, and comprises light chain complementarity determining region (LCDR) sequences of SEQ ID NOs: 6, 7 and 8, or wherein the antibody or antigen-binding fragment thereof comprises heavy chain complementarity determining region (HCDR) sequences of SEQ ID NOs: 17, 18 and 19, and comprises light chain complementarity determining region (LCDR) sequences of SEQ ID NOs: 14, 15 and 16, and (c) gradually reducing or eliminating the dosage of glucocorticoids administered to the subject over the course of a subsequent treatment period while continuing to administer the antibody or antigen-binding fragment thereof to the subject at the defined frequency and dose used during the initial treatment period.

In certain exemplary embodiments, the glucocorticoids comprise prednisone or an equivalent.

In certain exemplary embodiments, at least one symptom of polymyalgia rheumatica in the subject is improved after administering the antibody or antigen-binding fragment thereof. In certain exemplary embodiments, the symptom is selected from the group consisting of: shoulder pain associated with inflammatory stiffness; hip pain associated with inflammatory stiffness; elevated C-reactive protein (CRP) levels; and elevated erythrocyte sedimentation rate (ESR).

### BRIEF DESCRIPTION OF THE FIGURES

The foregoing and other features and advantages of the disclosure will be more fully understood from the following detailed description of illustrative embodiments taken in conjunction with the accompanying drawings. The file of this patent contains at least one drawing/photograph executed in color. Copies of this patent with color drawing(s)/photograph(s) will be provided by the Office upon request and payment of the necessary fee.

FIG. 1 schematically depicts the overview of the study design of Example 1. The study was a randomized, double-blind, placebo-controlled study to evaluate the efficacy and safety of sarilumab in patients with polymyalgia rheumatica. The study included two groups of patients with active PMR. Participants in group 1 received sarilumab 200 mg q2w with a 14-week taper of corticosteroids. Participants in group 2 received sarilumab matching placebo q2w with a 52-week taper of corticosteroids. All patients received sarilumab 200 mg or placebo for 52-weeks.

FIGS. 2A-2B depict the questionnaire for the Eurow1-5 dimensions 3-level version (EQ-5D-3L) assessment, a generic PRO instrument which measures health status.

FIGS. 3A-3C depict the questionnaire for the Short Form 36v2 (SF-36v2), a short-form generic, 36-item PRO instrument that evaluates 8 multi-item dimensions of health: physical functioning, social functioning, role limitations due to physical problems, role limitations due to emotional problems, mental health, energy/vitality, bodily pain, and general health perception.

FIG. 4 depicts the questionnaire for the physician global assessment of disease activity-Visual Analog Scale [MD-VAS] used to rate the patient's disease activity on an anchored 100 mm horizontal VAS where 0 is considered not active and 100 is considered the most active.

FIG. 5 depicts a forest plot for subgroup analysis of the proportion of patients achieving sustained remission at week 52. Subgroup analyses that were performed to assess the consistency in treatment effects. Subgroup analysis of the primary endpoint results demonstrated a numerical trend in favor of the sarilumab 200 mg q2w+14-week taper group compared to the placebo+52-week taper group except in participants with a baseline weight <60 kg; however, the sample size was very small in this subgroup.

FIG. 6 depicts a Kaplan-Meier plot for time to first PMR flare after clinical remission up to 52 weeks for the ITT population. Participants in the sarilumab 200 mg q2w+14-week taper group were less likely to have a PMR flare after achieving clinical remission compared to participants in the placebo+52-week taper group (16.7% versus 29.3%) with a hazard ratio of (95% CI:0.35 to 0.90; p=0.0153).

FIG. 7 depicts a plot of mean change from baseline in PMR activity score over time for the ITT population. The greatest reduction in PMR activity for both the sarilumab 200 mg q2w+14-week taper group and the placebo+52-week taper groups was seen from baseline to Week 12 with reduction in the sarilumab 200 mg q2w+14-week taper group continuing until Week 52.

FIG. 8 graphically depicts SF-36 least-squares mean (LSM) physical component summary scores (PCS) and mental component summary scores (MCS). LSM difference in PCS in the sarilumab 200 mg q2w+14-week taper group versus the placebo+52-week taper group was statistically significant (4.784, 95% CI: 0.865, 8.703; p=0.0172). The LSM difference of MCS scores in the sarilumab 200 mg q2w+14-week taper group versus the placebo+52-week taper group was statistically significant (4.748, 95% CI: 0.484, 9.013; p=0.0295).

FIG. 9 graphically depicts the statistical significance of the improvement from baseline in various SF-36 domains with sarilumab versus placebo. The SF-36 domains include physical function (PF), role physical (RP), bodily pain (BP), general health (GH), vitality (VT), social function (SF), role emotional (RE), and mental health (MH). As shown in FIG. 9, 5 out of 8 domains of SF-36 showed statistically significantly greater improvement with sarilumab versus placebo (MH, RP, BP, SF, and VT).

FIG. 10 graphically depicts the percent of patients reporting an improvement greater than or equal to minimal clinically important difference (MCID) at week 52 for treatment with sarilumab and placebo for various SF-36 metrics including PCS, MCS, PF, RP, BP, GH, VT, SF, RE, and MH. MCID (improvement from baseline) was 2.5 for PCS and MCS. MCID was 5.0 for individual SF-36 domains. These results show that more patients receiving sarilumab reported improvements of greater than or equal to MCID at a statistically significant level for PCS (P=0.0161) and 5 of 8 SF-36 domain scores.

FIG. 11 graphically depicts the percent of patients treated with sarilumab and placebo that reported scores greater than or equal to normative values at baseline and at week 52 for various SF-36 metrics including PCS, MCS, PF, RP, BP, GH, VT, SF, RE, and MH. The threshold values were as follows: PCS and MCS ≥50.0; PF≥66.0, RP≥69.2, BP≥66.4, GH≥66.1, VT≥58.8, SF≥82.1, RE 81.9, and MH≥77.8. These results show that numerically more patients receiving sarilumab reported scores greater than or equal to normative values for SF-36 MCS and for 4 SF-36 domain scores.

FIG. 12 graphically depicts the percent of patients treated with sarilumab and placebo that reported an improvement greater than or equal to MCID for FACIT-F score at week 52. MCID for FACIT-F was an improvement greater than or equal to 4.0. These results show that sarilumab treatment led to numerically greater improvements in FACIT-F score than treatment with placebo (with a higher score representing better functioning or less fatigue).

FIG. 13 graphically depicts the percent of patients treated with sarilumab and placebo that reported FACIT-F scores greater than or equal to normative values at baseline and at week 52. The threshold for normative values was greater than or equal to 43.5. These results show that sarilumab treatment led to numerically greater improvements in FACIT-F score than treatment with placebo.

FIG. 14 graphically depicts the percent of patients treated with sarilumab and placebo that reported an improvement greater than or equal to MCID for HAQ-DI score at week 52. MCID for HAQ-DI was an improvement greater than or equal to 0.22. These results show that sarilumab treatment led to numerically greater improvements in HAQ-DI score than treatment with placebo (with a lower HAQ-DI score representing an improvement).

FIG. 15 graphically depicts the percentage of patients treated with sarilumab and placebo that reported HAQ-DI scores greater than or equal to normative values at baseline and at week 52. The threshold for normative values was less than or equal to 0.25. These results show that sarilumab treatment led to numerically greater improvements in HAQ-DI score than treatment with placebo (with a lower HAQ-DI score representing an improvement).

FIG. 16 graphically depicts the LSM change from baseline at week 52 for Patient Global Assessment of Disease Activity (PtGA) score for patients treated with sarilumab and placebo. These results show that treatment with sarilumab led to numerically greater improvements in PtGA scores versus treatment with placebo (with a higher score representing a higher level of disease activity or a worse global health).

FIG. 17 graphically depicts the percent of patients reporting improvements greater than MCID at week 52 in PtGA score with treatment with sarilumab and placebo. MCID was an improvement of greater than or equal to 10.0. These results show that treatment with sarilumab led to numerically greater improvements in PtGA score versus treatment with placebo.

FIG. 18 graphically depicts the LSM change from baseline at week 52 for Pain Visual Analog Scale (VAS) score for patients treated with sarilumab and placebo. These results show that treatment with sarilumab led to numerically greater improvements in Pain VAS score versus treatment with placebo (with a higher score representing greater pain intensity).

FIG. 19 graphically depicts the LSM change from baseline at week 52 for EQ-5D index utility score and EQ VAS score for patients treated with sarilumab and placebo. These results show that sarilumab treatment led to a statistically greater improvement from baseline in EQ-5D index utility scores with a LSM difference versus placebo of 0.13 (P=0.0336). These results also show that sarilumab treatment led to a numerically greater improvement from baseline in EQ VAS score (with a higher score indicating better health).

FIG. 20 graphically depicts the proportion of patients without any PMR signs and symptoms per visit with sarilumab (200 mg Q2W+14-week GC taper) treatment and placebo (52-week GC treatment). With sarilumab treatment, the proportion of patients without PMR signs and symptoms increased versus placebo at week 2 and continued to increase over time until week 52. At each visit after baseline, the proportion of patients without any PMR signs and symptoms was higher in the sarilumab treatment group versus the placebo treatment group.

FIG. 21 graphically depicts the proportion of patients without any PMR signs and symptoms per visit with sarilumab (200 mg Q2W+14-week GC taper) treatment and placebo (52-week GC treatment) with the exclusion of patients who received rescue therapy. With sarilumab treatment, the proportion of patients without PMR signs and symptoms increased versus placebo at week 2 and continued to increase over time until week 52. At each visit after baseline, the proportion of patients without any PMR signs and symptoms was higher in the sarilumab treatment group. Additionally, a greater difference between the two treatment groups was observed in FIG. 21 than in FIG. 20, because patients receiving rescue therapy were excluded from the analysis in FIG. 21.

FIG. 22 graphically depicts the cumulative proportion of patients who received rescue therapy with sarilumab (200 mg Q2W+14-week GC taper) treatment and placebo (52-week GC treatment). These results show that the cumulative proportion of patients requiring rescue therapy was higher in the placebo treatment group at each timepoint after baseline up to week 52.

FIG. 23 graphically depicts the percent of patients treated with sarilumab and placebo that achieved sustained remission at week 52, assessed from week 12 to 52, week 16 to 52, and week 24 to 52.

FIGS. 24A-24B graphically depict the percent of patients treated with sarilumab and placebo that achieved disease remission (FIG. 24A) or absence of flare (FIG. 24B) at week 52, assessed from week 12 to 52, week 16 to 52, and week 24 to 52.

FIGS. 25A-25B graphically depict the percent of patients treated with sarilumab and placebo that achieved sustained normalization of CRP (FIG. 25A) or maintenance of steroid taper (FIG. 25B) at week 52, assessed from week 12 to 52, week 16 to 52, and week 24 to 52.

FIG. 26. graphically depicts the percent of patients treated with sarilumab or placebo that achieved GC-free resolution of PMR signs and symptoms by visit (not on rescue therapy by visit).

FIG. 27 graphically depicts the percent of patients treated with sarilumab or placebo that achieved no PMR signs and symptoms by visit.

FIG. 28 graphically depicts time to first flare after achieving clinical remission compared to the comparator arm.

FIG. 29 graphically depicts time to non-persistence (discontinuation of IL-6 inhibitors or conventional immunomodulatory medications, or switch) with IL-6 inhibitors (IL-6Ri) versus conventional immunomodulatory medications (cIM). These results show that time to discontinuation was significantly greater for IL-6Ri versus cIM patients (p=0.029).

FIG. 30 graphically depicts time and persistence on index PMR therapy (which is either IL-6Ri (sarilumab or tocilizumab) or CIM therapy, which shows a survival curve for persistence for second-line cohort post-propensity scoring match. CIM, conventional immunomodulators; IL-6Ri, interleukin-6 receptor inhibitor; PS, propensity scoring; 2 L, second-line.

FIG. 31 graphically depicts time and non-switch therapy, which shows survival curve for switching treatment for second-line cohort post PS match. CIM, conventional immunomodulators; IL-6Ri, interleukin-6 receptor inhibitor; PS, propensity scoring; 2 L, second-line.

### DETAILED DESCRIPTION

Before the disclosure is described, it is to be understood that the disclosure is not limited to particular methods and experimental conditions described, as such methods and conditions may vary. It is also to be understood that the terminology used herein is for the purpose of describing particular embodiments only, and is not intended to be limiting, because the scope of the disclosure will be limited only by the appended claims.

Unless defined otherwise, all technical and scientific terms used herein have the same meaning as commonly understood by one of ordinary skill in the art to which this disclosure belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc).

As used herein, the terms “treat,” “treating,” or the like, mean to alleviate symptoms, eliminate the causation of symptoms either on a temporary or permanent basis, or to prevent or slow the appearance of symptoms of the named disorder or condition.

As used herein, the term “PMR flare” refers to an increase in PMR symptoms. The symptoms of PMR can be selected from the group consisting of shoulder pain associated with inflammatory stiffness and hip girdle pain associated with inflammatory stiffness.

Although any methods and materials similar or equivalent to those described herein can be used in the practice of the disclosures herein, the typical methods and materials are now described. All publications mentioned herein are incorporated herein by reference in their entirety.

The present disclosure provides methods and compositions for treating polymyalgia rheumatica (PMR).

Polymyalgia rheumatica is a chronic, inflammatory disorder almost exclusively occurring in people over 50 years old. (Guggino et al. Pathogenesis of Polymyalgia Rheumatica. Reumatismo 2018 70(1):10-17) and Chatzigeorgiou C, et al. Comorbidity in polymyalgia rheumatica. Reumatismo. 2018 Mar. 27; 70(1):35-43., incorporated by reference herein in their entireties). Polymyalgia rheumatica presents with pain and stiffness of the shoulders and possibly the hip, elevated inflammatory makers (although occasionally normal), and a characteristic dramatic response to corticosteroids. Classification criteria for the diagnosis of polymyalgia rheumatica are described in the European League Against Rheumatism and the American College of Rheumatology (Dasgupta B, et al. 2012 provisional classification criteria for polymyalgia rheumatica: a European League Against Rheumatism/American College of Rheumatology collaborative initiative. Annals of the Rheumatic Diseases 2012; 71:484-492, incorporated by reference herein in its entirety). Criteria for classification include a patient who is 50 years old or older presenting with bilateral shoulder pain (not better explained by an alternative diagnosis) and elevated C-reactive protein (CRP) levels and/or elevated erythrocyte sedimentation rate (ESR). Additional criteria include the presence of morning stiffness for more than 45 minutes, and the presentation of new symptoms involving the hip (e.g., pain, tenderness, and limited movement). Additional classification criteria can include the lack of peripheral synovitis, lack of positive rheumatoid arthritis (RA) serology (rheumatoid factor (RF), anti-citrullinated protein antibody (ACPA), or both), and absence of peripheral joint pain. Additional classification criteria can include musculoskeletal ultrasound findings of bilateral shoulder abnormalities (subacromial bursitis/bicipital tenosynovitis/glenohumeral effusion) or abnormalities in one shoulder and hip (hip effusion, trochanteric bursitis).

Classification criteria for the diagnosis of polymyalgia rheumatica are described herein. See also the European League Against Rheumatism and the American College of Rheumatology, Dasgupta B, et al. 2012 provisional classification criteria for polymyalgia rheumatica: a European League Against Rheumatism/American College of Rheumatology collaborative initiative. Annals of the Rheumatic Diseases 2012; 71:484-492, incorporated herein in their entireties). Criteria for classification may include a patient who is 50 years old or older presenting with bilateral shoulder pain (not better explained by an alternative diagnosis) and elevated C-reactive protein (CRP) levels and/or elevated erythrocyte sedimentation rate (ESR). Additional criteria may include the presence of morning stiffness for more than 45 minutes, and the presentation of new symptoms involving the hip (e.g., pain, tenderness, and limited movement). (American College of Rheumatology criteria for rheumatic diseases including polymyalgia rheumatica can be found at www.rheumatology. org/Practice-Quality/Clinical-Support/Criteria/ACR-Endorsed-Criteria, incorporated herein in its entirety).

IL-6 interacts directly with the IL-6Rα subunit and the IL-6/IL-6Rα pair forms a high affinity complex with the glycoprotein 130 (gp130) subunit and initiates intracellular signaling via the Janus kinase (JAK)-signal transducer and activator of transcription (STAT) (JAK/STAT) and Ras/Raf/mitogen-activated protein kinase (MAPK) pathways. IL-6Rα also exists in a soluble form, which is involved in trans-signaling and allows IL-6 to affect cells that do not express IL-6Rα including synovial cells in the joint.

Sarilumab (SAR153191), also designated as REGN88, is a recombinant IgG1 kappa monoclonal antibody of fully human sequence directed against the alpha subunit of the IL-6 receptor complex (IL-6Ra). Sarilumab blocks the binding of IL-6 and interrupts the cytokine-mediated signaling cascade. Sarilumab is also known by the tradename KEVZARA®.

Tocilizumab (TCZ) is a humanized anti-interleukin-6 (IL-6) receptor monoclonal antibody that binds to the membrane-bound and soluble IL-6 receptors, inhibiting IL-6 signaling. Tocilizumab is also known by the tradename ACTEIVIRA®.

### Methods for Improving PMR-Associated Patient-Reported Outcome (PRO) Measures and Clinician-Reported Outcome (ClinRO) Measures

Methods for improving one or more PMR-associated patient-reported outcome (PRO) measures in a subject in need thereof, wherein the methods comprise administering a pharmaceutical composition comprising an IL-6R antagonist to the subject, are provided. Methods for improving one or more PMR-associated clinician-reported outcome (ClinRO) measures in a subject in need thereof, wherein the methods comprise administering a pharmaceutical composition comprising an IL-6R antagonist to the subject, are provided.

Examples of PMR-associated PRO measures include: (1) functional assessment of chronic illness therapy fatigue scale (FACIT-Fatigue), (2) EuroQol five-dimensional three-level questionnaire (EQ-5D-3L), (3) Short form-36v2 (SF-36v2), (4) health assessment questionnaire disability index (HAQ-DI), (5) Patient Global Assessment of disease activity (PtGA), and (6) Pain Visual Analog Scale (Pain-VAS).

An “improvement in a PMR-associated PRO measure” means an increase from baseline of one or more of FACIT-Fatigue score, EQ-5D-3L score, or SF-36v2 score; and/or a decrease from baseline of one or more of HAQ-DI score, PtGA score, or Pain-VAS score. As used herein, the term “baseline,” with regard to a PMR-associated PRO measure, means the numerical value of the PRO measure for a patient prior to or at the time of administration of a pharmaceutical composition comprising an IL-6R antagonist.

An example of a PMR-associated ClinRO measure includes physician global assessment of disease activity-visual analog scale (MD-VAS).

An “improvement in a PMR-associated ClinRO measure” means a decrease from baseline of MD-VAS score. As used herein, the term “baseline,” with regard to a PMR-associated ClinRO measure, means the numerical value of the ClinRO measure for a patient prior to or at the time of administration of a pharmaceutical composition comprising an IL-6R antagonist.

To determine whether a PMR-associated parameter has “improved,” the parameter is quantified at baseline and at a time point after administration of the pharmaceutical composition described herein. For example, a PMR-associated parameter may be measured at day 1, day 2, day 3, day 4, day 5, day 6, day 7, day 8, day 9, day 10, day 11, day 12, day 14, or at week 3, week 4, week 5, week 6, week 7, week 8, week 9, week 10, week 11, week 12, week 13, week 14, week 15, week 16, week 17, week 18, week 19, week 20, week 21, week 22, week 23, week 24, week 32, week 40, week 52, or longer, after the initial treatment with the pharmaceutical composition. The difference between the value of the parameter at a particular time point following initiation of treatment and the value of the parameter at baseline is used to establish whether there has been an “improvement” in the PMR-associated parameter (e.g., an increase or decrease, as the case may be, depending on the specific parameter being measured).

The terms “acquire” or “acquiring” as used herein, refer to obtaining possession of a physical entity, or a value, e.g., a numerical value, by “directly acquiring” or “indirectly acquiring” the physical entity or value, such as a PMR-associated parameter. “Directly acquiring” means performing a process (e.g., performing a synthetic or analytical method) to obtain the physical entity or value. “Indirectly acquiring” refers to receiving the physical entity or value from another party or source (e.g., a third-party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”).

Information that is acquired indirectly can be provided in the form of a report, e.g., supplied in paper or electronic form, such as from an online database or application (an “App”). The report or information can be provided by, for example, a healthcare institution, such as a hospital or clinic; or a healthcare provider, such as a doctor or nurse.

### The Functional Assessment of Chronic Illness Therapy Fatigue Scale

According to certain embodiments, administration of an IL-6R antagonist to a patient results in an increase from baseline of the functional assessment of chronic illness therapy fatigue scale (FACIT-Fatigue) score. FACIT-Fatigue is a generic PRO instrument which includes 13 items to measure fatigue. Each item is rated by patients on a 0 to 4 scale (0=not at all, 1=a little bit, 2=somewhat, 3=quite a bit, 4=very much). Scores are summarized to give a total score between and 52. The recall period is the last 7 days.

Therapeutic methods are provided that result in an increase in FACIT-Fatigue score from baseline. For example, administration of an IL-6R antagonist to a subject in need thereof causes an increase in FACIT-Fatigue score from baseline of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 41, 42, 43, 44, 45, 46, 47, 48, 49, or 50.

### EuroQol-5 Dimensions, 3 Level Version (EQ-5D-3L)

According to certain embodiments, administration of an IL-6R antagonist to a patient results in an increase from baseline of EQ-5D-3L. The EQ-5D-3L is a generic PRO instrument which measures health status (EuroQol Group, EuroQol-a new facility for the measurement of health-related quality of life, Health Policy 1990; 16(3):199-208). There are two components to the EQ-5D; a health utility index score derived from 5 items addressing mobility, self-care, usual activities, pain/discomfort, and anxiety/depression “today”, and a current (“right now”) general health status score derived from a single 0-100 Visual Analog Scale (VAS). EQ-5D index utility scores are anchored at 0 for death and 1 for perfect health. The VAS is anchored with ‘best imaginable health state’ and ‘worst imaginable health state.’

Therapeutic methods are provided that result in an increase in EQ VAS score from baseline. For example, administration of an IL-6R antagonist to a subject in need thereof causes an increase in EQ VAS score from baseline of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 points.

Therapeutic methods are provided that result in an increase in EQ-5D index utility score from baseline. For example, administration of an IL-6R antagonist to a subject in need thereof causes an increase in EQ-5D index utility score from baseline of about 0.05, 0.1, 0.15, 0.2. 0.25, 0.35, 0.4, 0.45, 0.5, 0.55, 0.6, 0.65, 0.7, 0.75, 0.8, 0.85, 0.9, or 0.95 points.

### Short Form 36v2 (SF-36v2)

According to certain embodiments, administration of an IL-6R antagonist to a patient results in an increase from baseline of Short form 36v2 (SF-36v2). The Short Form 36v2 (SF-36v2) is a short-form generic, 36-item PRO instrument that evaluates 8 multi-item dimensions of health: physical functioning (PF; 10 items), social functioning (SF; 2 items), role limitations due to physical problems (RP; 4 items), role limitations due to emotional problems (RE; 3 items), mental health (MH; 5 items), energy/vitality (VT; 4 items), bodily pain (BP; 2 items), and general health perception (GH; 5 items) (Ware, et al. The MOS 36-Item Short-Form Health Survey (SF-36): I. Conceptual Framework and Item Selection, Medical Care 1992; 30(6):473-483). For each dimension, item scores are coded, summed, and transformed on to a scale from 0 (worst possible health state measured by the questionnaire) to 100 (best possible health state). Two standardized summary scores can also be calculated from the SF-36v2; the physical component summary (PCS) and the mental health component summary (MCS) on a scale from 0-100 (See Maruish ME (2011) User's manual for the SF-36v2 Health Survey (3rd ed). Lincoln, RI: QualityMetric Incorporated).

Therapeutic methods are provided that result in an increase in SF-36v2 score from baseline. For example, administration of an IL-6R antagonist to a subject in need thereof causes an increase in SF-36v2 score from baseline of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 points.

### Health Assessment Questionnaire Disability Index (HAQ-DI)

The HAQ-DI was developed to assess physical functional status in adults with arthritis but is now commonly used among many rheumatologic conditions (See Wolfe F “A brief clinical health assessment instrument: CLINHAQ” Arthritis Rheum. 1989; 32 (suppl): S9 and Wolfe F. “Data collection and utilization: a methodology for clinical practice and clinical research” Rheumatoid arthritis: pathogenesis, assessment, outcome and treatment, New York: Marcel Dekker, 1994: 463-514). It contains 25 items: 20 4-point Likert-scale questions assessing 8 physical dimensions of activities of daily living (dressing and grooming, arising, eating, walking, hygiene, reaching, gripping, and errands and chores), 13 additional questions assessing use of assistive devices, and 8 additional questions assessing help received from another. The recall period is the last week. To calculate the HAQ-DI Score, there are 3 steps: sum the 8 category scores by using the highest sub-category score from each category; adjust for use of aids/devices and/or help from another person when indicated; and divide the summed category scores by the number of categories answered (must be a minimum of 6) to obtain a HAQ-DI score of 0-3 (3=worst functioning). In addition, the HAQ-DI has two additional questions, measured on 0-100 scales: How much pain have you had IN THE PAST WEEK? Please rate how well you are doing on a scale of 0 to 100 (0 represents “very well” and 100 represents “very poor” health). These questions, measuring pain and global assessment respectively, are independently scored.

Therapeutic methods are provided that result in a decrease in HAQ-DI score from baseline. For example, administration of an IL-6R antagonist to a subject in need thereof causes a decrease in HAQ-DI score from baseline of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 96, 97, 98, 99, or 100 points.

### Patient Global Assessment of Disease Activity (PtGA)

PtGA is a single question with a score of 0 to 100 that focuses on overall health or disease activity from the patient perspective. A higher score represents a higher level of disease activity or a worse global health.

Therapeutic methods are provided that result in a decrease in PtGA score from baseline. For example, administration of an IL-6R antagonist to a subject in need thereof causes a decrease in PtGA score from baseline of about 1, 2, 3, 4, 5, 6, 7, 8, 9, 10, 11, 12, 13, 14, 15, 16, 17, 18, 19, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 points.

### Pain Visual Analog Scale (VAS)

Pain VAS is a unidimensional, patient reported measure of pain intensity (See Delgado et al. “Validation of digital visual analog scale pain scoring with a traditional paper-based visual analog scale in adults” Journal of the American Academy of Orthopaedic Surgeons, March; 2(3)). Pain VAS score ranges from 0 to 100 and a higher score indicates greater pain intensity.

Therapeutic methods are provided that result in a decrease in Pain VAS score from baseline. For example, administration of an IL-6R antagonist to a subject in need thereof causes a decrease in Pain VAS score from baseline of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 points.

### Physician Global Assessment of Disease Activity-Visual Analog Scale [MD-VAS]

In the MD-VAS, a physician rates the patient's disease activity on an anchored 100 mm horizontal VAS where 0 is considered not active and 100 is considered the most active (See Huskisson et al. “Vertical or Horizontal Visual Analogue Scales” Ann Rheum Dis. 1979 December; 38(6):560).

Therapeutic methods are provided that result in a decrease in MD-VAS score from baseline. For example, administration of an IL-6R antagonist to a subject in need thereof causes a decrease in HAQ-DI score from baseline of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 points.

The methods described herein may further improve one or more other PMR-associated outcomes, including, without limitation, PMR activity score (PMR-AS), glucocorticoid toxicity index (GTI), cumulative corticosteroid dose and time to PMR flare.

### PMR Activity Score (PMR-AS)

PMR-AS is calculated as the sum of CRP (mg/dL), visual analog score (VAS) for pain (0 to 10), VAS for physician's assessment (0 to 10), duration of morning stiffness (MST [min] X 0.1), and the ability to elevate the upper limbs (EUL [3-0]).

Therapeutic methods are provided that result in a decrease in PMR-AS score from baseline. For example, administration of an IL-6R antagonist to a subject in need thereof causes a decrease in PMR-AS score from baseline of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 points.

### Glucocorticoid Toxicity Index

Glucocorticoid Toxicity Index (GTI) is a composite scale designed to assess glucocorticoid-related morbidity. GTI-cumulative worsening score (CWS) captures cumulative glucocorticoid toxicity regardless of whether it is permanent or transient. The GTI-CWS can only increase or remain the same over time. A lower score indicates lower glucocorticoid toxicity. The GTI-aggregate improvement score (AIS) captures both worsening and improvement in glucocorticoid toxicity. New or worsening toxicities contribute a positive score and improvements in existing toxicities contribute a negative score. A lower score indicates lower glucocorticoid toxicity.

Therapeutic methods are provided that result in a decrease in GTI-CWS or GTI-AIS score from baseline. For example, administration of an IL-6R antagonist to a subject in need thereof causes a decrease in GTI-CWS or GTI-AIS score from baseline of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 points.

### Cumulative Corticosteroid Dose

The cumulative corticosteroid dose is a measure of a patient's exposure to corticosteroids over a period of time, such as about 1, 2, 3, 4, 5, 6, 7, 8, 9, 10, 11, 12, 13, 14, 15, 16, 17, 18, 19, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 weeks.

Therapeutic methods are provided that result in a decrease cumulative corticosteroid dose over a period of time with treatment with the IL-6R antagonist compared to treatment without the IL-6R antagonist. For example, administration of an IL-6R antagonist to a subject in need thereof causes a decrease in cumulative corticosteroid dose of about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, or 3000 mg.

### Time to PMR Flare

Therapeutic methods are provided that result in an increase in the amount of time until a patient experiences a PMR flare. For example, administration of an IL-6R antagonist to a subject in need thereof causes an increase in the amount of time until a patient experiences a PMR flare of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 81, 82, 83, 84, 85, 86, 87, 88, 89, 90, 91, 92, 93, 94, 95, 96, 97, 98, 99, or 100 weeks.

### Methods of Administration and Formulations

The methods described herein comprise administering a therapeutically effective amount of an anti-IL-6R antibody to a subject. As used herein, the phrase “therapeutically effective amount” means a dose of the therapeutic that results in treatment of polymyalgia rheumatica. As used herein, “treating” refers to causing a detectable improvement in one or more symptoms associated with polymyalgia rheumatica or causing a biological effect (e.g., a decrease in the level of a particular biomarker) that is correlated with the underlying pathologic mechanism(s) giving rise to the condition or symptom(s). For example, the following symptoms or conditions are associated with polymyalgia rheumatica: bilateral shoulder pain, pain or tenderness in the hip and limited hip movement, elevated C-reactive protein (CRP) levels, elevated erythrocyte sedimentation rate (ESR), and the presence of morning stiffness for more than a number of minutes (e.g., 30 or 45 minutes).

An “improvement” in an PMR-associated symptom in various embodiments refers reduction in the incidence of the PMR symptom which may correlate with an improvement in one or more P-associated test, score or metric (as described here). For example, the improvement may correlate an increase from baseline of one or more of C-reactive protein (CRP), ESR, IL-6, soluble IL-6R, and/or markers of inflammation and disease activity over time as assessed in circulating immune cell types, circulating proteins, and gene expression changes. As used herein, the term “baseline,” with regard to a PMR-associated parameter, means the numerical value of the PMR-associated parameter for a patient prior to or at the time of administration of the antibody described herein.

A detectable “improvement” can also be detected using at least one test, score or metric described herein. In various embodiments, the improvement is detected by a reduction in a symptom of PMR selected from the group consisting of: morning stiffness, pain in the neck, pain in the shoulder, pain in the hip girdles, limited range of motion of the shoulders, limited range of motion in the hip girdles, constitutional symptoms (e.g., fatigue, weight loss and low grade fever), and other features judged by the clinician-investigator to be consistent with a PMR flare. In various embodiments, the improvement is detected by using at least one selected from the group consisting of: a patient-reported outcome (PRO) questionnaire, a functional assessment of chronic illness therapy fatigue scale (FACIT-Fatigue), EQ-5D-3L, Short Form 36v2, HAQ-DI, PMR-AS, and physician global assessment of disease activity (e.g., Visual Analog Scale [MD-VAS]).

In various embodiments, a detectable improvement is defined as a sustained remission of the disease. As used herein, a “sustained remission” of PMR in a subject is defined as being one or several of the following: (i) disease remission, in particular by week 12 after initiation of the treatment with a therapeutically effective amount of an anti-IL-6R antibody (i.e., absence of signs and symptoms of PMR in the subject); (ii) absence of disease flare; (iii)C-reactive protein normalization, in particular from weeks 12 to 52; or (iv) adherence to a steroid taper protocol, in particular a glucocorticoid taper (e.g., prednisone taper) protocol, in particular from weeks 12 to 52.

In another example, a treatment has not been effective when a dose of anti-IL-6R antibody does not result in a detectable improvement in one or more parameters or symptoms associated with PMR or which does not cause a biological effect that is correlated with the underlying pathologic mechanism(s) giving rise to the condition or symptom(s) of PMR.

In various embodiments, an IL-6R antibody is administered subcutaneously. In various embodiments, the IL-6R antibody is sarilumab.

In various embodiments, a therapeutically effective amount of anti-IL-6R antibody that is administered to the subject will vary depending upon the age and the size (e.g., body weight or body surface area) of the subject as well as the route of administration and other factors well known to those of ordinary skill in the art.

In various embodiments, the dose is a fixed dose regardless of the body weight or surface area of the subject. In various embodiments, the subject is at least 50 years old. In various embodiments, the subject is older than 50 years old.

The disclosure provides methods of using therapeutic compositions comprising anti-IL-6R antibodies or antigen-binding fragments thereof and, optionally, one or more additional therapeutic agents. The therapeutic compositions of the present disclosure will be administered with suitable carriers, excipients, and/or other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN®), DNA conjugates, anhydrous absorption pastes, oil-in-water and water-in-oil emulsions, emulsions carbowax (polyethylene glycols of various molecular weights), semi-solid gels, and semi-solid mixtures containing carbowax.

Various delivery systems are known and can be used to administer pharmaceutical compositions provided herein, e.g., encapsulation in liposomes, microparticles, microcapsules, receptor mediated endocytosis. Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc). and may be administered together with other biologically active agents. Administration can be systemic or local. The IL-6R antibody can be administered subcutaneously.

The pharmaceutical composition can also be delivered in a vesicle, such as a liposome. In certain embodiments, the pharmaceutical composition can be delivered in a controlled release system, for example, with the use of a pump or polymeric materials. In certain embodiments, a controlled release system can be placed in proximity of the composition's target, thus requiring only a fraction of the systemic dose.

The injectable preparations may include dosage forms for intravenous, subcutaneous, intracutaneous and intramuscular injections, local injection, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc). As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

The antibody is typically formulated as described herein and in international publication number WO2011/085158, incorporated herein by reference in its entirety.

In various embodiments, the antibody is administered as an aqueous buffered solution at about pH 6.0 containing
about 21 mM histidine,
about 45 mM arginine,
about 0.2% (w/v) polysorbate 20,
about 5% (w/v) sucrose, and
between about 100 mg/mL and about 200 mg/mL of the antibody.

In another embodiment, the antibody is administered as an aqueous buffered solution at about pH 6.0 containing
about 21 mM histidine,
about 45 mM arginine,
about 0.2% (w/v) polysorbate 20,
about 5% (w/v) sucrose, and
at least about 130 mg/mL of the antibody.

In another embodiment, the antibody is administered as an aqueous buffered solution at about pH 6.0 containing
about 21 mM histidine,
about 45 mM arginine,
about 0.2% (w/v) polysorbate 20,
about 5% (w/v) sucrose, and
about 131.6 mg/mL of the antibody.

In another embodiment, the antibody is administered as an aqueous buffered solution at about pH 6.0 containing
about 21 mM histidine,
about 45 mM arginine,
about 0.2% (w/v) polysorbate 20,
about 5% (w/v) sucrose; and
about 175 mg/mL of the antibody.

In other embodiments, the antibody is administered as an aqueous buffered solution at pH 6.0 containing
21 mM histidine,
45 mM arginine,
0.2% (w/v) polysorbate 20,
5% (w/v) sucrose, and
between 100 mg/mL and 200 mg/mL of the antibody.

In another embodiment, the antibody is administered as an aqueous buffered solution at pH 6.0 containing
21 mM histidine,
45 mM arginine,
0.2% (w/v) polysorbate 20,
5% (w/v) sucrose, and
at least 130 mg/mL of the antibody.

In another embodiment, the antibody is administered as an aqueous buffered solution at pH 6.0 containing
21 mM histidine,
45 mM arginine,
0.2% (w/v) polysorbate 20,
5% (w/v) sucrose, and
131.6 mg/mL of the antibody.

In another embodiment, the antibody is administered as an aqueous buffered solution at pH 6.0 containing
21 mM histidine,
45 mM arginine,
0.2% (w/v) polysorbate 20,
5% (w/v) sucrose; and
175 mg/mL of the antibody.

In various embodiments, the antibody is administered in a stable pharmaceutical formulation comprising: (i) histidine at a concentration of from 25 mM to 100 mM; (ii) arginine at a concentration of from 25 mM to 50 mM; (iii) sucrose in an amount of from 3% to 10% w/v; and (iv) polysorbate 20 in an amount of from 0.1% to 0.2%, wherein the formulation has a pH of about 5.8, about 6.0, or about 6.2, and at least 90% of the native form of the antibody is recovered after 1 month of storage at 45° C., as determined by size exclusion chromatography. In various embodiments, about 200 mg of the antibody (e.g., sarilumab) is administered to the subject. In various embodiments, about 150 mg of the antibody (e.g., sarilumab) is administered to the subject.

In various embodiments, the antibody is administered in a stable pharmaceutical formulation comprising: (i) histidine at a concentration of from about 10 mM to about 25 mM; (ii) arginine at a concentration of from about 25 mM to about 50 mM; (iii) sucrose in an amount of from about 5% to about 10% w/v; and (iv) polysorbate in an amount of from about 0.1% to about 0.2% w/v, wherein the formulation has a pH of about 5.8, about 6.0, or about 6.2, and at least 90% of the native form of the antibody is recovered after 1 month of storage at 45° C., as determined by size exclusion chromatography. In various embodiments, about 200 mg of the antibody (e.g., sarilumab) is administered to the subject. In various embodiments, about 150 mg of the antibody (e.g., sarilumab) is administered to the subject.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

In various embodiments, the anti-IL-6R antibody (or pharmaceutical formulation comprising the antibody) can be administered to the patient using any acceptable device or mechanism. For example, the administration can be accomplished using a syringe and needle or with a reusable pen and/or autoinjector delivery device. The methods of the present disclosure include the use of numerous reusable pen and/or autoinjector delivery devices to administer an anti-IL-6R antibody (or pharmaceutical formulation comprising the antibody). Examples of such devices include, but are not limited to AUTOPEN® (Owen Mumford, Inc., Woodstock, UK), DISETRONIC® pen (Disetronic Medical Systems, Bergdorf, Switzerland), HUMALOG MIX® pen, HUMALOG® pen, HUMALIN® 70/30 pen (Eli Lilly and Co., Indianapolis, IN), NOVOPEN® I, II and III (Novo Nordisk, Copenhagen, Denmark), NOVOPEN JUNIOR® (Novo Nordisk, Copenhagen, Denmark), BD® pen (Becton Dickinson, Franklin Lakes, NJ), OPTIPEN®, OPTIPEN PRO®, OPTIPEN STARLET®, and OPTICLIK® (Sanofi-Aventis, Frankfurt, Germany). Examples of disposable pen and/or autoinjector delivery devices having applications in subcutaneous delivery of a pharmaceutical composition of the present disclosure include, but are not limited to the SOLOSTAR® pen (Sanofi-Aventis), the FLEXPEN® (Novo Nordisk), and the KWIKPEN® (Eli Lilly), the SURECLICK® Autoinjector (Amgen, Thousand Oaks, CA), the PENLET® (Haselmeier, Stuttgart, Germany), the EPIPEN® (Dey, L. P.)., and the HUMIRA® Pen (AbbVie Inc., North Chicago, IL), to name only a few.

In various embodiments, the antibody is administered with a prefilled syringe. In various embodiments, the antibody is administered with a prefilled syringe containing a safety system. For example, the safety system prevents an accidental needle-stick injury. In various embodiments, the antibody is administered with a prefilled syringe containing an EMS safety system (West Pharmaceutical Services Inc).

In various embodiments, the antibody is administered with an auto-injector. In various embodiments, the antibody is administered with an auto-injector featuring the PUSHCLICK® technology (SHL Group). In various embodiments, the auto-injector is a device comprising a syringe that allows for administration of a dose of the composition and/or antibody to a subject.

The use of a microinfusor to deliver an anti-IL-6R antibody (or pharmaceutical formulation comprising the antibody) to a patient is also contemplated herein. As used herein, the term “microinfusor” means a subcutaneous delivery device designed to slowly administer large volumes (e.g., up to about 2.5 mL or more) of a therapeutic formulation over a prolonged period of time (e.g., about 10, 15, 20, 25, 30 or more minutes). Microinfusors are particularly useful for the delivery of large doses of therapeutic proteins contained within high concentration (e.g., about 100, 125, 150, 175, 200 mg/mL or more) and/or viscous solutions.

In various embodiments, an inadequate response to prior treatment refers to subjects whose pain is not well controlled after receiving the prior treatment at the maximum tolerated typical dose. In an embodiment, an inadequate response to prior treatment refers to subjects who have moderate or high disease activity and features of poor prognosis despite prior treatment. In various embodiments, an inadequate response to prior treatment refers to subjects with a pain symptom (e.g., any symptom listed herein) that has not improved or that has worsened despite prior treatment.

### Dosage

The amount of IL-6R antagonist (e.g., anti-IL-6R antibody) administered to a subject according to the methods described herein is, generally, a therapeutically effective amount. As used herein, the phrase “therapeutically effective amount” means an amount of IL-6R antagonist that results in improvement in one or more PMR-associated PRO measures or ClinRO measures (as defined elsewhere herein). A “therapeutically effective amount” also includes an amount of IL-6R antagonist that inhibits, prevents, lessens, or delays the progression of PMR in a subject. In some embodiments, a therapeutically effective amount of anti-IL-6R antibody reduces the dose of corticosteroid (e.g., prednisone) administered to a subject. In the case of an anti-IL-6R antibody, a therapeutically effective amount can be from about 0.05 mg to about 700 mg, e.g., about 0.05 mg, about 0.1 mg, about 1.0 mg, about 1.5 mg, about 2.0 mg, about 3.0 mg, about 5.0 mg, about 7.0 mg, about 10 mg, about 20 mg, about 30 mg, about 40 mg, about 50 mg, about 60 mg, about mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, or about 700 mg of the anti-IL-6R antibody. In certain embodiments, 200 mg of an anti-IL-6R antibody is administered. In certain embodiments, 150 mg of an anti-IL-6R antibody is administered. In certain embodiments, 300 mg of an anti-IL-6R antibody is administered.

The amount of IL-6R antagonist contained within the individual doses may be expressed in terms of milligrams of antibody per kilogram of subject body weight (i.e., mg/kg). For example, the IL-6R antagonist may be administered to a patient at a dose of about 0.0001 to about 10 mg/kg of subject body weight. For example, the IL-6R antagonist can be administered at a dose of 1 mg/kg, 2 mg/kg, 3 mg/kg, 4 mg/kg, 5 mg/kg or 6 mg/kg.

In certain embodiments, the initial dose is about the same as the loading dose. In certain embodiments, the initial dose is about 1.1×, about 1.2×, about 1.3×, about 1.4×, about 1.5×, about 1.6×, about 1.7×, about 1.8×, about 1.9×, about 2.0×, about 2.5×, about 3.0×, or more of the loading dose.

In certain embodiments, two or more (e.g., 2, 3, 4, or 5 or more) doses are administered at the beginning of the treatment regimen as “initial doses” or “loading doses” followed by subsequent doses that are administered on a less frequent basis (e.g., “maintenance doses”). In one embodiment, the maintenance dose may be lower than the loading or initial dose.

In certain exemplary embodiments, the IL-6R antagonist is administered at a dose of about 150 mg or about 200 mg. In particularly exemplary embodiments, the IL-6R antagonist is administered at an initial dose of about 200 mg and one or more secondary doses or maintenance doses of about 200 mg, and the secondary doses are administered every other week (q2w).

In certain exemplary embodiments, a subject is an adult, and the IL-6R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In exemplary embodiments, a subject is an adult, and the IL-6R antagonist is administered at an initial dose of about 600 mg and one or more secondary doses or maintenance doses of about 300 mg, and the secondary doses are administered every other week (q2w). In other exemplary embodiments, a subject is an adult, and the IL-6R antagonist is administered at an initial dose of about 400 mg and one or more secondary doses or maintenance doses of about 200 mg, and the secondary doses are administered every other week (q2w). In certain embodiments, a subject is an adult, the initial dose comprises about 300 mg of the IL-6R antagonist, and the one or more subsequent doses comprise about 300 mg of the IL-6R antagonist administered every other week.

In certain exemplary embodiments, an IL-6R antagonist is administered at a concentration of 150 mg/mL using a prefilled device. In some embodiments, a 150 mg/mL IL-6R antagonist solution in a pre-filled device is used to deliver about 300 mg IL-6R antagonist in a 2 mL injection. In certain exemplary embodiments, an IL-6R antagonist is administered at a concentration of 175 mg/mL using a prefilled device. In some embodiments, a 175 mg/mL IL-6R antagonist solution in a pre-filled device is used to deliver about 200 mg IL-6R antagonist in a 1.14 mL injection. In certain exemplary embodiments, an IL-6R antagonist is administered at a concentration of 131 mg/mL using a prefilled device. In some embodiments, a 131 mg/mL IL-6R antagonist solution in a pre-filled device is used to deliver about 150 mg IL-6R antagonist in a 1.14 mL injection.

### Combination Therapies

Certain embodiments of the methods described herein comprise administering to the subject one or more additional therapeutic agents in combination with the IL-6R antagonist. As used herein, the expression “in combination with” means that the additional therapeutic agents are administered before, after, or concurrent with the pharmaceutical composition comprising the IL-6R antagonist. In some embodiments, the term “in combination with” includes sequential or concomitant administration of an IL-6R antagonist and a second therapeutic agent. Methods to treat PMR or an associated condition or complication comprising administration of an IL-6R antagonist in combination with a second therapeutic agent for additive or synergistic activity, are provided.

For example, when administered “before” the pharmaceutical composition comprising the IL-6R antagonist, the additional therapeutic agent may be administered about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes, or about 10 minutes prior to the administration of the pharmaceutical composition comprising the IL-6R antagonist. When administered “after” the pharmaceutical composition comprising the IL-6R antagonist, the additional therapeutic agent may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours, or about 72 hours after the administration of the pharmaceutical composition comprising the IL-6R antagonist. Administration “concurrent” with the pharmaceutical composition comprising the IL-6R antagonist means that the additional therapeutic agent is administered to the subject in a separate dosage form within less than 5 minutes (before, after, or at the same time) of administration of the pharmaceutical composition comprising the IL-6R antagonist, or administered to the subject as a single combined dosage formulation comprising both the additional therapeutic agent and the IL-6R antagonist.

In exemplary embodiments, an additional therapeutic agent administered in combination with the IL-6R antagonist is a background therapy. In some embodiments, a background therapy includes a steroid. In exemplary embodiments, the background therapy is a corticosteroid. Corticosteroids are steroid hormones produced in the adrenal cortex of vertebrates, and synthetic analogues of these hormones. Corticosteroids include prednisone, hydrocortisone, hydrocortisone acetate, cortisone acetate, tixocortol pivalate, prednisolone and methylprednisolone. In some embodiments, the corticosteroid is prednisone. According to other embodiments, the corticosteroid can also be selected from triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-valerate, halometasone, alclometasone dipropionate, betamethasone valerate, betamethasone dipropionate, prednicarbate, clobetasone-17-buty rate, clobetasol-17-propionate, fluocortolone caproate, fluocortolone pivalate, fluprednidene acetate, hydrocortisone-17-butyrate, hydrocortisone-17-aceponate, hydrocortisone-17-buteprate, ciclesonide and prednicarbate.

In certain embodiments, the method leads to a reduced need of the background therapy. Reducing the dose of a background therapy may also be termed “tapering”. For example, in certain embodiments, the method leads to reduced dose and/or reduced frequency of the background therapy. In exemplary embodiments, the method leads to reduced dose and/or reduced frequency of corticosteroid background therapy.

In certain embodiments, the method leads to a discontinuation of the background therapy. In exemplary embodiments, the method leads to a discontinuation of corticosteroid background therapy.

In certain embodiments, the method is used to treat PMR (or one or more symptoms of PMR) in a subject that has had an inadequate response to a background therapy, in particular to steroids such as corticosteroids. In exemplary embodiments, the method leads to treatment of PMR (or one or more symptoms of PMR) with a reduced need or without the need for corticosteroid background therapy.

In certain embodiments, the method is used to treat PMR (or one or more symptoms of PMR) in a subject who cannot tolerate a background therapy taper, in particular a steroid taper such as a corticosteroid taper. In exemplary embodiments, the method leads to treatment of PMR (or one or more symptoms of PMR) with a reduced need or without the need for corticosteroid background therapy.

In certain embodiments, the method is used to treat PMR (or one or more symptoms of PMR) in a subject that has had an inadequate response to a background therapy, in particular to steroids such as corticosteroids and/or in a subject who cannot tolerate a background therapy taper, in particular a steroid taper such as a corticosteroid taper. In exemplary embodiments, the method leads to treatment of PMR (or one or more symptoms of PMR) with a reduced need or without the need for corticosteroid background therapy. In some embodiments, the method is used to treat PMR (or one or more symptoms of PMR) in an adult subject who has had an inadequate response to corticosteroids or who cannot tolerate corticosteroid taper.

In some embodiments, the corticosteroid background therapy can be administered from about 7.5 mg/day to about 80 mg/day. In certain embodiments, the corticosteroid background therapy can be administered from 15 mg/day to 20 mg/day, from about 20 mg/day to about mg/day, and from about 35 mg/day to about 80 mg/day. In some embodiments, the corticosteroid background therapy is administered at a dosage of about 7.5 mg/day, about mg/day, about 12.5 mg/day, about 15 mg/day, about 20 mg/day, about 25 mg/day, about mg/day, about 35 mg/day, about 40 mg/day, about 45 mg/day, about 50 mg/day, about mg/day, about 60 mg/day, about 65 mg/day, about 70 mg/day, about 75 mg/day, or about mg/day. In an exemplary embodiment, the corticosteroid background therapy is administered at a dosage of about 15 mg/day.

In some embodiments, the dose of the background therapy is tapered with treatment with the IL-6R antagonist. Polymyalgia rheumatica patients attempting to taper the daily dosage of corticosteroid treatment to lower dosages of corticosteroid can experience at least one episode of flare, e.g., reducing the dose such that the patient no longer experiences shoulder pain, hip girdle pain, or both, along with inflammatory stiffness lasting more than a certain period of time (e.g., 45 minutes) in the morning. Treatment with an IL-6R antibody or antibody fragment, as described herein, can lessen the episodes of flare as a subject's daily dosage of corticosteroid treatment is tapered, or decreased, over time.

The additional therapeutic agent may be, e.g., another IL-6R antagonist, an IL-6 antagonist, a steroid, etc. In an exemplary embodiment, the additional therapeutic is a corticosteroid. In a further exemplary embodiment, the additional therapeutic is prednisone.

In some embodiments, an additional therapeutic agent administered in combination with the IL-6R antagonist is a vaccine. In certain exemplary embodiments, the vaccine is a viral vaccine or a bacterial vaccine. In certain exemplary embodiments, the vaccine is a live (e.g., live-attenuated) viral vaccine or a live (e.g., live-attenuated) bacterial vaccine.

Suitable vaccines include, but are not limited to adenovirus, anthrax (e.g., AVA vaccine (BioThrax)), cholera (e.g., Vaxchora), diphtheria (e.g., DTaP (Daptacel, Infanrix), Td (Tenivac, generic), DT (generic), Tdap (Adacel, Boostrix), DTaP-IPV (Kinrix, Quadracel), DTaP-HepB-IPV (Pediarix), DTaP-IPV/Hib (Pentacel)), hepatitis A (e.g., HepA (Havrix, Vaqta), HepA-HepB (Twinrix)), hepatitis B (e.g., HepB (Engerix-B, Recombivax HB, Heplisav-B), DTaP-HepB-IPV (Pediarix), HepA-HepB (Twinrix)), Haemophilus influenzae type b (Hib) (e.g., Hib (ActHIB, PedvaxHlB, Hiberix), DTaP-IPV/Hib (Pentacel)), human papillomavirus (HPV) (e.g., HPV9 (Gardasil 9)), influenza (flu) (e.g., IIV (also called IIV3, IIV4, RIV3, RIV4 and ccIIV4) (Afluria, Fluad, Flublok, Flucelvax, FluLaval, Fluarix, Fluvirin, Fluzone, Fluzone High-Dose, Fluzone Intradermal), LAIV (FluMist)), Japanese encephalitis (e.g., JE (Ixiaro)), measles (e.g., MMR (M-M-R II), MMRV (ProQuad)), meningococcus (e.g., MenACWY (Menactra, Menveo), MenB (Bexsero, Trumenba)), mumps (e.g., MMR (M-M-R II), MMRV (ProQuad)), pertussis (e.g., DTaP (Daptacel, Infanrix), Tdap (Adacel, Boostrix), DTaP-IPV (Kinrix, Quadracel), DTaP-HepB-IPV (Pediarix), DTaP-IPV/Hib (Pentacel)), pneumococcus (e.g., PCV13 (Prevnar13), PPSV23 (Pneumovax 23)), polio (e.g., Polio (Ipol), DTaP-IPV (Kinrix, Quadracel), DTaP-HepB-IPV (Pediarix), DTaP-IPV/Hib (Pentacel)), rabies (e.g., Rabies (Imovax Rabies, RabAvert)), rotavirus (e.g., RV1 (Rotarix), RV5 (RotaTeq)), rubella (e.g., MMR (M-M-R II), MMRV (ProQuad)), shingles (e.g., ZVL (Zostavax), RZV (Shingrix)), smallpox (e.g., Vaccinia (ACAM2000)), tetanus (e.g., DTaP (Daptacel, Infanrix), Td (Tenivac, generic), DT (generic), Tdap (Adacel, Boostrix), DTaP-IPV (Kinrix, Quadracel), DTaP-HepB-IPV (Pediarix), DTaP-IPV/Hib (Pentacel)), tuberculosis, typhoid fever (e.g., Typhoid Oral (Vivotif), Typhoid Polysaccharide (Typhim Vi)), varicella (e.g., VAR (Varivax), MMRV (ProQuad)), yellow fever (e.g., YF (YF-Vax)) and the like. Suitable vaccines are also listed at the US Centers for Disease Control vaccine list, incorporated herein in its entirety for all purposes (cdc.gov/vaccines/vpd/vaccines-list.html). In some embodiments, the vaccine is for tetanus, diphtheria, pertussis and/or seasonal trivalent/quadrivalent influenza vaccine.

In some embodiments, the vaccine is an inactivated vaccine, a recombinant vaccine, a conjugate vaccine, a subunit vaccine, a polysaccharide vaccine, or a toxoid vaccine. In some embodiments, the vaccine is a yellow fever vaccine. In some embodiments, the subject treated with the vaccine is concurrently treated for PMR with an IL-6R antagonist.

In certain embodiments, treatment with an IL-6R antagonist is suspended or terminated prior to treatment with the vaccine. In certain embodiments, treatment with the IL-6R antagonist is suspended about 1 to about 9 (e.g., about 1, about 1½, about 2, about 2½, about 3, about 3½, about 4, about 4½, about 5, about 5½, about 6, about 6½, about 7, about 7½, about 8, about 8½, about 9, or more) weeks prior to administration of the vaccine. In some embodiments, treatment with the IL-6R antagonist is suspended about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, or about 60 days prior to administration of the vaccine.

In certain embodiments, treatment with the IL-6R antagonist is resumed subsequent to treatment with the vaccine. In certain embodiments, treatment with the IL-6R antagonist is resumed about 1 to about 14 (e.g., about 1, about 1½, about 2, about 2½, about 3, about 3½, about 4, about 4½, about 5, about 5½, about 6, about 6½, about 7, about 7½, about 8, about 8½, about 9, about 9½, about 10, about 10½, about 11, about 11½, about 12, about 12½, about 13, about 13½, about 14, about 14½, or more) weeks subsequent to administration of the vaccine. In some embodiments, treatment with the IL-6R antagonist is resumed about 1, about 2, about 3, about 4, about 5, about 6, about 7, about 8, about 9, about 10, about 11, about 12, about 13, about 14, about about 16, about 17, about 18, about 19, about 20, about 21, about 22, about 23, about 24, about about 26, about 27, about 28, about 29, about 30, about 31, about 32, about 33, about 34, about about 36, about 37, about 38, about 39, about 40, about 41, about 42, about 43, about 44, about about 46, about 47, about 48, about 49, about 50, about 51, about 52, about 53, about 54, about about 56, about 57, about 58, about 59, about 60, about 61, about 62, about 63, about 64, about about 66, about 67, about 68, about 69, about 70, about 71, about 72, about 73, about 74, about about 76, about 77, about 78, about 79, about 80, about 81, about 82, about 83, about 84, about about 86, about 87, about 88, about 89, or about 90 days subsequent to administration of the vaccine.

In certain embodiments, the effectiveness of the IL-6R antagonist is not decreased by administration in combination with the vaccine, or by subsequent administration of the vaccine.

In some embodiments, the effectiveness of the vaccine is not decreased by administration in combination with the IL-6R antagonist, or by previous and/or subsequent administration of the IL-6R antagonist. In some embodiments, the subject develops seroprotective neutralization titers to the vaccine when the vaccine is co-administered with the IL-6R antagonist.

In certain exemplary embodiments, a subject is administered a vaccine described herein, wherein before, during, or after administration of the vaccine, the subject is administered at least one dose of IL-6R antagonist.

### Administration Regimens

According to certain embodiments, multiple doses of an IL-6R antagonist may be administered to a subject over a defined time course. Such methods comprise sequentially administering to a subject multiple doses of an IL-6R antagonist. As used herein, “sequentially administering” means that each dose of IL-6R antagonist is administered to the subject at a different point in time, e.g., on different days separated by a predetermined interval (e.g., hours, days, weeks, or months). Methods that comprise sequentially administering to the patient a single initial dose of an IL-6R antagonist, followed by one or more secondary doses of the IL-6R antagonist, and optionally followed by one or more tertiary doses of the IL-6R antagonist, are provided.

Methods comprising administering to a subject a pharmaceutical composition comprising an IL-6R antagonist at a dosing frequency of about four times a week, twice a week, once a week (q1w), once every two weeks (every two weeks is used interchangeably with every other week, bi-weekly or q2w), once every three weeks (tri-weekly or q3w), once every four weeks (monthly or q4w), once every five weeks (q5w), once every six weeks (q6w), once every seven weeks (q7w), once every eight weeks (q8w), once every nine weeks (q9w), once every ten weeks (g10w), once every eleven weeks (q11w), once every twelve weeks (q12w), or less frequently so long as a therapeutic response is achieved, are provided.

In certain embodiments involving the administration of a pharmaceutical composition comprising an anti-IL-6R antibody, once a week dosing of an amount of about 150 mg or about 200 mg can be employed. In other embodiments involving the administration of a pharmaceutical composition comprising an anti-IL-6R antibody, once every two weeks dosing (every two weeks is used interchangeably with every other week, bi-weekly or q2w) of an amount of about 150 mg, or about 200 mg can be employed. In other embodiments involving the administration of a pharmaceutical composition comprising an anti-IL-6R antibody, once every three weeks dosing of an amount of about 150 mg or about 200 mg can be employed. In other embodiments involving the administration of a pharmaceutical composition comprising an anti-IL-6R antibody, once every four weeks dosing (monthly dosing) of an amount of about 150 mg or about 200 mg can be employed. In other embodiments involving the administration of a pharmaceutical composition comprising an anti-IL-6R antibody, once every five weeks dosing of an amount of about 150 mg or about 200 mg can be employed. In other embodiments involving the administration of a pharmaceutical composition comprising an anti-IL-6R antibody, once every six weeks dosing of an amount of about 150 mg or about 200 mg can be employed. In certain exemplary embodiments, the route of administration is subcutaneous.

The term “week” or “weeks” refers to a period of (n×7 days)±3 days, e.g., (n×7 days)±2 days, (n×7 days)±1 day, or (n×7 days), wherein “n” designates the number of weeks, e.g., 1, 2, 3, 4, 5, 6, 8, 12 or more.

The terms “initial dose,” “secondary doses,” and “tertiary doses,” refer to the temporal sequence of administration of the IL-6R antagonist. Thus, the “initial dose” is the dose that is administered at the beginning of the treatment regimen (also referred to as the “baseline dose” or “loading dose”); the “secondary doses” are the doses that are administered after the initial dose; and the “tertiary doses” are the doses that are administered after the secondary doses. The initial, secondary, and tertiary doses may all contain the same amount of IL-6R antagonist, or may differ from one another in terms of frequency of administration. In certain embodiments, however, the amount of IL-6R antagonist contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”). In one embodiment, the maintenance dose may be lower than the loading dose. In one embodiment, the secondary dose/maintenance dose may be equal to the initial dose/loading dose. For example, one or more initial doses/loading doses of 150 mg or 200 mg of IL-6R antagonist may be administered followed by secondary doses/maintenance doses of about 150 mg or about 200 mg, respectively. In one embodiment, a loading dose may be split, e.g., two or more doses administered at different time points, e.g., two loading doses wherein a second loading dose is administered two weeks after a first loading dose.

In some embodiments, the initial dose comprises 200 mg of the antibody or antigen-binding fragment thereof, and the one or more secondary doses comprises 200 mg of the antibody or antigen-binding fragment thereof administered every other week (every other week is used interchangeably with every two weeks, bi-weekly or q2w).

In one exemplary embodiment, each secondary and/or tertiary dose is administered 1 to 14 (e.g., 1, PA, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose” means, in a sequence of multiple administrations, the dose of IL-6R antagonist that is administered to a patient prior to the administration of the very next dose in the sequence with no intervening doses.

The methods may include administering to a patient any number of secondary and/or tertiary doses of an IL-6R antagonist. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

Methods comprising sequential administration of an IL-6R antagonist and a second therapeutic agent, to a patient to treat PMR or an associated condition are provided. In some embodiments, the methods comprise administering one or more doses of an IL-6R antagonist followed by one or more doses (e.g., 2, 3, 4, 5, 6, 7, 8, or more) of a second therapeutic agent. For example, one or more doses of about 150 mg to about 200 mg of the IL-6R antagonist may be administered after which one or more doses (e.g., 2, 3, 4, 5, 6, 7, 8, or more) of a second therapeutic agent (e.g., a corticosteroid) may be administered to treat, alleviate, reduce or ameliorate one or more symptoms of PMR. In some embodiments, the IL-6R antagonist is administered at one or more doses (e.g., 2, 3, 4, 5, 6, 7, 8, or more) resulting in an improvement in one or more PMR-associated parameters followed by the administration of a second therapeutic agent to prevent recurrence of at least one symptom of PMR. Alternative embodiments pertain to concomitant administration of an IL-6R antagonist and a second therapeutic agent. For example, one or more doses (e.g., 2, 3, 4, 5, 6, 7, 8, or more) of an IL-6R antagonist are administered and a second therapeutic agent is administered at a separate dosage at a similar or different frequency relative to the IL-6R antagonist. In some embodiments, the second therapeutic agent is administered before, after or concurrently with the IL-6R antagonist.

In certain embodiments, the IL-6R antagonist is administered every other week for 12 weeks, 14 weeks, 16 weeks, 18 weeks, 20 weeks, 22 weeks, 24 weeks, 26 weeks, 28 weeks, 30 weeks, 32 weeks, 34 weeks, 36 weeks, 38 weeks, 40 weeks, 42 weeks, 44 weeks, 46 weeks, 48 weeks or more. In specific embodiments, the IL-6R antagonist is administered for at least 14 weeks. In an exemplary embodiment, the IL-6R antagonist is administered for at least 52 weeks.

### Treatment Populations

The methods provided herein include administering to a subject in need thereof a therapeutic composition comprising an IL-6R antagonist. The expression “a subject in need thereof” means a human or non-human animal that exhibits one or more symptoms or indicia of PMR, or who has been diagnosed with PMR.

In a related embodiment, a “subject in need thereof” may be a subject who, prior to receiving an IL-6R antagonist, has been prescribed or is currently taking a steroid. In some embodiments, the subject may be a subject who, prior to receiving an IL-6R antagonist, has been prescribed or is currently taking a corticosteroid. In some embodiments, the subject is currently taking prednisone. For example, methods that comprise administering an IL-6R antagonist to a subject who has been taking a regular course of prednisone for eight or more weeks immediately preceding the administration of the IL-6R antagonist (such prior treatments are referred to herein as “background treatments”) are provided. In exemplary embodiments, the subject has been taking a regular course of prednisone at a dose of at least 7.5 mg/day and not more than 20 mg/day.

In yet other embodiments, the amount of the corticosteroid, such as the amount of prednisone, is gradually decreased prior to or after the start of IL-6R antagonist administration.

In another exemplary embodiment, a “subject in need thereof” has a diagnosis of PMR refractory to steroids prior to receiving the IL-6R antagonist. In some embodiments, the PMR symptoms of the subject persist despite treatment with steroids. In still another exemplary embodiment, a “subject in need thereof” has a diagnosis of PMR refractory to corticosteroids (e.g., prednisone) prior to receiving the IL-6R antagonist. In some embodiments, the PMR symptoms of the subject persist despite treatment with corticosteroids (e.g., prednisone).

In another exemplary embodiment, a “subject in need thereof” has a diagnosis of PMR refractory to steroid taper prior to receiving the IL-6R antagonist. In some embodiments, the subject experiences PMR flare when steroid taper is attempted. In an exemplary embodiment, the subject is refractory to corticosteroid (e.g., prednisone) taper and the subject experiences PMR flare when corticosteroid (e.g., prednisone) taper is attempted.

In another embodiment, a “subject in need thereof” is a subject whose PMR is not adequately controlled with steroids. In other embodiments, a “subject in need thereof” is a subject whose PMR is not adequately controlled with corticosteroids (e.g., prednisone). In some embodiments, a “subject in need thereof” is a subject for whom steroids are not advisable (i.e., the subject experiences adverse effects associated with steroids or is on a medication(s) that cannot be combined with steroid therapies. In some embodiments, a “subject in need thereof” is a subject for whom corticosteroids (e.g., prednisone) are not advisable (i.e., the subject experiences adverse effects associated with corticosteroids (e.g., prednisone) or is on a medication(s) that cannot be combined with corticosteroids (e.g., prednisone).

In a further exemplary embodiment, a “subject in need thereof” is a subject for whom steroids are not medically advisable (i.e., the subject has an allergy, a history of an adverse reaction, or other medical history wherein administration of steroids is not advisable). In an exemplary embodiment, the subject is a subject for whom corticosteroids (e.g., prednisone) are not medically advisable (i.e., the subject has an allergy, a history of an adverse reaction, or other medical history wherein administration of corticosteroids (e.g., prednisone) is not advisable).

In a further exemplary embodiment, a “subject in need thereof” is a subject who has had an inadequate response to one or more steroids. In an exemplary embodiment, the subject is a subject who has had an inadequate response to a corticosteroid (e.g., prednisone).

In a further exemplary embodiment, a “subject in need thereof” is a subject who cannot tolerate steroid taper. In an exemplary embodiment, the subject is a subject who cannot tolerate corticosteroid taper (e.g., prednisone taper).

In a further exemplary embodiment, a “subject in need thereof” is a subject who has had an inadequate response to steroid and/or who cannot tolerate steroid taper. In an exemplary embodiment, the subject is a subject who has had an inadequate response to corticosteroids (e.g., prednisone) and/or who cannot tolerate corticosteroid taper (e.g., prednisone taper).

In some embodiments, a “subject in need thereof” is at least 50 years old. In exemplary embodiments, the subject is older than 50 years old. In other exemplary embodiments, the subject has bilateral shoulder pain. In still other exemplary embodiments, the subject has a C-reactive protein (CRP) level of >10 mg/L and/or an erythrocyte sedimentation rate (ESR)>30 mm/hr. In some exemplary embodiments, the subject has morning stiffness. In other exemplary embodiments, the subject has an absence of joint involvement other than the shoulder joint. In still other exemplary embodiments, the subject has hip pain or limited range of motion. In some exemplary embodiments, the subject is seronegative for rheumatoid factor (RF) and anti-cyclic citrullinated peptide (anti-CCP). In other exemplary embodiments, the subject has at least one shoulder with subdeltoid bursitis and/or biceps tenosynovitis and/or posterior or axillary glenohumeral synovitis, and at least one hip with synovitis and/or trochanteric bursitis.

### Methods for Assessing Pharmacodynamic PMR-Associated Parameters

Methods for assessing one or more pharmacodynamic PMR-associated parameters in a subject in need thereof, caused by administration of a pharmaceutical composition comprising an IL-6R antagonist, are provided. A reduction in the incidence of PMR symptoms or an improvement in a PMR-associated PRO or ClinRO measure may correlate with an improvement in one or more pharmacodynamic PMR-associated parameters; however, such a correlation is not necessarily observed in all cases.

Examples of “pharmacodynamic PMR-associated parameters” include, for example, the following: (a) biomarker expression levels and (b) serum protein and RNA analysis. An “improvement in a pharmacodynamic PMR-associated parameter” means, for example, a decrease from baseline of one or more of levels of IL-6, IL6R, and C-reactive protein (CRP), or a decrease in erythrocyte sedimentation rate (ESR). As used herein, the term “baseline,” with regard to a pharmacodynamic PMR-associated parameter, means the numerical value of the pharmacodynamic PMR-associated parameter for a patient prior to or at the time of administration of a pharmaceutical composition described herein.

To assess a pharmacodynamic PMR-associated parameter, the parameter is quantified at baseline and at a time point after administration of the pharmaceutical composition. For example, a pharmacodynamic PMR-associated parameter may be measured at about day 1, about day 2, about day 3, day 4, about day 5, about day 6, about day 7, about day 8, about day 9, about day 10, about day 11, about day 12, about day 14, or at about week 3, about week 4, about week 5, about week 6, about week 7, about week 8, about week 9, about week 10, about week 11, about week 12, about week 13, about week 14, about week 15, about week 16, about week 17, about week 18, about week 19, about week 20, about week 21, about week 22, about week 23, about week 24, or longer, after the initial treatment with the pharmaceutical composition. The difference between the value of the parameter at a particular time point following initiation of treatment and the value of the parameter at baseline is used to establish whether there has been change, such as an “improvement”, in the pharmacodynamic PMR-associated parameter (e.g., an increase or decrease, as the case may be, depending on the specific parameter being measured).

In certain embodiments, administration of an IL-6R antagonist to a patient causes a change, such as a decrease or increase, in expression of a particular biomarker. PMR-associated biomarkers include, but are not limited to total IL-6, IL6R, and C-reactive protein (CRP). For example, administration of an IL-6R antagonist to a PMR patient can cause a decrease in IL-6, IL6R, or C-reactive protein (CRP) levels. The decrease can be detected at about week 1, about week 2, about week 3, about week 4, about week 5, or longer following administration of the IL-6R antagonist. Biomarker expression can be assayed by methods known in the art. For example, protein levels can be measured by ELISA (Enzyme Linked Immunosorbent Assay). RNA levels can be measured, for example, by reverse transcription coupled to polymerase chain reaction (RT-PCR).

Biomarker expression, as discussed above, can be assayed by detection of protein or RNA in serum. The serum samples can also be used to monitor additional protein or RNA biomarkers related to response to treatment with an IL-6R antagonist or IL-6 signaling. In some embodiments, RNA samples are used to determine RNA levels (non-genetic analysis), e.g., RNA levels of biomarkers; and in other embodiments, RNA samples are used for transcriptome sequencing (e.g., genetic analysis).

### Interleukin-6 Receptor Antagonists

The present disclosure includes methods that comprise administering to a subject an antibody, or an antigen-binding fragment thereof, that binds specifically to hIL-6R. As used herein, the term “hIL-6R” means a human cytokine receptor that specifically binds human interleukin-6 (IL-6). In certain embodiments, the antibody that is administered to the patient binds specifically to the extracellular domain of hIL-6R.

The term “antibody,” as used herein, refers to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In some embodiments, the FRs of the antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, and bivalent nanobodies), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody include: (i) V_{H}-C_{H}1; (ii) V_{H}-C_{H}2; (iii) V_{H}-C_{H}3; (iv) V_{H}—C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may in various embodiments consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody may in various embodiments comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

In certain embodiments, the antibody or antibody fragment for use in a method disclosed herein may be a monospecific antibody. In certain embodiments, the antibody or antibody fragment for use in a method disclosed herein may be a multispecific antibody, which may be specific for different epitopes of one target polypeptide or may contain antigen-binding domains specific for epitopes of more than one target polypeptide. An exemplary bi-specific antibody format that can be used in the context certain embodiments involves the use of a first immunoglobulin (Ig) C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise an Y96F modification (by IMGT; Y436F by EU). Further modifications that may be found within the second C_{H}3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies. Variations on the bi-specific antibody format described above are contemplated within the scope of certain embodiments. Any multispecific antibody format, including the exemplary bispecific antibody formats disclosed herein, may in various embodiments be adapted for use in the context of an antigen-binding fragment of an anti-IL-6R antibody using routine techniques available in the art.

The fully-human anti-IL-6R antibodies disclosed herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The present disclosure includes antibodies, and antigen-binding fragments thereof, which are derived from any of the amino acid sequences disclosed herein, wherein one or more amino acids within one or more framework and/or CDR regions are back-mutated to the corresponding germline residue(s) or to a conservative amino acid substitution (natural or non-natural) of the corresponding germline residue(s) (such sequence changes are referred to herein as “germline back-mutations”). A person of ordinary skill in the art, starting with the heavy and light chain variable region sequences disclosed herein, can easily produce numerous antibodies and antigen-binding fragments which comprise one or more individual germline back-mutations or combinations thereof. In certain embodiments, all of the framework residues and/or CDR residues within the V_{H} and/or V_{L} domains are mutated back to the germline sequence. In other embodiments, only certain residues are mutated back to the germline sequence, e.g., only the mutated residues found within the first 8 amino acids of FR1 or within the last 8 amino acids of FR4, or only the mutated residues found within CDR1, CDR2 or CDR3. Furthermore, included herein are antibodies that may contain any combination of two or more germline back-mutations within the framework and/or CDR regions, i.e., wherein certain individual residues are mutated back to the germline sequence while certain other residues that differ from the germline sequence are maintained. Once obtained, antibodies and antigen-binding fragments that contain one or more germline back-mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present disclosure.

The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

The term “human antibody,” as used herein, is intended to include antibodies having variable and constant regions derived from human germline immunoglobulin sequences. The human antibodies featured in the disclosure may in various embodiments nonetheless include amino acid residues not encoded by human germline immunoglobulin sequences (e.g., mutations introduced by random or site-specific mutagenesis in vitro or by somatic mutation in vivo), for example in the CDRs and in some embodiments CDR3. However, the term “human antibody,” as used herein, is not intended to include antibodies in which CDR sequences derived from the germline of another mammalian species, such as a mouse, have been grafted onto human framework sequences.

The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In an embodiment, an immunoglobulin molecule comprises a stable four chain construct of approximately 150-160 kDa in which the dimers are held together by an interchain heavy chain disulfide bond. In another embodiment, the dimers are not linked via inter-chain disulfide bonds and a molecule of about 75-80 kDa is formed composed of a covalently coupled light and heavy chain (half-antibody). In certain embodiments, these forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form to levels typically observed using a human IgG1 hinge. The instant disclosure encompasses in various embodiments antibodies having one or more mutations in the hinge, C_{H}2 or C_{H}3 region which may be desirable, for example, in production, to improve the yield of the desired antibody form.

An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody”. In various embodiments, the isolated antibody also includes an antibody in situ within a recombinant cell. In other embodiments, isolated antibodies are antibodies that have been subjected to at least one purification or isolation step. In various embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” IL-6R, as used herein, includes antibodies that bind IL-6R (e.g., human IL-6R) or portion thereof with a K_{D} of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or about 0.5 nM, as measured in a surface plasmon resonance assay. In some embodiments, the antibody binds IL-6R (e.g., human IL-6Rα) with a K_{D} of from about 0.1 nM to about 1000 nM or from about 1 nM to about 100 nM. In some embodiments, the antibody binds IL-6R (e.g., human IL-6Ra) with a K_{D} of from about 1 pM to about 100 pM or from about 40 pM to about 60 pM. Specific binding can also be characterized by a dissociation constant of at least about 1×10⁻⁶ M or smaller. In other embodiments, the dissociation constant is at least about 1×10⁻⁷ M, 1×10⁻⁸ M, or 1×10⁻⁹ M. An isolated antibody that specifically binds human IL-6R may, however, have cross-reactivity to other antigens, such as IL-6R molecules from other (non-human) species.

The term “surface plasmon resonance,” as used herein, refers to an optical phenomenon that allows for the analysis of real-time interactions by detection of alterations in protein concentrations within a biosensor matrix, for example using the BIACORE® system (Biacore Life Sciences division of GE Healthcare, Piscataway, NJ).

The term “K_{D},” as used herein, is intended to refer to the equilibrium dissociation constant of an antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The anti-IL-6R antibodies useful for the methods described herein may in various embodiments include one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present disclosure includes in various embodiments methods involving the use of antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). Numerous antibodies and antigen-binding fragments may be constructed which comprise one or more individual germline mutations or combinations thereof. In certain embodiments, all of the framework and/or CDR residues within the VH and/or VL domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies may contain any combination of two or more germline mutations within the framework and/or CDR regions, e.g., wherein certain individual residues are mutated to the corresponding residue of a certain germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. The use of antibodies and antigen-binding fragments obtained in this general manner are encompassed within the present disclosure.

The present disclosure also includes methods involving the use of anti-IL-6R antibodies comprising variants of any of the HCVR, LCVR, and/or CDR amino acid sequences disclosed herein having one or more conservative substitutions. For example, the present disclosure includes the use of anti-IL-6R antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

According to the present disclosure, the anti-IL-6R antibody, or antigen-binding fragment thereof, in various embodiments comprises a heavy chain variable region (HCVR), light chain variable region (LCVR), and/or complementarity determining regions (CDRs) comprising any of the amino acid sequences of the anti-IL-6R antibodies described in U.S. Pat. No. 7,582,298, incorporated herein by reference in its entirety. In certain embodiments, the anti-IL-6R antibody or antigen-binding fragment thereof comprises the heavy chain complementarity determining regions (HCDRs) of a HCVR comprising the amino acid sequence of SEQ ID NO: 1 and the light chain complementarity determining regions (LCDRs) of a LCVR comprising the amino acid sequence of SEQ ID NO: 2. According to certain embodiments, the anti-IL-6R antibody or antigen-binding fragment thereof comprises three HCDRs (i.e., HCDR1, HCDR2 and HCDR3) and three LCDRs (i.e., LCDR1, LCDR2 and LCDR3), wherein the HCDR1 comprises the amino acid sequence of SEQ ID NO: 3; the HCDR2 comprises the amino acid sequence of SEQ ID NO: 4; the HCDR3 comprises the amino acid sequence of SEQ ID NO: 5; the LCDR1 comprises the amino acid sequence of SEQ ID NO: 6; the LCDR2 comprises the amino acid sequence of SEQ ID NO: 7; and the LCDR3 comprises the amino acid sequence of SEQ ID NO: 8. In yet other embodiments, the anti-IL-6R antibody or antigen-binding fragment thereof comprises an HCVR comprising the amino acid sequence of SEQ ID NO: 1 and an LCVR comprising the amino acid sequence of SEQ ID NO: 2.

In another embodiment, the anti-IL-6R antibody or antigen-binding fragment thereof comprises a heavy chain comprising the amino acid sequence of SEQ ID NO: 9 and a light chain comprising the amino acid sequence of SEQ ID NO: 10. In some embodiments, the extracellular domain of hIL-6R comprises the amino acid sequence of SEQ ID NO: 11. According to certain exemplary embodiments, the methods of the present disclosure comprise the use of the anti-IL-6R antibody referred to and known in the art as sarilumab, or a bioequivalent thereof.

The amino acid sequence of SEQ ID NO: 1 is

EVQLVESGGGLVQPGRSLRLSCAAS [symbol] AMHWVRQAPGKGLEWVS
GI [symbol] [symbol] IGYADSVKGRFTISRDNAENSLFLQMNGLRAEDTALYYCAK [symbol]
WGQGTMVTVSS

(CDR sequences are highlighted: IMGT numbering in bold; Kabat numbering underlined; Chothia numbering italicized).

The amino acid sequence of SEQ ID NO: 2 is

DIQMTQSPSSVSASVGDRVTITCRAS [symbol] LAWYQQKPGKAPKLLIY [symbol]
SLESGVPSRFSGSGSGTDFTLTISSLQPEDFASYYC [symbol] F
GQGTKLEIK

(CDR sequences are highlighted: IMGT numbering in bold; Kabat numbering underlined; Chothia numbering italicized).

The amino acid sequence of SEQ ID NO: 3 is RFTFDDYA (CDR-H1 according to IMGT numbering).

The amino acid sequence of SEQ ID NO: 4 is ISWNSGRI (CDR-H2 according to IMGT numbering).

The amino acid sequence of SEQ ID NO: 5 is AKGRDSFDI (CDR-H3 according to IMGT numbering).

The amino acid sequence of SEQ ID NO: 6 is QGISSW (CDR-L1 according to IMGT numbering).

The amino acid sequence of SEQ ID NO: 7 is GAS (CDR-L2 according to IMGT numbering).

The amino acid sequence of SEQ ID NO: 8 is QQANSFPYT (CDR-L3 according to IMGT numbering).

The amino acid sequence of SEQ ID NO: 9 is

EVQLVESGGGLVQPGRSLRLSCAAS [symbol] AMHWVRQAPGKGLEWVS
GI [symbol] IGYADSVKGRFTISRDNAENSLFLQMNGLRAEDTALYYCAK [symbol]
WGQGTMVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLS
LSPGK

(CDR sequences are highlighted: IMGT numbering in bold; Kabat numbering underlined; Chothia numbering italicized).

The amino acid sequence of SEQ ID NO: 10 is

DIQMTQSPSSVSASVGDRVTITCRAS [symbol] LAWYQQKPGKAPKLLIY [symbol]
SLESGVPSRFSGSGSGTDFTLTISSLQPEDFASYYC [symbol] F
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

(CDR sequences are highlighted: IMGT numbering in bold; Kabat numbering underlined; Chothia numbering italicized).

The amino acid sequence of SEQ ID NO: 11 is

MVAVGCALLAALLAAPGAALAPRRCPAQEVARGVLTSLPGDSVTLTCPGV
EPEDNATVHWVLRKPAAGSHPSRWAGMGRRLLLRSVQLHDSGNYSCYRAG
RPAGTVHLLVDVPPEEPQLSCFRKSPLSNVVCEWGPRSTPSLTTKAVLLV
RKFQNSPAEDFQEPCQYSQESQKFSCQLAVPEGDSSFYIVSMCVASSVGS
KFSKTQTFQGCGILQPDPPANITVTAVARNPRWLSVTWQDPHSWNSSFYR
LRFELRYRAERSKTFTTWMVKDLQHHCVIHDAWSGLRHVVQLRAQEEFGQ
GEWSEWSPEAMGTPWTESRSPPAENEVSTPMQALTTNKDDDNILFRDSAN
ATSLPVQD.

According to the present disclosure, the anti-IL-6R antibody, or antigen-binding fragment thereof, in various embodiments comprises a heavy chain variable region (HCVR), light chain variable region (LCVR), and/or complementarity determining regions (CDRs) comprising any of the amino acid sequences of the anti-IL-6R antibodies described in U.S. Pat. No. 7,521,052, incorporated herein by reference in its entirety. The hybridoma cell line producing tocilizumab (TCZ) has been internationally deposited at International Patent Organism Depository (AIST Tsukuba Central 6, 1-1, Higashi 1-chome, Tsukuba-shi, Ibaraki Pref.) on the basis of Budapest Treaty as FERM BP-2998 on Jul. 12, 1989. In certain embodiments, the anti-IL-6R antibody or antigen-binding fragment thereof comprises the heavy chain complementarity determining regions (HCDRs) and or the light chain complementarity determining regions (LCDRs) of a HCVR comprising the amino acid sequence of SEQ ID NO: 13 and the light chain complementarity determining regions (LCDRs) of a LCVR comprising the amino acid sequence of SEQ ID NO: 12. According to certain embodiments, the anti-IL-6R antibody or antigen-binding fragment thereof comprises three HCDRs (i.e., HCDR1, HCDR2 and HCDR3) and three LCDRs (i.e., LCDR1, LCDR2 and LCDR3), wherein the HCDR1 comprises the amino acid sequence of SEQ ID NO: 17; the HCDR2 comprises the amino acid sequence of SEQ ID NO: 18; the HCDR3 comprises the amino acid sequence of SEQ ID NO: 19; the LCDR1 comprises the amino acid sequence of SEQ ID NO: 14; the LCDR2 comprises the amino acid sequence of SEQ ID NO: 15; and the LCDR3 comprises the amino acid sequence of SEQ ID NO: 16. In various embodiments, the anti-IL-6R antibody or antigen-binding fragment thereof comprises an heavy chain comprising the amino acid sequence of SEQ ID NO: 13 and an light chain comprising the amino acid sequence of SEQ ID NO: 12.

In another embodiment, the anti-IL-6R antibody or antigen-binding fragment thereof comprises a heavy chain comprising the amino acid sequence of the heavy chain of TCZ and a light chain comprising the amino acid sequence of the light chain of TCZ. In some embodiments, the extracellular domain of hIL-6R comprises the amino acid sequence of the extracellular domain of TCZ. According to certain exemplary embodiments, the methods of the present disclosure comprise the use of the anti-IL-6R antibody referred to and known in the art as tocilizumab, or a bioequivalent thereof.

The amino acid sequence of SEQ ID NO: 12 is

DIQMTQSPSSLSASVGDRVTITCRASQDISSYLNWYQQKPGKAPKLLIYY
TSRLHSGVPSRFSGSGSGTDFTFTISSLQPEDIATYYCQQGNTLPYTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

The amino acid sequence of SEQ ID NO: 13 is

VQLQESGPGLVRPSQTLSLTCTVSGYSITSDHAWSWVRQPPGRGLEWIGY
ISYSGITTYNPSLKSRVTMLRDTSKNQFSLRLSSVTAADTAVYYCARSLA
RTTAMDYWGQGSLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPG.
The amino acid sequence of SEQ ID NO: 14 is
RASQDISSYLN.
The amino acid sequence of SEQ ID NO: 15 is
YTSRLHS.
The amino acid sequence of SEQ ID NO: 16 is
QQGNTLPYT.
The amino acid sequence of SEQ ID NO: 17 is
SDHAWS.
The amino acid sequence of SEQ ID NO: 18 is
YISYSGITTYNPSLK.
The amino acid sequence of SEQ ID NO: 19 is
SLARTTAMDY.

The term “bioequivalent” as used herein, refers to a molecule having similar bioavailability (rate and extent of availability) after administration at the same molar dose and under similar conditions (e.g., same route of administration), such that the effect, with respect to both efficacy and safety, can be expected to be essentially same as the comparator molecule. Two pharmaceutical compositions comprising an anti-IL-6R antibody are bioequivalent if they are pharmaceutically equivalent, meaning they contain the same amount of active ingredient (e.g., IL-6R antibody), in the same dosage form, for the same route of administration and meeting the same or comparable standards. Bioequivalence can be determined, for example, by an in vivo study comparing a pharmacokinetic parameter for the two compositions. Parameters commonly used in bioequivalence studies include peak plasma concentration (Cmax) and area under the plasma drug concentration time curve (AUC).

The disclosure in certain embodiments relates to methods comprising administering to the subject an antibody which comprises the heavy chain variable region comprising sequence SEQ ID NO: 1 and the light chain variable region comprising sequence SEQ ID NO: 2.

The disclosure provides pharmaceutical compositions comprising such antibody, and methods of using these compositions.

The antibody in various embodiments comprises the heavy chain variable region comprising sequence SEQ ID NO: 1 and the light chain variable region comprising sequence SEQ ID NO: 2 is an antibody that specifically binds human interleukin-6 receptor (hIL-6R). See international publication number WO2007/143168, incorporated herein by reference in its entirety. In one embodiment, the antibody comprises the heavy chain variable region comprising sequence SEQ ID NO: 9 and the light chain variable region comprising sequence SEQ ID NO: 10. In various embodiments, the antibody is sarilumab. Sarilumab is also known by the tradename KEVZARA®.

### Example 1. A Randomized, Double-Blind, Placebo-Controlled Study to Evaluate the Efficacy and Safety of Sarilumab in Patients with Polymyalgia Rheumatica (NCT03600818, EFC15160, Phase 3)

### Material and Methods

### Number of Study Center(s) and Countries

A total of 70 active centers enrolled at least 1 participant and of these 60 sites randomized at least 1 participant. The sites were located in 17 countries (Australia, Argentina, Belgium, Canada, Estonia, France, Germany, Hungary, Israel, Italy, Japan, Netherlands, Russia, Spain, Switzerland, United Kingdom, and United States).

### Rationale

Study EFC15160 was designed as a 52-week, double-blind, placebo-controlled, randomized, study to evaluate the efficacy and safety of sarilumab in patients with active polymyalgia rheumatica (PMR). The 52-week study treatment duration reflected the usual duration and standard of care therapy required to ensure sustained remission for the majority of patients with PMR (hence, the primary endpoint of sustained remission was at week 52). The ability to taper off of CS rapidly in 14 weeks (as opposed to the usual tapering regimen of 1 year or more) after initiating therapy while maintaining disease remission represents an important clinically meaningful benefit over usual care.

### Objectives

### Primary Objective

To evaluate the efficacy of sarilumab in patients with PMR as assessed by the proportion of patients with sustained remission at Week 52 for sarilumab with a 14-week corticosteroids (CS) tapering regimen as compared to placebo with a 52-week CS tapering regimen.

### Secondary Objectives

To demonstrate the efficacy of sarilumab (with 14-week taper of CS) compared to placebo (with 52-week taper of CS) in patients with PMR with regards to: clinical responses (such as components of sustained remission, disease remission rates, time to first disease flare) over time and cumulative corticosteroid (including prednisone) exposure.

To assess the safety (including immunogenicity) and tolerability of sarilumab in patients with PMR.

To measure sarilumab concentrations from serum of patients with PMR.

To assess the effect of sarilumab on reducing glucocorticoid toxicity as measured by the composite glucocorticoid toxicity index (GTI) questionnaire.

### Exploratory Objectives

To assess the effect of sarilumab on physician assessment of disease activity as measured by a visual analogue scale (MD-VAS).

To assess the effect of sarilumab on a variety of PRO concepts, including fatigue (as measured by FACIT-fatigue scale), health status (as measured by EQ-5D-3L and SF-36v2), physical function (as measured by HAQ-DI, pain (as measured via HAQ-DI by a visual analogue scale [VAS]) and patient assessment of disease activity (as measured via HAQ-DI by a VAS).

To assess the impact of ESR/CRP levels on remission status.

To characterize the disease activity of PMR patients while on steroid taper or sarilumab treatment in a subset of patients using comprehensive approaches to evaluate circulating immune cell types.

To characterize the disease activity of PMR patients while on steroid taper or sarilumab treatment by evaluating circulating proteins, genetics and gene expression in patients who consent for this optional part of the study.

### Study Design

This was a multicenter, randomized, double-blind, placebo-controlled 52-week, Phase 3 study, evaluating the efficacy and safety of sarilumab in patients with active PMR.

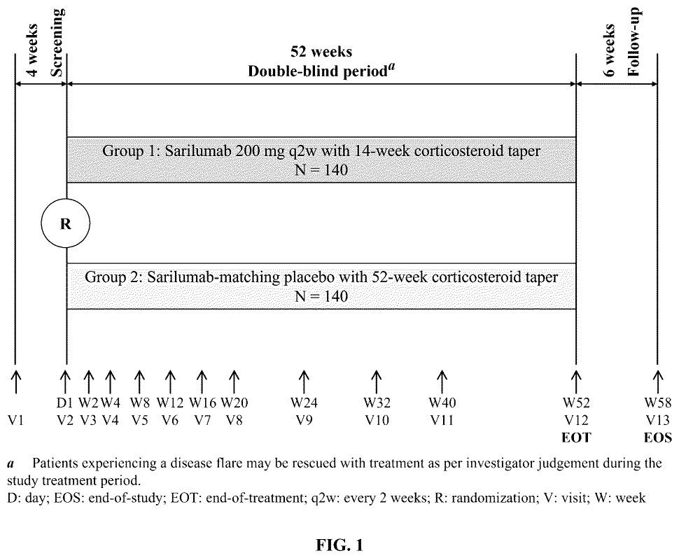

As shown in FIG. 1, patients with active PMR who met the entry criteria were randomized into the following 2 parallel treatment groups with sarilumab 200 mg or placebo plus protocol-defined CS tapering regimens of either 14 or 52 weeks in a 1:1 ratio: group 1: sarilumab 200 mg q2w with a 14-week taper of CS and group 2: sarilumab matching placebo q2w with a 52-week taper of CS. All patients received sarilumab 200 mg or placebo for 52-weeks.

All patients received prednisone treatment (CS taper) with a different regimen depending on the assigned group. Prior to randomization and initiation of study treatment, corticosteroid therapy was optimized to minimize the risk of serious adverse events associated with tapering of corticosteroids. The initial dose of prednisone for both groups was 15 mg/day for the first 2 weeks after randomization and then prednisone and/or prednisone matched placebo was given to patients in order to ensure the double-blind CS tapering regimen as defined below.

Group 1: From Week 2 to Week 13, patients received gradually decreasing dose levels of prednisone (prednisone or combination of prednisone and placebo to prednisone). From Week 14 onwards, patients without flare received prednisone matching placebo.

Group 2: From Week 2 to Week 51, patients received gradually decreasing dose levels of prednisone (prednisone or combination of prednisone and placebo).

At each site visit, the patients' disease was assessed to determine whether the patient could adhere to the protocol defined prednisone taper schedule. During the initial 12 weeks of prednisone taper, treatment for one flare was permitted if it could be successfully treated with a low dose (≤5 mg/day) prednisone add-on taper regimen (completed prior to Week 12) and provided that all other sustained remission parameters were met.

For patients who experienced a disease flare and needed rescue therapy (such as CS) as per investigator judgment, during the course of the study, they could continue administration of sarilumab or matching placebo in a double-blinded fashion for the full duration of the 52-week treatment period only if corticosteroids were used as rescue therapy. Treatment with non-biological immunosuppressive drugs (such as alkylating agents, hydroxychloroquine, CsA, MMF, AZA, etc). was not permitted during the course of the study, unless used for the purpose of rescue therapy.

During the course of study, for patients in need of rescue therapy as per investigator judgment, corticosteroids were the agent of first choice. Patients could continue SC administration of sarilumab or matching placebo only if CS is used as rescue therapy. If the patients remain symptomatic despite CS rescue therapy, then other treatment options including non-biological immunosuppressive drugs could be used (patient must have symptomatic PMR disease) and the patient was discontinued from the study treatment and considered a non-responder.

The total duration of study participation for each patient was up to 62 weeks: up to 4 weeks of a screening period, 52-week treatment period, and a 6-week post treatment follow up period. The last patient last visit occurred when the last patient completed the 52-week double blind treatment period and the follow up period of 6 weeks (Visit 13/end of study (EOS)). The end of the clinical trial was defined as the last patient's last visit. The schedule of events is shown in Table 1, below.

TABLE 1
Schedule of events

| | VISIT | | | | | | | | | | | | |
|---|---|---|---|---|---|---|---|---|---|---|---|---|---|
| | | | | | | | | | | | | V12 | V13 |
| | V1 | V2 | V3 | V4 | V5 | V6 | V7 | V8 | V9 | V10 | V11 | (EOT) | (EOS) |
| | DAY | | | | | | | | | | | | |
| | D-28 | | D15 | D29 | D57 | D85 | D113 | D141 | D169 | D225 | D281 | D365 | D407 |
| | to D-1 | D1 | (±3) | (±3) | (±3) | (±3) | (±3) | (±3) | (±3) | (±3) | (±3) | (±3) | (±3) |
| | WEEK | | | | | | | | | | | | |
| | | W0 | W2 | W4 | W8 | W12 | W16 | W20 | W24 | W32 | W40 | W52 | W58 |
| Eligibility | | | | | | | | | | | | | |
| Written informed consent | X | | | | | | | | | | | | |
| Inclusion/exclusion criteria^{t} | X | X | | | | | | | | | | | |
| Ultrasound central reading^{v} | X | | | | | | | | | | | | |
| Patient demography | X | | | | | | | | | | | | |
| Medical/surgical/smoking- | | | | | | | | | | | | | |
| alcohol history | | | | | | | | | | | | | |
| Prior medication history | X | | | | | | | | | | | | |
| Family cardiovascular | X | | | | | | | | | | | | |
| history | | | | | | | | | | | | | |
| Full physical examination | X | | | | | | | | | | | X | |
| Targeted physical | | X | | | | X | | | X | | X | | |
| examination^{a} | | | | | | | | | | | | | |
| Confirm eligibility | | X | | | | | | | | | | | |
| Randomization | | X | | | | | | | | | | | |
| Call IRT | X | X | | X | X | X | X | X | X | X | X | X | X |
| Treatment | | | | | | | | | | | | | |
| Initial treatment kit | | X | | | | | | | | | | | |
| assignment (IRT) | | | | | | | | | | | | | |
| IMP administration^{b} | | X^{w} | X | X | X | X | X | X | X^{s} | X^{s} | X^{s} | | |
| Concomitant medication | X^{u} | X | X | X | X | X | X | X | X | X | X | X | X |
| review | | | | | | | | | | | | | |
| Dispense patient diary | | X | X | X | X | X | X | X | X | X | X | | |
| Compliance/review patient | | | X | X | X | X | X | X | X | X | X | X | |
| diary | | | | | | | | | | | | | |
| Vital signs | | | | | | | | | | | | | |
| Temperature, heart rate, | X | X | X | X | X | X | X | X | X | X | X | X | |
| blood pressure | | | | | | | | | | | | | |
| Weight | X | X | | | | X | | | X | | X | X | |
| Height | X | | | | | | | | | | | | |
| Efficacy | | | | | | | | | | | | | |
| PMR clinical assessments | X | X | X | X | X | X | X | X | X | X | X | X | X |
| (including disease flare) | | | | | | | | | | | | | |
| Physician global | | X | | | | X | | | X | | | X | |
| assessment (MD-VAS) | | | | | | | | | | | | | |
| Patient reported outcomes^{c} | | X | | | | X | | | X | | | X | |
| Glucocorticoid toxicity | | X | | | | X | | | X | | X | X | |
| index (excl. bone density | | | | | | | | | | | | | |
| assessment) | | | | | | | | | | | | | |
| Bone density assessment^{d} | | X | | | | | | | | | | X | |
| Safety | | | | | | | | | | | | | |
| AE/SAE recording | | | | | | | | | | | | | |
| Tuberculosis assessment | X | X | X | X | X | X | X | X | X | X | X | X | X |
| QuantiFERON ® | X | | | | | | | | | | | | |
| Chest X-ray^{e} | X | | | | | | | | | | | | |
| Laboratory testing | | | | | | | | | | | | | |
| Hematology^{f} | X | X | | X | | X | | | X | | X | X | |
| Chemistry^{g} | X | X | | X | | X | | | X | | X | X | |
| ANA^{h} | | X | | | | | | | | | | X | |
| Fasting lipidsⁱ and fasting | X | | | X | | X | | | X | | X | X | |
| glucose/insulin^{j} | | | | | | | | | | | | | |
| HbA1c | X | | | X | | X | | | X | | X | X | |
| CRP^{k} | X | X | X | X | X | X | X | X | X | X | X | X | |
| ESR^{k} | X | X | X | X | X | X | X | X | X | X | X | X | |
| Urinalysis^{l} | X | | | | | | | | | | | | |
| Virology^{m} | X | | | | | | | | | | | | |
| Serum pregnancy testⁿ | X | | | | | | | | | | | | |
| Urine pregnancy testⁿ | | X | | X | X | X | X | X | X | X | X | X | |
| 12-lead ECG | X | | | | | | | | | | | | |
| Serum sarilumab^{j} | | X | X | X | | X | X | | x^{r} | | | X | X |
| Antibodies to | | X | | | | X | | | X | | | X | X |
| sarilumab/ADA^{j} | | | | | | | | | | | | | |
| Genotyping and | | | | | | | | | | | | | |
| biomarkers^{j} | | | | | | | | | | | | | |
| Biomarkers-IL-6 and sIL- | | X | X | | | X | | | X | | | X | |
| 6R | | | | | | | | | | | | | |
| Immune cell phenotyping | | X | | X | | | | | X | | | | |
| (whole blood)^{o} | | | | | | | | | | | | | |
| Future use samples (serum | | X | X | | | X | | | X | | | X | |
| and plasma)^{p} - Optional | | | | | | | | | | | | | |
| DNA^{q} - Optional | | X | | | | | | | | | | | |
| RNA^{q} - Optional | | X | X | | | | | | | | | | |

AE = Adverse event;
D = Day;
DNA = Deoxyribonucleic acid
ECG = Electrocardiogram;
EOT = End of treatment;
EOS = End of study;
EQ-5D = EuroQol;
ESR = erythrocyte sedimentation rate;
EUL = elevate upper limb;
FACIT-Fatigue = Functional assessment of chronic illness therapy fatigue scale;
GCA = Giant cell arteritis;
GTI = Glucocorticoid toxicity index;
HbA1c = Hemoglobin A1c
HBsAg = Hepatitis B surface antigen;
HBcore Ab = Hepatitis B core antibodies;
HCV = Hepatitis C virus;
CRP = C-reactive protein;
IL = Interleukin;
IMP = Investigational medicinal product;
IRT = Interactive voice response system;
MD-VAS = Physician global assessment of disease activity-visual analog scale;
Pain VAS = Pain visual analog scale;
Pt-VAS = Patient global assessment of disease activity- visual analog scale;
RNA = Ribonucleic acid;
SAE = Serious adverse event;
SF-36v2 = Short form 36v2;
V = Visit; Wk = Week.
^{a}Targeted physical examination: head, eyes, ears, neck and throat, skin, respiratory, cardiovascular, neurologic, lymphatic examinations and abdominal examination.
^{b}Last administration of sarilumab is at Week 50. Patients will be monitored for at least 30 minutes or up to 2 hours as per country specific requirements after each dose of SC IMP for any signs or symptoms of a hypersensitivity reaction.
^{c}Patient Reported Outcomes include EQ-5D-3L, FACIT-Fatigue, SF-36v2, HAQ-DI
^{d}Bone Mineral Density assessment will be performed at the baseline (Visit 2) and Week 52 (Visit 12) using a DXA scan. The scan can be performed within ±14 days of Visit 2 and within -14 days of Visit 12 and needs to include the lumbosacral and femoral neck regions. However, the baseline visit DXA scan is not required if there is one available within 12 weeks of baseline.
^{e}Chest X-ray is required during the screening period if no chest imaging (X-ray, CT, MRI) is available within the previous 12 weeks of VI that clearly documents the exclusion of TB or if it does not follow the local guidelines and requirements for active screening of TB. In countries for which a specific approval procedure for the x-ray is required by a different committee than the local EC/IRB, a chest MRI between V1 and V2 can be performed.
^{f} Hematology (blood should be drawn before drug administration): Hemoglobin, hematocrit, red blood cell (RBC) count and morphology (if RBC count is abnormal), white blood cell (WBC) differential, platelet count, absolute neutrophil count (ANC).
^{g}Chemistry(blood should be drawn before drug administration): Sodium, potassium, chloride, bicarbonate, blood urea nitrogen (BUN), creatinine and creatinine clearance, calcium, phosphate, total protein, albumin, alanine aminotransferase (ALT) (SGPT), aspartate aminotransferase (AST) (SGOT), alkaline phosphatase (ALP), total bilirubin, conjugated bilirubin, unconjugated bilirubin, lactate dehydrogenase (LDH), uric acid
^{h}Anti-nuclear antibody (ANA) was collected at Visit 2 and EOT visits only.
ⁱLipids (blood should be drawn before drug administration): Triglycerides (TG), total cholesterol, high density lipoprotein (HDL) cholesterol, low density lipoprotein (LDL) cholesterol. Fasting is defined as having no food or liquid intake (except water/ice) for six hours or more.
jBlood should be drawn before drug administration. Fasting is defined as having no food or liquid intake (except water/ice) for six hours or more.
^{k}CRP and ESR results will be blinded to both Investigator and Sponsor (except screening and baseline). ESR kits will be provided by the central laboratory while the test will be performed locally at the site; results will be blinded to Investigator and staff directly involved in management of study patient except the safety assessor.
^{l}Urinalysis dipstick: specific gravity, pH, glucose, blood, protein, nitrites, leukocytes, ketones, urobilinogen and bilirubin (by dipstick) at screening visit only. If any parameter on the dipstick is abnormal, a urine sample should be sent to the central laboratory for testing. If positive for proteins, microscopic analysis is performed by central laboratory.
^{m}Human immunodeficiency virus antibodies - if required locally, the locally provided consent for the required HIV screening test will be collected; Hepatitis B: Hep B surface antigen, total Hep B core antibody, Hep B surface antibody, and Hep B viral DNA (if necessary); Hepatitis C: HCV-antibody.
ⁿIn women of child-bearing potential.
^{o}Immune Cell Phenotyping (Whole Blood): Approximately 40 patients from each of the two treatment arms will be selected for this whole blood draw and immune cell phenotyping analysis.
^{p}A separate Future Use Samples Informed Consent had to be obtained before any sampling. Both serum and plasma will be drawn and the samples will be used for future analysis (eg, circulating proteins).
^{q}A separate Pharmacogenetic Research Informed Consent for collecting and sequencing DNA and RNA samples has to be obtained before any sampling. One DNA (at baseline or any treatment or follow up visit) and RNA sample for sequencing sampling time point at baseline and pre-dose (V3) are needed.
^{r}Additional sample is to be drawn 4-7 days after Week 24 dosing.
^{s}Since the visit interval exceeds 4 weeks, interim shipments of IMP to patients home could be performed using direct to patient (DTP) shipping in order to provide the patient with only 4 weeks IMP at a time in order to minimize compliance errors.
^{t}If the ultrasound is employed in the diagnosis of PMR, then the ultrasound images need to be submitted to the central reader for confirmation that they fulfill the ultrasound part of the diagnostic criteria for PMR.
^{u}For patients who were on >15 mg/day (but not exceeding 20 mg/day) of prednisone at screening and during the screening period, the Investigator should judiciously taper the prednisone down to 15 mg/day prior to randomization in order to prevent a disease flare upon entering the study at 15 mg/day of prednisone.
^{v}If ultrasound is being used as a diagnostic tool of PMR, the image needs to be submitted to the central reader for confirmation of eligibility.
^{w}IMP training. Prior to the first dose of IMP, provided instructions on preparation and self-injection of the pre-filled syringes and the use of the weekly blister packs of prednisone.

The study terminated early due to the protracted recruitment timeline exacerbated by the COVID-19 pandemic. Approximately 118 patients who satisfied the eligibility criteria were enrolled and randomized into 2 parallel arms to receive either sarilumab 200 mg q2w with 14-weeks prednisone taper (Group 1) or sarilumab matching placebo with 52-weeks prednisone taper (Group 2) in the ratio of 1:1. As shown in Table 2, 117 of 118 patients that were randomized received treatment.

TABLE 2
Patient disposition

| | Sarilumab 200 mg | | Placebo + | |
|---|---|---|---|---|
| | Q2W + 14-week | | 52-week GC taper | |
| Patients, n (%) | GC taper (N = 60) | | (N = 58) | |
| Randomized and not treated | 1 | (1.7) | 0 | |
| Randomized and treated | 59 | (98.3) | 58 | (100) |
| Completed the study | 42 | (70.0) | 36 | (62.1) |
| treatment period | | | | |
| Discontinued from study | 17 | (28.3) | 22 | (37.9) |
| treatment period | | | | |
| Adverse events | 7 | (11.7) | 4 | (6.9) |
| Lack of efficacy | 4 | (6.7) | 9 | (15.5) |
| Withdrawal by patient | 3 | (5.0) | 4 | (6.9) |
| Other | 3 | (5.0) | 5 | (8.6) |
| Related to COVID-19 | 0 | | 0 | |
| Not related to COVID-19* | 3 | (5.0) | 5 | (8.6) |

### Study Committees

If ultrasound was used to diagnose PMR and determine patient eligibility, the ultrasound images was centrally reviewed by an expert rheumatologist (from a group of expert rheumatologists) specialized in the performance and interpretation of diagnostic ultrasounds of the shoulders and hips in order to confirm the diagnosis. Additionally, the same group of expert rheumatologists served to help certify the sites who wished to have the option of using ultrasound in the diagnosis of PMR for their patients.

### Inclusion Criteria

Signed written informed consent.

Diagnosis of PMR according to the European League against Rheumatism/American College of Rheumatology (EULAR/ACR) classification criteria as follows (must satisfy all criteria): a) Age≥50 years at time of diagnosis, b) bilateral shoulder pain, and elevated acute phase reactants (CRP>10 mg/L and/or ESR>30 mm/hr).

AND one of the following:
a score≥4 at the time of diagnosis based on the following (without ultrasound): duration of morning stiffness>45 minutes: 2 points; hip pain or limited range of motion: 1 point; seronegative for rheumatoid factor (RF) and anti-cyclic citrullinated peptide (anti-CCP): 2 points; and absence of other joint involvement: 1 point; or
a score ≥5 at the time of diagnosis based on the following (with ultrasound): duration of morning stiffness >45 minutes: 2 points; hip pain or limited range of motion: 1 point; seronegative for RF and anti-CCP: 2 points; absence of other joint involvement: 1 point; at least one shoulder with subdeltoid bursitis and/or biceps tenosynovitis and/or glenohumeral synovitis (either posterior or axillary) and at least one hip with synovitis and/or trochanteric bursitis: 1 point (confirmed with ultrasound); and both shoulders with subdeltoid bursitis, biceps tenosynovitis or glenohumeral synovitis: 1 point (confirmed with ultrasound).

Patient must be on prednisone of at least 7.5 mg/day (or equivalent) and not exceeding mg/day at screening and during the screening period.

Patient is willing and able to receive prednisone of 15 mg/day at randomization.

Patient must have a history of being treated for at least 8 weeks with prednisone ≥10 mg/day or equivalent.

Patient must have had at least one episode of unequivocal PMR flare while attempting to taper prednisone at a dose that is ≥7.5 mg/day (or equivalent) within the past 12 weeks prior to screening. Unequivocal symptoms of PMR flare are defined as shoulder and/or hip girdle pain associated with inflammatory stiffness.

Patient must have ESR≥30 mm/hr or CRP≥10 mg/L associated with PMR disease activity within 12 weeks prior to screening.

### Exclusion Criteria

Patients who met all the above inclusion criteria were screened for the following exclusion criteria:

Diagnosis of Giant Cell Arteritis (GCA) (e.g., persistent or recurrent localized headache, temporal artery or scalp tenderness, jaw claudication, extremity claudication, blurry or loss of vision, symptoms of stroke).

Concurrent rheumatoid arthritis or other inflammatory arthritis or other connective tissue diseases, such as but not limited to systemic lupus erythematosus, systemic sclerosis, vasculitis, myositis, mixed connective tissue disease, and ankylosing spondylitis.

Concurrent diagnosis of rhabdomyolysis or neuropathic muscular diseases.

Concurrent diagnosis of active fibromyalgia.

Inadequately treated hypothyroidism.

Organ transplant recipient.

Any prior (within the defined periods below) or concurrent use of immunosuppressive therapies including but not limited to the following: Janus kinase (JAK) inhibitor (e.g., tofacitinib) within 4 weeks of baseline; cell-depletion agents (e.g., anti CD20) without evidence of recovery of B cells to baseline level; anakinra within 1 week of baseline; abatacept within 8 weeks of baseline; tumor necrosis factor (TNF) inhibitors within 2-8 weeks (etanercept within 2 weeks, infliximab, certolizumab, golimumab, or adalimumab within 8 weeks), or after at least 5 half-lives have elapsed, whichever is longer; alkylating agents including cyclophosphamide (CYC) within 6 months of baseline; and cyclosporine (CsA), azathioprine (AZA) or mycophenolate mofetil (MMF) or leflunomide within 4 weeks of baseline.

Therapeutic failure, including inadequate response or intolerance, or contraindication, to biological IL-6 antagonist (prior IL-6 antagonist treatment that was terminated for reasons unrelated to therapeutic failure at least 3 months before baseline is not exclusionary).

Unstable methotrexate (MTX) dose and/or MTX dose>15 mg/week within 3 months of baseline.

Concurrent use of systemic CS for conditions other than PMR.

Participation in any clinical research study that evaluated an investigational drug or therapy within 5 half-lives or 60 days of the Screening Visit, whichever is longer.

History of alcohol or drug abuse within 5 years prior to the screening visit.

Patient who withdraws consent during the screening period (following signing of the informed consent form).

Unable or unwilling to complete the patient-reported outcome (PRO) questionnaires.

Patient who meets any of the following conditions/situations: patients with short life expectancy; conditions/concomitant diseases making patients non-evaluable for the efficacy endpoints (e.g., patients with chronic pain); patient is the Investigator or any Subinvestigator, Research Assistant, Pharmacist, Study Coordinator, other staff or relative thereof, directly involved in the conduct of the study, or, as applicable to employee of site/Investigator or Sponsor; uncooperative, or any condition, that could make the patient potentially noncompliant to the study procedures, etc., and individuals who are institutionalized due to regulatory or legal order.

Pregnant or breastfeeding woman.

Woman of childbearing potential (WOCBP) not protected by highly-effective contraceptive method(s) of birth control and/or who are unwilling or unable to be tested for pregnancy.

### Exclusion Related to Tuberculosis (TB)

Active TB or a history of incompletely treated TB regardless of screening Quantiferon result.

Quantiferon positive patients (no active disease) are excluded from the study unless the following conditions are met: patients with a history of prior documented completed chemoprophylaxis for latent tuberculosis infection (e.g., acceptable treatments include 9 months of isoniazid 300 mg oral daily or equivalent proven regimen per local guidelines) or treatment of active tuberculosis infection (TBI) who has obtained consultation with a specialist to rule out active TBI or the need to receive further treatment; patients with no prior history of chemoprophylaxis for latent TBI or treatment for active TBI but have obtained consultation with a specialist to initiate an appropriate regimen of chemoprophylaxis, based on local epidemiology and applicable guidelines and have demonstrated compliance and tolerated treatment for ≥1 month; or consultation with and prior approval from sponsor are required in either of the aforementioned scenarios.

Clinically significant abnormality consistent with prior/active TB infection based upon chest radiograph with at least posterior-anterior view. Additional lateral view is recommended but not required.

Suspected extra-pulmonary TB infection regardless of screening Quantiferon result.

Patients at high risk of contracting TB, such as close contact with individuals with active or latent TB.

Patient who received Bacille Calmette Guerin vaccination within 12 months prior to screening.

### Other Exclusion Criteria

Patients with a history of invasive opportunistic infections, including but not limited to histoplasmosis, listeriosis, coccidioidomycosis, candidiasis, pneumocystis jirovecii, aspergillosis despite resolution or John Cunningham virus (progressive multifocal leukoencephalopathy).

Patients with fever (>38° C.) associated with infection, or chronic, persistent, or recurring infection(s) requiring active treatment with antibiotics, antivirals, or antifungals within 4 weeks prior to the screening visit or other frequent recurrent infections deemed unacceptable as per Investigator judgment.

Patients with uncontrolled diabetes mellitus, defined as glycosylated hemoglobin (HbA1c)≥9% at the screening visit.

Patients with non-healed or healing skin ulcers.

Patients who received any live, attenuated vaccine within 3 months prior to the baseline visit, such as varicella-zoster, oral polio or rubella vaccines.

Patients who are positive for hepatitis B surface antigen (HBsAg) or are positive for total hepatitis B core antibody (HBcAb) with negative hepatitis B surface antibody (HBsAb) or are positive for both HBcAb and HBsAb with presence of HBV DNA at screening.

Patients who are positive for hepatitis C antibody (HCV Ab).

Patients who are positive for human immunodeficiency virus (HIV) antibody test at screening or who previously had a positive HIV antibody test, or who are suspected to be positive for HIV.

Patients with a history of recurrent herpes zoster or active herpes zoster.

Patients with a history of prior articular or prosthetic joint infection.

Prior or current history of malignancy, including lymphoproliferative diseases, other than adequately treated carcinoma in-situ of the cervix, non-metastatic squamous cell or basal cell carcinoma of the skin, within 5 years prior to the baseline visit.

Prior or current history of other significant concomitant illness(es) that, according to Investigator's judgment, would adversely affect the patient's participation in the study. These include, but are not limited to, cardiovascular (including Stage III or IV cardiac failure according to the New York Heart Association classification), renal, neurological (including demyelinating disease), active infectious diseases, endocrinological, gastrointestinal, hepato-biliary, metabolic, pulmonary, non-malignant lymphoproliferative disease or other lymphatic disease(s).

Patients who have had surgery within 4 weeks prior to the screening visit or with planned surgery during the course of the study.

Patients with a history of a systemic hypersensitivity reaction, other than localized injection site reaction, to any biologic drug and known hypersensitivity to any constituent of the sarilumab product.

Patients with any of the following laboratory abnormalities at the screening visit: hemoglobin <8.5 g/dL; white blood cells <3000/mm³; neutrophils <2000/mm³; platelet count <150,000 cells/mm³; aspartate aminotransferase (AST) or alanine aminotransferase (ALT)>1.5×upper limits of normal (ULN); bilirubin (total)>ULN, unless documented Gilbert's disease diagnosed by genetic testing; presence of severe uncontrolled hypercholesterolemia (>350 mg/dL, 9.1 mmol/L) or hypertriglyceridemia (>500 mg/dL, 5.6 mmol/L); and patients with a calculated creatinine clearance <30 mL/minute (using Cockroft-Gault formula).

Patients with a history of inflammatory bowel disease or severe diverticulitis or previous gastrointestinal perforation.

### Study Treatments

### Investigational Medicinal Products

Table 3 provides the details of the investigational medicinal products (IMPs).

TABLE 3
Summary of investigational medicinal products

| | | | | Route and | |
|---|---|---|---|---|---|
| | | | Dose of drug per | method of | |
| | Name of IMP | Pharmaceutical forms | administration | administration | Treatment duration |
| Double blind | Sarilumab or | Single-use 1.14 mL prefilled | Sarilumab 200 mg, | Subcutaneous | 52 weeks |
| phase of | matching placebo | glass syringes containing | or matching placebo | route | |
| sarilumab | | 175 mg/mL (200 mg) of | | | |
| | | sarilumab or placebo solution for | | | |
| | | injection | | | |
| Double blind | Sarilumab or | Single-use 1.14 mL prefilled | Sarilumab 150 mg | Subcutaneous | In the case of dose |
| phase of | matching placebo | glass syringes containing | (an option only for | route | reduction |
| sarilumab | | 131.6 mg/ml (150 mg) of | patients who had lab | | |
| | | sarilumab solution for injection | abnormality) or | | |
| | | | matching placebo | | |
| Double blind | Prednisone or | 1 and/or 5 mg over-encapsulated | | Oral | 52 weeks |
| phase | matching placebo | tablet or over-encapsulated | | administration | (combination of |
| | | placebo | | | prednisone/matching |
| | | | | | placebo*) |
| Open label ≤5 | Prednisone | 1 mg Commercial TABLETS | | Oral | In the case of flare |
| mg add-on | | | | administration | within the first 12 |
| | | | | | weeks after |
| | | | | | randomization |

*Sarilumab 200 mg Q2W + 14 Week GC taper arm: W 0/W 1: 15 mg; W 2: 14 mg; W 3: 12 mg; W 4: 10 mg; W 5: 9 mg; W 6: 8 mg; W 7: 7 mg; W 8: 6 mg; W 9: 5 mg; W 10: 4 mg; W 11: 3 mg; W 12: 2 mg; W 13: 1 mg; W 14 through W 51: matching placebo.
Placebo + 52 Week GC taper arm: W 0/W 1: 15 mg; W 2: 14 mg; W 3 through W 5: 12 mg; W 6/W 7: 10 mg; W 8 through W 11: 9 mg; W 12 through W 15: 8 mg; W 16 through W 19: 7 mg; W 20 through W 23: 6 mg; W 24 through W 27: 5 mg; W 28 through W 31: 4 mg; W 32 through 35: 3 mg; W 36 through 43: 2 mg; W 44 through W 51: 1 mg.

### Formulation

Sarilumab drug product was provided as single-use 1.14 ml prefilled glass syringes containing 131.6 mg/mL (150 mg), 175 mg/mL (200 mg) of sarilumab or placebo solution for SC injection. No preparation at the clinical site was required.

### Route of Administration

Sarilumab was administered subcutaneously (SC) in the abdomen or thigh when self-injections or also in upper arm (lateral side) by a professional or a non-professional caregiver. It was preferred that SC injection sites be alternated between the 4 quadrants of the abdomen (except the navel or waist area) or the thigh (front and side). Each drug administration required a single injection.

Patients and/or their non-professional caregivers were trained to prepare and administer study drug at the start of the study. This training was documented in the subject's study file. The study staff reviewed the patient's self-administration technique at visit 2 (week 0). For doses not given at the study site, diaries were provided to record information pertaining to those injections.

### Dose Regimen

The IMP (sarilumab or matching placebo) was administered every 14 days as per protocol IMP administration schedule; however, an IMP administration time window of ±3 days was permitted in exceptional circumstances (e.g., laboratory test result pending, or an ongoing AE or patient schedule difficulty). For subsequent IMP administrations the initial IMP administration schedule was followed again.

An interval of at least 11 days between 2 IMP (sarilumab and matching placebo) doses was maintained.

If the study visit was not performed at the site as scheduled, the dose was administered as described above, either by the patient, qualified site personnel, and/or their caregiver(s).

On the days when the patient has a study visit, the IMP was administered following clinic procedures and blood collection.

Patients were monitored for at least 30 minutes after each dose of sarilumab (or up to 2 hours as per country specific requirements) for any signs or symptoms of any medical events. In the case that the injection is administered by caregiver or self-injection, patients were instructed to monitor themselves for any signs or symptoms of any medical events. The total duration of treatment was 52 weeks.

### Dose Modification/Reduction

Sarilumab dose could be reduced to 150 mg q2w in a blinded manner to treat neutropenia, thrombocytopenia and/or elevated liver transaminases. In addition, sarilumab dose could be temporarily discontinued. The decision to reduce the sarilumab dose and/or to temporarily discontinue was made by the investigator.

Between the protocol-scheduled on-site visits, interim visits may have been required for IMP dispensing. As an alternative to these visits, sarilumab or matching placebo could be supplied from the site to the patient via a Sponsor-approved courier company where allowed by local regulations and approved by the subject.

Post-trial access to sarilumab was in compliance with all applicable national and local laws and regulations, including safety reporting obligations.

### Prednisone or Matching Placebo

### Formulation

1 and/or 5 mg over-encapsulated tablet or over-encapsulated matching placebo.

### Route of Administration

Prednisone or matching placebo was administered orally.

### Dose Regimen

All patients received prednisone treatment with a different regimen depending on the assigned group. Patients received prednisone and/or prednisone matching placebo in order to ensure the double-blind CS tapering regimen as defined below was maintained. The initial dose of prednisone for both groups was 15 mg/day for the first 2 weeks after randomization.

For Group 1: From week 2 to week 13, patients received gradually decreasing dose levels of prednisone. From week 14 onwards, patients without flare received prednisone matching placebo.

For Group 2: From week 2 to week 51, patients will received gradually decreasing dose levels of prednisone (prednisone or combination of prednisone and placebo to prednisone).

There were 2 phases to each double-blind prednisone taper regimen: phase 1: Initial 2 weeks after randomization (beginning of Week 0 to end of Week 1) when all patients received prednisone 15 mg/day and phase 2: 50 weeks (beginning of Week 2 to end of Week 51) following Phase 1. Prednisone and matching placebo were used to maintain the blinding of the tapering regimen for both treatment arms.

During the prednisone taper, patients were supplied with a monthly kit containing 4 weekly blister packs which clearly indicated the number of tablets to be taken per day. The monthly kits and blister packs were numbered, and it was important that the blister packs were used in sequential order and that 7 days of prednisone from each blister be taken by the patient before starting the next blister pack. If a scheduled study visit occurred before the target date, the patient completed the blister pack assigned for that particular week before using the sequentially numbered blister packs dispensed at the scheduled study visit. If tablets were missed or skipped, patients should not have used the missed tablets before resuming on the blister packs as scheduled.

Patients were trained in the use of the prednisone treatment kit and weekly prednisone blister packs at the baseline visit. This training included instruction on use of the blister packs in the appropriate order and use of the “spare” row of tablets in the case of loss of a tablet during dosing.

The daily encapsulated dose contained prednisone, placebo, or a combination of the two. The number of tablets to be taken each day varied but was not consistent with the dosage of prednisone. The number of tablets to be taken daily could increase or decrease during the tapering schedule but would not exceed 6 tablets per day.

Add-on Prednisone Taper in the Case of Flare within Initial 12 Weeks

During the initial 12 weeks of prednisone taper, treatment for one flare before week 12 was permitted if it could be successfully treated with a low dose of prednisone (≤5 mg/day) to add on to the CS tapering regimen provided that all other sustained remission parameters were met. The add-on prednisone must have been completed by week 12.

The prednisone for this add-on taper was provided in an open label manner consisting of count blister (1 mg each) in a child resistant wallet. It was up to the investigator to decide on the CS dosage (with a maximum dose of (≤5 mg/day) and taper schedule/duration according to the patient's disease status. The quantity of tablets to be taken daily was written on the wallet and recorded in the eCRF. The patient needed to be monitored and in close communication with the site to ensure patients took the correct quantity of prednisone until the add-on taper was completed prior to week 12.

### Non-Investigational Medicinal Products

Prednisone for rescue therapy: if a patient experienced a disease flare or could not adhere to the per protocol prednisone tapering schedule including the 5 mg prednisone add-on prior to Week 12, then the patient had to stop the per protocol prednisone taper and instead could receive commercial CS as a form of rescue therapy, per investigator's clinical judgment. The commercial CS was reimbursed by the Sponsor. The patient could continue in the double-blind period of the study for the full 52 weeks and could continue to receive blinded sarilumab or matching placebo injections unless contraindicated by safety concerns and complete the remainder of the study assessments. Once the patients were on the rescue therapy at the discretion of investigators, it was not allowed for them to return to per protocol prednisone taper regimen.

### Concomitant Medication

A concomitant medication is any treatment received by the patient concomitantly with any IMP(s).

The use of any biologics for treatment of PMR during the study was not permitted throughout the study treatment and until 6 weeks following the last sarilumab or matching placebo administration unless otherwise indicated. If any of these treatments were used, the patient was discontinued from IMP treatment but the patient remained in the study and continued to be monitored for safety.

Administration of any live (attenuated) vaccine was contraindicated until 3 months following the last sarilumab or matching placebo administration.

Treatment with non-biological disease modifying anti-rheumatic drugs [DMARDs] (such as alkylating agents, hydroxychloroquine, CsA, MMF, AZA, etc). was not permitted during the course of the study, unless used for the purpose of rescue therapy.

During the course of study, for patients in need of rescue therapy as per investigator judgment, corticosteroids were the agent of first choice. Patients could continue SC administration of sarilumab or matching placebo only if CS was used as rescue therapy. If the patients remained symptomatic despite CS rescue therapy, then other treatment options including non-biological immunosuppressive drugs could be used (patient must have had symptomatic PMR disease) and the patient was discontinued from the study treatment and considered a non-responder.

Methotrexate with a dose not exceeding 15 mg per week was permitted if the dose was stable for at least 3 months prior to baseline. The dose remained stable (could be reduced or discontinued for safety reasons, if necessary), throughout the study treatment duration and until 6 weeks following the last SC IMP (sarilumab or matching placebo) administration.

Treatment with any IMP other than sarilumab and CS defined by protocol was not permitted.

### Steroids

There were two standardized prednisone-tapering regimens in the study with one that lasted for 14 weeks (Group 1) and the other one that lasted for 52 weeks (Group 2). The total duration of prednisone therapy for each particular patient depended on the treatment group to which the patient was randomized.

If the patient developed an adverse event (AE) for a condition not related to PMR that required the introduction of a new systemic CS medication, the new medication and AE were recorded on the patient eCRF. Furthermore, the Sponsor was notified as soon as possible at the time of the steroid dose modification (e.g., within 24 hours) in order to discuss the patient's status with regards to ongoing study participation. Intranasal, inhaled, ophthalmic or topical CSs as per label were permitted throughout the course of the study.

### Nonsteroidal Anti-Inflammatory Drugs and Analgesics

As there were limited treatment options for pain, all analgesics, including nonsteroidal anti-inflammatory drugs (NSAIDs), were allowed. These analgesics were held for 24 hours prior to efficacy assessment, including physical function and quality of life assessments.

Acetaminophen use was limited to <4 g every 24 hours. Specific attention was paid to co-administration of hepatotoxic drugs.

### Treatment for Dyslipidemia

Treatments for dyslipidemia, such as statins, were permitted. Doses of medications for dyslipidemia were stable for at least 6 weeks prior to screening visit. Any change and reason for change were recorded on the patients' electronic case report form (eCRF). Anti-IL-6 drugs, including sarilumab are known to increase serum total cholesterol and this effect was closely monitored during the study. If, during the treatment period of this study, patients were found to have significant increase in cholesterol levels, or other lipid abnormalities, then cholesterol lowering therapy with statins, or other treatment(s) for dyslipidemia, per local guidelines, were initiated or the dose adjusted.

### Glucocorticoid-Induced Osteopenia/Osteoporosis Prevention and Treatment

Oral calcium, 25-hydroxy vitamin D supplementation, and bisphosphonate therapy (e.g., alendronate 70 mg weekly or zoledronate 4 mg annually) for the prevention or treatment of glucocorticoid-induced osteoporosis were permitted. The doses and treatment duration complied with local practice or clinical guidelines at the discretion of the Investigator.

### CYP Substrates

IL-6 has been shown to reduce Cytochrome P450 (CYP)1A2, CYP2C9, CYP2C19, and CYP3A4 enzyme expression in in vitro studies. Therefore, it was expected that for a molecule that antagonizes cytokine activity, such as sarilumab, the formation of CYP450 enzymes could be normalized, and as a result drugs that are metabolized by these CYP450 isoforms could have had decreased levels when PMR patients start receiving sarilumab. As a precautionary measure, drugs which are metabolized via these cytochromes and with a narrow therapeutic index were adjusted if needed: doses were increased to maintain efficacy after initiation of sarilumab and decreased after sarilumab was stopped. Some examples of CYP450 substrates with a narrow therapeutic index, requiring monitoring of effect are warfarin or monitoring of drug concentration include, but are not limited to, the following: warfarin, cyclosporine, theophylline, digoxin, antiepileptics, such as carbamazepine (CARBATROL®, TEGRETOL®, divalproex (DEPAKOTE®), phenytoin (DILANTIN®), or valproic acid (DEPAKENE®); or antiarrhythmics, such as disopyramide (NORPACE®), procainamide (PROCAN®, PRONESTEC), or quinidine (QUINIDEX®, QUIN RELEASE QUIN-G®).

### Primary Endpoint

### Proportion of Patients Achieving Sustained Remission at Week 52

Sustained remission at week 52 is defined by having met all of the following parameters: achievement of disease remission no later than week 12 AND absence of disease flare from week 12 through week 52 AND sustained reduction of CRP (to <10 mg/L, with an absence of successive elevations to ≥10 mg/L) from week 12 through week 52 AND successful adherence to the prednisone taper from week 12 through week 52. Sustained remission was also assessed between week 16 through week 52 and from and week 24 to week 52.

Successful adherence to the prednisone taper may include the use of any excess prednisone (beyond the per protocol CS tapering regimen) with a cumulative dose of less than or equal to 100 mg (or equivalent), such as those employed to manage an AE not related to PMR.

Disease remission is defined as: resolution of signs and symptoms of PMR, and normalization of CRP (<10 mg/L). A single CRP elevation (≥10 mg/L) was not considered absence of remission unless CRP remained elevated (≥10 mg/L) for two consecutive study visits.

Flare is defined as: either 1) recurrence of signs and symptoms attributable to active PMR plus an increase in CS dose due to PMR, or 2) elevation of ESR attributable to active PMR plus an increase in CS dose due to PMR.

Increase in CS dose is defined as: Any dose increase during the protocol-defined steroid taper or re-initiation of prednisone therapy after the protocol defined taper has been completed. During the initial 12 weeks of prednisone taper, treatment for one flare before week 12 is permitted if it could be successfully treated with a low dose (<5 mg/day) prednisone add-on taper regimen (completed prior to week 12) and provided that all other sustained remission parameters were met.

Signs and symptoms of PMR: Evaluation for clinical signs and symptoms by the Efficacy Assessor at every study visit according to the schedule of assessment should include, but are not limited to, the following: morning stiffness and/or pain, in the neck, shoulder and/or hip girdles; limited range of motion of the shoulders and/or hip girdles; constitutional symptoms, such as fatigue, weight loss and low-grade fever; and other features judged by the clinician-investigator to be consistent with a PMR flare.

### Secondary Efficacy Endpoints

### Components of Sustained Remission Composite Measure at Week 52

patients who achieved disease remission by week 12, week 16 through week 52, and week 24 through week 52; patients who have absence of disease flare from week 12 through week 52; patients who have normalization of CRP (decrease to <10 mg/L) with sustained normalization from either week 12 through week 52, week 16 through week 52, and week 24 through week 52; and patients who successfully adhere to the prednisone taper from week 12 through week 52.

Successful adherence to the prednisone taper could include the use of any excess prednisone (beyond the per protocol CS tapering regimen) with a cumulative dose of less than or equal to 100 mg (or equivalent), such as those employed to manage an AE not related to PMR.

### Total Cumulative Corticosteroid (Including Prednisone) Dose Over 52 Weeks

The total cumulative prednisone (or equivalent) dose over the 52-week period for each group was analyzed as a secondary endpoint.

### Time to First PMR Flare

The duration to first PMR flare from clinical remission up to 52 weeks for each group was analyzed.

### Composite Glucocorticoid Toxicity Index and Components

Glucocorticoid toxicity index (GTI) is a composite scale designed to assess glucocorticoid related morbidity and potential steroid-sparing effect of treatment alternatives. The Composite GTI and Specific List constitute the overall GTI. The Composite GTI consists of nine domains and 31 items that assess the potential side effects of glucocorticoid, and include evaluation of body mass index (BMI), glucose tolerance, blood pressure, lipid metabolism, bone mineral density, glucocorticoid-induced myopathy, skin toxicity, neuropsychiatric toxicity and infection. These were the potential CS toxicities that were likely to occur during the course of a clinical trial and could vary depending on the extent of CS exposure, and that are weighted and scored.

The domains of the Composite GTI and the specific list of the GTI were assessed at baseline, week 12 (Visit 6), week 24 (Visit 9), week 40 and week 52 (except bone density which was assessed at Baseline and Week 52 only). Glucocorticoid (GC) toxicity or the changes in GC toxicity (comparison with baseline data) for each domain were scored (score range from −36 to 439) based on the information from laboratory, vital sign and clinical assessments and review of concomitant medications. The composite GTI was then reported as both a total score and domain-specific scores, in order to account for scenarios when improvements in certain domains compensate for worsening in others.

Bone mineral density assessment was performed at the baseline (visit 2) and week 52 (visit 12) using a Dual-Energy X-ray Absorptiometry (DXA) scan. The scan was performed within ±14 days of visit 2 and within −14 days of visit 12 and needed to include the lumbosacral and femoral neck regions. However, the baseline visit DXA scan was not required if there was one available within 12 weeks of baseline that included the assessment of the lumbosacral and femoral neck regions.

The Specific List consists of 11 domains and 23 items that are not weighted, and captures other CS related toxicities not found in the Composite GTI (see Table 4, below). Information related to the domains/items of the Specific List were collected if available at the scheduled time points (baseline, week 12, week 24, week 40 and week 52), but no pre-specified assessments related to the domains in the Specific List, unless for cause, were required within the conduct of this study.

TABLE 4
Specific list for glucocorticoid toxicity index

| | |
|---|---|
| At baseline | New since |
| or before | baseline |

Body mass index
An absolute increase in BMI of more than 8
units (and >24.9 kg/m²)
Blood pressure
Hypertensive emergency (see definition below)
PRES (posterior reversible encephalopathy
syndrome) (see definition below)
Endocrine
Symptomatic adrenal insufficiency
Bone health
Osteonecrosis of one joint
Osteonecrosis of more than one joint
Bone mineral density decrease >6%
Insufficiency fracture
Insufficiency fracture in more than one bone
Muscle & tendon
Severe glucocorticoid myopathy (see
definition below)
Tendon rupture
More than one tendon rupture
Eye
Central serous retinopathy
New-onset or worsened elevation of intra-
ocular pressure requiring treatment or
change in treatment
Posterior subcapsular cataracts (or history of
same)
Infection
Grade 4 infection (see definition below)
Grade 5 infection (death from infection)
Glucose tolerance
Diabetic nephropathy
Diabetic neuropathy
Diabetic retinopathy
Gastrointestinal tract
Gastrointestinal perforation (occurring in the
absence of regularnonsteroidal anti-
inflammatory drug use)
Peptic ulcer disease confirmed by endoscopy
(excluding H. pylori)
Skin
Severe skin toxicity (see definition below)
Neuropsychiatric
Psychosis, defined as hallucinations, delusions,
or disorganized thought processes (occurring in
the absence of mania, delirium, or depression)
Glucocorticoid-induced violence toward self or
others
Other glucocorticoid toxicities
Please specify: _{——}_{——}_{——}
DEFINITIONS
Hypertensive emergency: the blood pressure has reached levels that are damaging organs. Hypertensive emergencies generally occur at blood pressure levels exceeding 180 mmHg systolic OR 120 mmHg diastolic, but can occur at even lower levels in patients whose blood pressure have not been elevated before. Complications can include: stroke, loss of consciousness, memory loss. myocardial infarction, hypertensive retinopathy or nephropathy, aortic dissection, angina, pulmonary edema.
Posterior reversible leukoencephalopathy syndrome (PRES): a clinical radiological entity. Clinical features may include headaches, altered mental status, seizures, and visual loss, depending on the affected neuroanatomy. Characteristic Magnetic Resonance Imaging (MRI) findings include vasogenic edema involving the white matter that predominantly affects the posterior occipital and parietal lobes of the brain, although other brain regions may also be affected. Confirmation by MRI is required as is exclusion of other potential causes (including hypertensive emergency).
Severe glucocorticoid myopathy: grade 3 or worse myopathic weakness or respiratory myopathic weakness attributable to glucocorticoid myopathy.
Central serous retinopathy: a fluid detachment of macula layers from their supporting tissue. Requires formal ophthalmology examination, typically accompanied by optical coherence tomography and/or fluorescein angiography for diagnostic confirmation.
Grade 4 infection: Life-threatening consequences (e.g., septic shock, hypotension. acidosis, necrosis).
Diabetic nephropathy: macroalbuminuria; i.e., a urinary albumin excretion >300 mg in a 24-hour collection or a urinary protein: creatinine ratio >300 mg/g.
Diabetic neuropathy: Any of four types of peripheral neuropathy occurring in the setting of diabetes mellitus, namely: 1) a distal sensory polyneuropathy; 2) autonomic neuropathy (hypoglycemia unawareness, bladder or bowel problems, erectile dysfunction, and other autonomic nervous system issues); 3) diabetic amyotrophy (muscle infarction); or 4) mononeuritis (e.g., foot drop attributed to diabetic neuropathy).
Diabetic retinopathy: any form of retinopathy associated with diabetes mellitus, including both non-proliferative and proliferative forms of diabetic retinopathy as well as diabetic macular edema. These complications must be confirmed by an ophthalmologist.
Severe skin toxicity: any of the three following manifestations:
Grade 4 acneiform lesions - papules and/or pustules covering any % body surface area (BSA), which may or may not be associated with symptoms of pruritus or tenderness and are associated with extensive superinfection with IV antibiotics indicated or life-threatening consequences
Grade 3 striae - covering >30% BSA or associated with ulceration
Grade 3 ulcers - combined area of ulcers >2 cm or full-thickness skin loss involving damage to or necrosis of subcutaneous tissue that may extend down to fascia

### Other Endpoints

GTI aggregate improvement score (AIS) at week 52.

GTI cumulative worsening score (CWS) at week 52.

Secondary safety endpoints: adverse events (AE), laboratory safety variables, vital signs variables, antinuclear antibodies, and -immunogenicity variables.

Functional sarilumab concentrations in serum.

Physician global assessment of disease activity as measured by MD-VAS.

Patient-reported outcomes (described in the section below).

Changes in ESR and CRP from baseline through week 52.

Changes in IL-6 Level and Soluble IL-6 Receptor (sIL 6R) Through Week 52.

Changes in markers of inflammation and disease activity over time as assessed in circulating immune cell types, circulating proteins, and gene expression changes.

### Patient-Reported Outcomes

Patients were asked to complete the patient-reported outcome (PRO) questionnaires described below at visit 2 (baseline visit), visit 6 (week 12), at visit 9 (week 24), and visit 12 (week 52). The following metrics were assessed.

### The Functional Assessment of Chronic Illness Therapy Fatigue Scale

As shown in Table 5 below, the functional assessment of chronic illness therapy fatigue scale (FACIT-Fatigue) is a generic PRO instrument which includes 13 items to measure fatigue. Each item is rated by patients on a 0 to 4 scale (0=not at all, 1=a little bit, 2=somewhat, 3=quite a bit, 4=very much). Scores are summarized to give a total score between 0 and 52. The recall period was the last 7 days.

TABLE 5
FACIT-Fatigue scale

| | Not at | A little | Some- | Quite | Very |
|---|---|---|---|---|---|
| | all | bit | what | a bit | much |
| I feel fatigued | 0 | 1 | 2 | 3 | 4 |
| I feel weak all over | 0 | 1 | 2 | 3 | 4 |
| I feel listless (“washed out”) | 0 | 1 | 2 | 3 | 4 |
| I feel tired | 0 | 1 | 2 | 3 | 4 |
| I have trouble starting things | 0 | 1 | 2 | 3 | 4 |
| because I am tired | | | | | |
| I have trouble finishing | 0 | 1 | 2 | 3 | 4 |
| things because I am tired | | | | | |
| I have energy | 0 | 1 | 2 | 3 | 4 |
| I am able to do my usual | 0 | 1 | 2 | 3 | 4 |
| activities | | | | | |
| I need to sleep during the day | 0 | 1 | 2 | 3 | 4 |
| I am too tired to eat | 0 | 1 | 2 | 3 | 4 |
| I need help doing my usual | 0 | 1 | 2 | 3 | 4 |
| activities | | | | | |
| I am frustrated by being too | 0 | 1 | 2 | 3 | 4 |
| tired to do the things I want | | | | | |
| to do | | | | | |
| I have to limit my social | 0 | 1 | 2 | 3 | 4 |
| activity because I am tired | | | | | |

### EQ-5D-3L

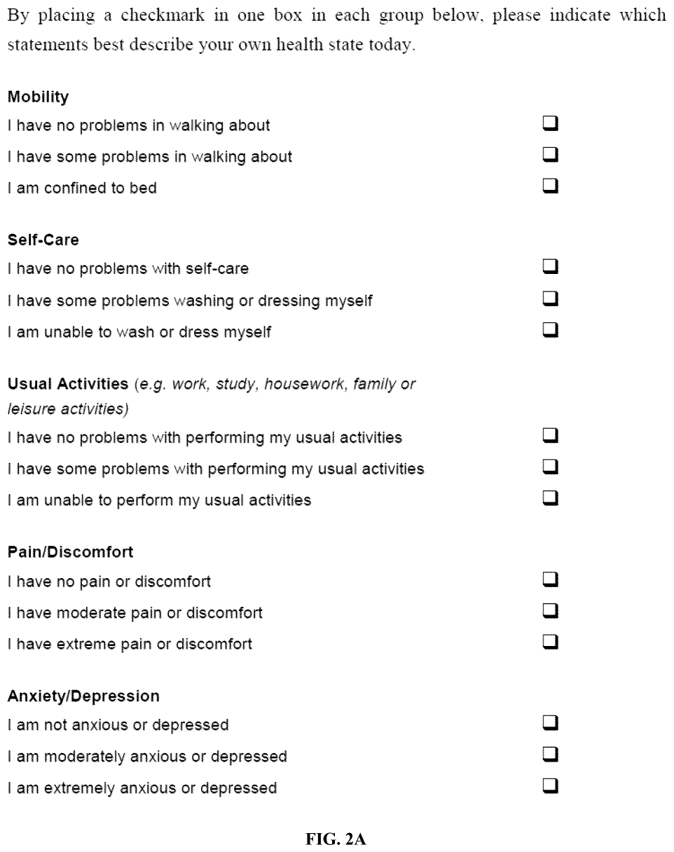

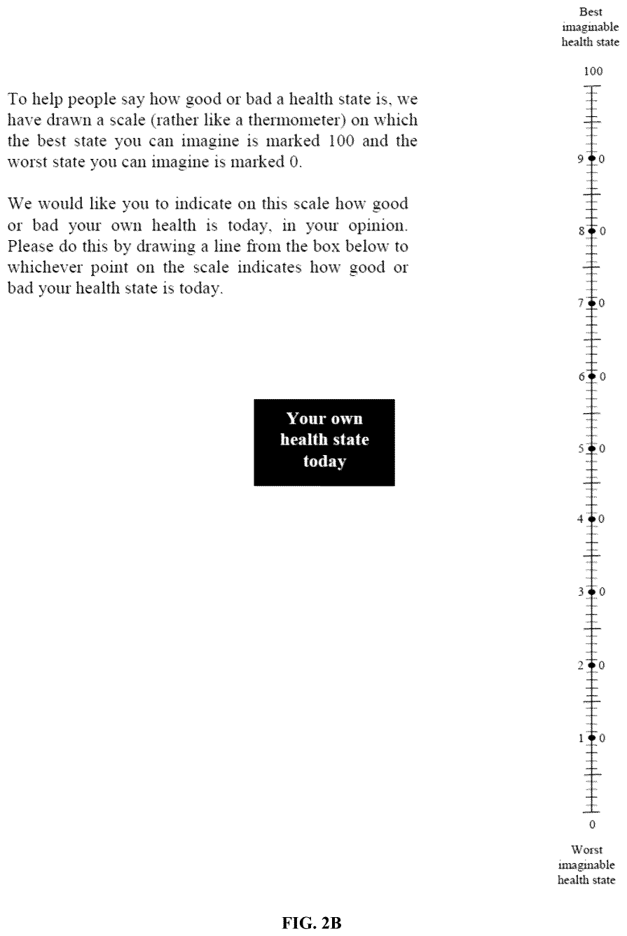

As shown in FIG. 2A-B, the EQ-5D-3L is a generic PRO instrument which measures health status (EuroQol Group, EuroQol-a new facility for the measurement of health-related quality of life, Health Policy 1990; 16(3):199-208).

There are two components to the EQ-5D; a health utility index score derived from 5 items addressing mobility, self-care, usual activities, pain/discomfort, and anxiety/depression “today”, and a current (“right now”) general health status score derived from a single 0-100 Visual Analog Scale (VAS). The items contributing to the EQ-5D-3L health utility index score each have the same 3-point response scale (1=no problem, 2=moderate problems, 3=severe problems). The VAS is anchored with ‘best imaginable health state’ and ‘worst imaginable health state’.

### Short Form 36v2 (SF-36v2)

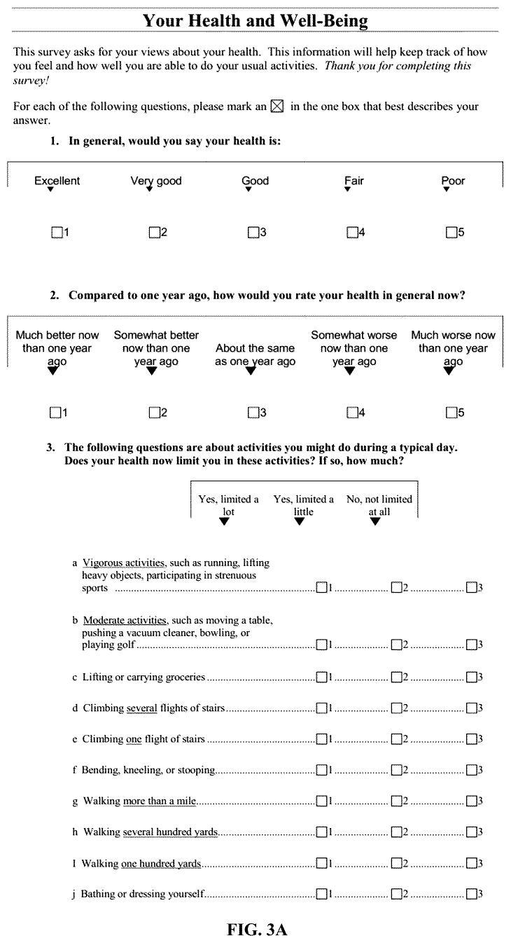

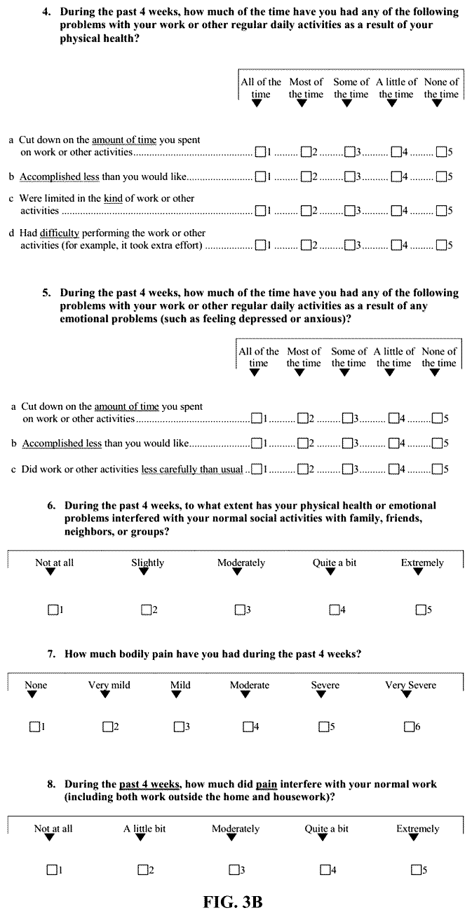

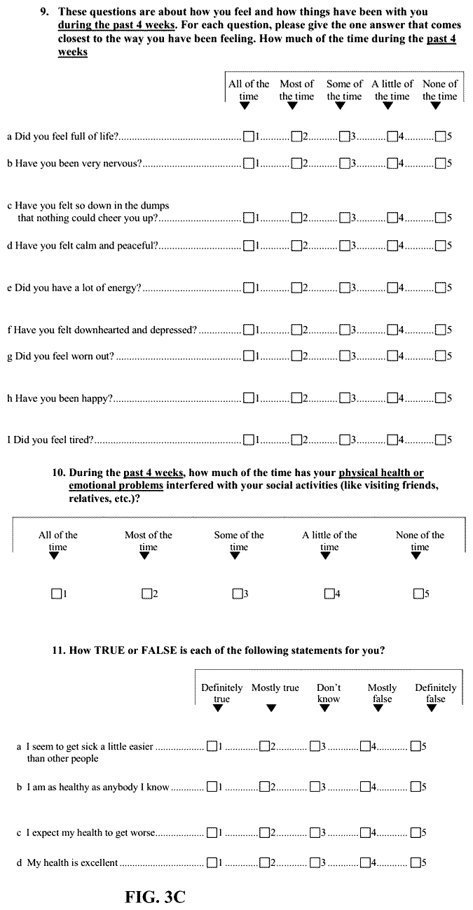

As shown in FIG. 3A-C, the Short Form 36v2 (SF-36v2) is a short-form generic, 36-item PRO instrument that evaluates 8 multi-item dimensions of health: physical functioning (PF; 10 items), social functioning (SF; 2 items), role limitations due to physical problems (RP; 4 items), role limitations due to emotional problems (RE; 3 items), mental health (MH; 5 items), energy/vitality (VT; 4 items), bodily pain (BP; 2 items), and general health perception (GH; 5 items) (Ware, et al. The MOS 36-Item Short-Form Health Survey (SF-36): I. Conceptual Framework and Item Selection, Medical Care 1992; 30(6):473-483). For each dimension, item scores are coded, summed, and transformed on to a scale from 0 (worst possible health state measured by the questionnaire) to 100 (best possible health state). Two standardized summary scores can also be calculated from the SF-36v2; the physical component summary (PCS) and the mental health component summary (MCS) on a scale from 0-100 (See Maruish ME (2011) User's manual for the SF-36v2 Health Survey (3rd ed).. Lincoln, RI: QualityMetric Incorporated).

### HAQ-DI

The HAQ-DI was developed to assess physical functional status in adults with arthritis but is now commonly used among many rheumatologic conditions (See Wolfe F “A brief clinical health assessment instrument: CLINHAQ” Arthritis Rheum. 1989; 32 (suppl): S9 and Wolfe F. “Data collection and utilization: a methodology for clinical practice and clinical research” Rheumatoid arthritis: pathogenesis, assessment, outcome and treatment, New York: Marcel Dekker, 1994: 463-514). It contains 25 items: 20 4-point Likert-scale questions assessing 8 physical dimensions of activities of daily living (dressing and grooming, arising, eating, walking, hygiene, reaching, gripping, and errands and chores), 13 additional questions assessing use of assistive devices, and 8 additional questions assessing help received from another. The recall period is the last week. To calculate the HAQ-DI Score, there are 3 steps: sum the 8 category scores by using the highest sub-category score from each category; adjust for use of aids/devices and/or help from another person when indicated; and divide the summed category scores by the number of categories answered (must be a minimum of 6) to obtain a HAQ-DI score of 0-3 (3=worst functioning).

In addition, the HAQ-DI has two additional questions, measured on 0-100 scales: How much pain have you had in the past week? Please rate how well you are doing on a scale of 0 to 100 (0 represents “very well” and 100 represents “very poor” health). These questions, measuring pain and global assessment respectively, are independently scored.

### Clinician-Reported Outcomes

### Physician Global Assessment of Disease Activity-Visual Analog Scale [MD-VAS]

As shown in FIG. 4, the efficacy assessor was requested to rate the patient's disease activity on an anchored 100 mm horizontal VAS where 0 is considered not active and 100 is considered the most active (See Huskisson et al. “Vertical or Horizontal Visual Analogue Scales” Ann Rheum Dis. 1979 December; 38(6):560).

### Other Assessments

### PMR Activity Score (PMR-AS)

PMR-AS is calculated as the sum of CRP (mg/dL), visual analog score (VAS) for pain (0 to 10), VAS for physician's assessment (0 to 10), duration of morning stiffness (MST [min] X and the ability to elevate the upper limbs (EUL [3-0]).

### Glucocorticoid Toxicity Index

Glucocorticoid Toxicity Index (GTI) is a composite scale designed to assess glucocorticoid related morbidity. GTI-cumulative worsening score (CWS) captures cumulative glucocorticoid toxicity regardless of whether it is permanent or transient. The GTI-CWS can only increase or remain the same over time. A lower score indicates lower glucocorticoid toxicity. The GTI-aggregate improvement score (AIS) captures both worsening and improvement in glucocorticoid toxicity. New or worsening toxicities contribute a positive score and improvements in existing toxicities contribute a negative score. A lower score indicates lower glucocorticoid toxicity.

### Cumulative Corticosteroid Dose

The cumulative corticosteroid dose is a measure of a patient's exposure to corticosteroids over a period of time, such as 14 or 52 weeks.

### Pharmacodynamics, Pharmacokinetics and Anti-Drug Antibodies

### Pharmacodynamic Variables Determined

Changes in ESR and CRP from baseline through week 52.

Changes in IL-6 level and soluble IL-6 receptor (sIL-6R) through week 52.

Changes in markers of inflammation and disease activity over time as assessed in circulating immune cell types, circulating proteins, and gene expression changes as follows: Markers of inflammation were assessed in a subset of patient population during the study at V2 (baseline), V4 (week 4), V9 (week 24) as measured by immune cell phenotyping. Approximately patients (40 patients from each treatment arm) were selected for this analysis. Disease activity assessment of PMR patients was assessed via evaluation of circulating proteins and gene expression. Serum or plasma samples were collected at V2 (baseline), V3 (week 2), V6 (week 12), V9 (week 24), V12 (week 52 EOT) for circulating protein measurements. DNA samples were collected at V2 (baseline), or any treatment or follow up visit, RNA samples will be collected at V2 (baseline) and/or pre-dose at V3 (week 2) for gene expression measurement.

### Pharmacokinetics and Anti-Drug Antibodies

Pre-dose blood samples at each study visit were collected for determination of serum sarilumab concentration (functional), and antibodies to sarilumab and analyzed according to the bioanalytical methods shown in Table 6. Pre-dose serum sarilumab concentrations at week 0 and sarilumab trough levels at weeks 2, 4, 12, 16, 24, 32, 52 and week 58 were collected. In addition, post-dose samples were taken 4-7 days after the week 24 (Visit 9).

TABLE 6
Summary of bioanalytical methods for functional
sarilumab and anti-sarilumab antibody

| Bioanalysis | Functional sarilumab | Anti-sarilumab antibody |
|---|---|---|
| Matrix | Serum | Serum |
| Analytical technique | ELISA | Electrochemiluminescence |
| Site of bioanalysis | Regeneron | Regeneron |

ELISA = enzyme-linked immunosorbent assay

### Adverse Events

An adverse event (AE) was defined as any untoward medical occurrence in a patient or clinical investigation patient administered a pharmaceutical product and did not necessarily have to have a causal relationship with this treatment. Reconciliation of AEs between the GTI and clinical safety database was part of the usual data review surveillance activities. All AEs, regardless of seriousness or relationship to IMP, spanning from the signature of the informed consent form until the end of the study, were recorded.

### Results

### Demographic and Other Baseline Characteristics

Demographic and participant characteristics at baseline were generally similar between treatment groups with a number of participants slightly higher in the age group of ≥75 to <85 years and with a slightly lower number of participants in the age group ≥65 to <75 years in the placebo +52-week taper group compared to the sarilumab 200 mg q2w+14-week taper group.

Baseline disease characteristics were generally well balanced between the treatment groups except for the number of participants with other joint involvement which was more frequently reported in the sarilumab 200 mg q2w+14-week taper group compared to the placebo +52-week taper group (12 [20.0%] versus 6 [10.3%]). The mean (SD) duration of PMR for all participants was 631.3 (752.0) days. Participants in placebo+52-week taper group had a longer duration of morning stiffness as measured by minutes; however, the number of participants with duration of morning stiffness >45 minutes were comparable between treatment groups. A detailed breakdown of patient demographics and baseline disease characteristics is provided in Table 7, below.

TABLE 7
Patient demographics and characteristics

| | Sarilumab + 14-week | | Placebo + 52-week | |
|---|---|---|---|---|
| Parameter | GC taper (n = 60) | | GC taper (n = 58) | |
| Median age, years | 69 | (51-88) | 70 | (52-88) |
| ≥50 to <65 years, n (%) | 16 | (26.7) | 15 | (25.9) |
| ≥65 to <75 years, n (%) | 34 | (56.7) | 26 | (44.8) |
| ≥75 to <85 years, n (%) | 9 | (15.0) | 16 | (27.6) |
| ≥85 years, n (%) | 1 | (1.7) | 1 | (1.7) |
| Sex (female), n (%) | 45 | (75.0) | 37 | (63.8) |
| Race, n (%) | | | | |
| Caucasian | 50 | (83.3) | 48 | (82.8) |
| Asian | 1 | (1.7) | 2 | (3.4) |
| Not reported | 9 | (15.0) | 8 | (13.8) |
| Median PMR duration (days)* | 292 | (78-3992) | 310 | (66-2784) |
| Number of prior flares/patient, median | 2 | (1-14) | 2 | (1-22) |
| (range) | | | | |
| Prednisone or equivalent GC dose for PMR | 11.25 | (7.5; 20.0) | 10.00 | (7.5; 20.0) |
| (mg/day), median (range) | | | | |
| Prior immunosuppression, n (%) | | | | |
| Methotrexate | 5 | (8.3) | 10 | (17.2) |
| Leflunomide | 2 | (3.3) | 1 | (1.7) |
| Azathioprine | 0 | | 1 | (1.7) |
| Hydroxychloroquine | 1 | (1.7) | 1 | (1.7) |
| Adalimumab | 1 | (1.7) | 0 | |
| Tocilizumab | 0 | | 1 | (1.7) |
| Median CRP, mg/L | 6.8 | (0.5-38.2) | 5.7 | (0.1-62.3) |
| Median ESR, mm/h | 25.0 | (2.0-115.0) | 22.0 | (5.0-85.0) |

*Diagnosis date to baseline. Placebo + 52-week GC taper n = 50, Sarilumab 200 mg Q2W + 14-week GC taper n = 54. CRP, C-reactive protein; ESR, erythrocyte sedimentation rate; GC, glucocorticoid; PMR, polymyalgia rheumatica.

### Exposure

The cumulative exposure to double-blind treatment and median duration of study treatment were similar between the sarilumab 200 mg q2w+14-week taper group and placebo+52-week taper group. The cumulative exposure to treatment was 47.37 patient years for the sarilumab 200 mg q2w+14-week taper group and 45.36 patient years for the placebo+52-week taper group.

### Overall Summary of Efficacy Endpoints

An overall summary of the efficacy endpoints is provided in Table 8. The results of this study demonstrated that treatment of participants with PMR with sarilumab 200 mg q2w+14-week taper provided greater benefit than treatment with a 52-week CS taper alone. The study met its primary endpoint as the proportion of participants achieving sustained remission at Week 52 was higher in the sarilumab 200 mg q2w+14-week taper group compared to the placebo +52-week taper group; and this difference was statistically significant. The study also showed that a higher proportion of patients in the sarilumab arm, compared with the comparator arm, achieved sustained remission when assessed from week 12, week 16, and week 24 up to week 52. Most patients who achieved sustained remission did so rapidly by week 12 with some additional responses seen between week 16 and 24.

The results of the sensitivity analyses excluding CRP for the primary efficacy endpoint were consistent with those from the primary analysis. The majority of secondary endpoints were al so achieved.

TABLE 8
Summary of efficacy endpoints - Intent-to-Treat (ITT) Population

| | | |
|---|---|---|
| Placebo + | Sarilumab 200 mg | |
| 52-week taper | q2w + 14-week taper | P-value vs |
| (n = 58) | (n = 60) | Placebo |

Endpoints included in the hierarchical testing procedure

Primary endpoint
Sustained remission at Week 52^{a}

| | | | | | |
|---|---|---|---|---|---|
| Number (%) of patients with sustained | 6 | (10.3) | 17 | (28.3) | |
| remission at Week 52 | | | | | |
| Proportion difference (95% CI) vs placebo | | | 18.0 | (4.15, 31.82) | 0.0193^{b} |
| Sustained remission at Week 52^{a}, excluding | | | | | |
| acute phase reactants | | | | | |
| Sensitivity analysis | | | | | |
| Number (%) of patients with sustained | 8 | (13.8) | 19 | (31.7) | 0.0280^{b} |
| remission at Week 52 | | | | | |
| Proportion difference (95% CI) vs placebo | | | 17.9 | (3.13, 32.61) | |
| Secondary endpoint | | | | | |
| Cumulative corticosteroid dose during | | | | | |
| 52-week treatment period | | | | | |
| Medan (95% CI) for expected cumulative | 2044.0 | (2031.0, 2044.0) | 777.0 | (777.0, 777.0) | NA |
| CS dose in mg^{c} | | | | | |
| Medan (95% CI) for actual cumulative CS | 2094.0 | (2044.0, 2455.0) | 777.0 | (777.0, 777.0) | <.0001^{e} |
| dose in mg^{d} | | | | | |
| Medan of difference (95% CI) between | 199.5 | (0.0, 496.0) | 0.0 | (0.0. 0.0) | 0.0189^{e} |
| actual and expected cumulative CS dose | | | | | |
| in mg | | | | | |
| Other secondary/exploratory endpoints | | | | | |
| Components of sustained remission at | | | | | |
| Week 52, n (%) | | | | | |
| Achievement of disease remission no later | 22 | (37.9) | 28 | (46.7) | |
| than Week 12^{f} | | | | | |
| Absence of disease flare from Week 12 | 19 | (32.8) | 33 | (55.0) | |
| through Week 52^{g} | | | | | |
| Sustained reduction of CRP from Week 12 | 26 | (44.8) | 40 | (66.7) | |
| through Week 52^{h} | | | | | |
| Successful adherence to the prednisone | 14 | (24.1) | 30 | (50.0) | |
| taper from Week 12 through Week 52ⁱ | | | | | |
| Time to first PMR flare after clinical | | | | | |
| remission up to 52 weeks^{j} | | | | | |
| Number (%) of patients with PMR flare | 17 | (29.3) | 10 | (16.7) | |
| after clinical remission | | | | | |
| Kaplan-Meier estimates in days, median | 99.00 | (1.000; 154.000) | NC | (93.000: NC) | |
| (95% CI)^{k} | | | | | |
| Hazard ratio (95% CI)^{l} | | | 0.56 | (0.35, 0.90) | 0.0153 |
| Glucocorticoid toxicity index (GTI) scores | | | | | |
| Cumulative worsening score (CWS) at | | | | | |
| Week 52 | | | | | |
| LS mean (SE)ⁿ | 57.22 | (6.678) | 52.32 | (6.507) | |
| LS mean difference vs placebo (95% CI)^{m} | | | −4.90 | (−23.477, 13.669) | 0.6020 |
| Aggregate improvement score (AIS) at | | | | | |
| Week 52 | | | | | |
| LS mean (SE)ⁿ | 2.57 | (6.275) | −4.02 | (6.115) | |
| LS mean difference vs placebo (95% CI)^{m} | | | 6.59 | (−24.047, 10.862) | 0.4559 |
| PMR activity score (PMR-AS) at Week 52 | | | | | |
| LS mean change from baseline (SE)ⁿ | −10.27 | (0.97) | −15.57 | (0.94) | 0.0002 |
| LS mean difference for change from | | | −5.302 | (−8.006, −2.597) | |
| baseline vs placebo (95% CI)ⁿ | | | | | |
| Quality of life endpoints | | | | | |
| EQ-5D - Single index utility at Week 52 | | | | | |
| LS mean change from baseline (SE)ⁿ | −0.02 | (0.04) | 0.11 | (0.04) | |
| LS mean difference for change from | | | 0.130 | (0.010, 0.250) | 0.0336 |
| baseline vs placebo (95% CI)ⁿ | | | | | |
| SF-36 physical component summary score at | | | | | |
| Week 52 | | | | | |
| LS mean change from baseline (SE)ⁿ | 2.87 | (1.45) | 7.65 | (1.34) | |
| LS mean difference for change from | | | 4.784 | (0.865, 8.703) | 0.0172 |
| baseline vs placebo (95% CI)ⁿ | | | | | |
| SF-36 mental component summary score at | | | | | |
| Week 52 | | | | | |
| LS mean change from baseline (SE)ⁿ | −1.71 | (1.57) | 3.04 | (1.47) | |
| LS mean difference for change from | | | 4.748 | (0.484, 9.013) | 0.0295 |
| baseline vs placebo (95% CI)ⁿ | | | | | |
| MD-VAS at Week 52 | | | | | |
| LS mean change from baseline (SE)ⁿ | −30.49 | (3.48) | −40.58 | (3.40) | |
| LS mean difference for change from | | | −10.097 | (−19.775, −0.419) | 0.0411 |
| baseline vs placebo (95% CI)ⁿ | | | | | |
| FACIT-Fatigue at Week 52 (0-52) | | | | | |
| LS mean change from baseline (SE)ⁿ | 4.17 | (1.42) | 7.91 | (1.33) | |
| LS mean difference for change from | | | 3.740 | (−0.108, 7.588) | 0.0567 |
| baseline vs placebo (95% CI)ⁿ | | | | | |
| HAQ-DI standardized score at Week 52 | | | | | |
| LS mean change from baseline (SE)ⁿ | −0.15 | (0.09) | −0.39 | (0.09) | |
| LS mean difference for change from | | | −0.246 | (−0.496, 0.005) | 0.0543 |

baseline vs placebo (95% CI)ⁿ

Abbreviations: ANCOVA, analysis of covariance; AIS, aggregate improvement score; CI, confidence interval; CRP, C-reactive protein; CS, corticosteroids; CWS, cumulative worsening score; EQ-5D-3L, EuroQol 5 dimension questionnaire 3-level version; ESR, erythrocyte sedimentation rate; FACIT, functional assessment of chronic illness therapy; GTI, glucocorticoid toxicity index; HAQ-DI, health assessment questionnaire disability index; ITT, intent-to-treat; LS, least squares; MMRM, mixed-effect model repeated measure; NC, not able to calculate; NA, not applicable; PMR, polymyalgia rheumatica; PMR-AS, PMR activity score; q2w, every 2 weeks; SE, standard error; SF-36, short-form 36-item questionnaire; VAS, visual analogue scale.
^{a}Patients who did not achieve remission, received rescue treatment with open label prednisone (or corticosteroid dose equivalents), withdrew from the study before Week 52, or had missing data that prevented assessment of the primary endpoint were considered as non-responders.
^{b}p-value from Fisher's exact test.
^{c}Expected cumulative dose based on the CS tapering regimen up to the end of treatment, assuming that the taper was continued without error.
^{d}Cumulative dose of CS up to the end of treatment, including expected prednisone in the tapering regimen per protocol, add-on prednisone, CS used in rescue therapy and the use of commercial prednisone.
^{e}p-value from the Wilcoxon rank-sum test.
^{f}Disease remission is defined as the resolution of signs and symptoms of PMR, and normalization of CRP (<10 mg/L).
^{g}Flare is defined as either 1) recurrence of signs and symptoms attributable to active PMR plus an increase in CS dose due to PMR, or 2) elevation of ESR attributable to active PMR plus an increase in CS dose due to PMR.
^{h}The status of normalization of CRP from Week 12 through Week 52 was determined based on the CRP values measured at Week 16, Week 20, Week 24, Week 32, Week 40 and Week 52. If there were 2 or more consecutive visits with CRP ≥10 mg/L, then it was categorized as no normalization of CRP.
ⁱSuccessful adherence to the prednisone taper from Week 12 through Week 52 is defined as participants who did not take rescue therapy from Week 12 through Week 52 and might include the use of any excess prednisone (beyond the per protocol CS tapering regimen) with a cumulative dose of ≤100 mg (or corticosteroid dose equivalents), such as those employed to manage an AE not related to PMR. The cumulative dose of excess prednisone use was counted from baseline to Week 52.
^{j}Time (days) is calculated from randomization to first PMR flare after clinical remission up to Week 52.
^{k}Patients who never achieved remission were censored at the randomization day, and those who achieved clinical remission and never flared were censored at the end of treatment assessment date up to Week 52. The two-sides 95% CI was computed by the Brookmeyer and Crowley method (log transformation).
^{l}From the Cox proportional hazards model.
^{m}The analysis of covariance (ANCOVA) model includes the treatment groups and baseline glucocorticoid toxicity score as fixed effects.
ⁿType III sum of squares MMRM with PROC MIXED assuming an unstructured covariance structure: model = baseline, treatment, visit, and treatment-by-visit interaction, and baseline-by-visit interaction. Patients who had a baseline and at least 1 post-baseline value at Week 12, Week 24, or Week 52 were included in the MMRM analysis.
Note:
smaller cumulative worsening score implies less glucocorticoid toxicity. Smaller aggregate improvement score implies less glucocorticoid toxicity.

### Primary Efficacy Endpoint

The proportion of participants achieving sustained remission at Week 52 was higher in the sarilumab 200 mg q2w+14-week taper group compared to the placebo+52-week taper group. There were 17 (28.3%) participants in the sarilumab 200 mg q2w+14-week taper group and 6 (10.3%) participants in the placebo+52-week taper group who achieved sustained remission at Week 52 with a proportion difference of 18.0 (95% CI: 4.15, 31.82). The difference was statistically significant with a p-value of 0.0193, as shown in Table 9.

TABLE 9
Number (%) of patients achieving sustained
remission at Week 52 - ITT population

| | Placebo + | Sarilumab 200 mg | |
|---|---|---|---|
| | 52-week taper | q2w + 14-week taper | |
| | (n = 58) | (n = 60) | |
| Number | 58 | 60 | |
| Yes | 6 (10.3) | 17 | (28.3) |
| Proportion difference (95% CI) | | 18.0 | (4.15, 31.82) |

| | |
|---|---|
| for sarilumab vs placebo | |
| p-value^{a} | 0.0193 |

CI: confidence interval
^{a}p-value from Fisher's exact
Note:
Patients who did not achieve remission, received rescue treatment with open label prednisone (or equivalent), withdrew from the study before week 52, or had missing data that prevented assessment of the primary endpoint were considered as non-responders.

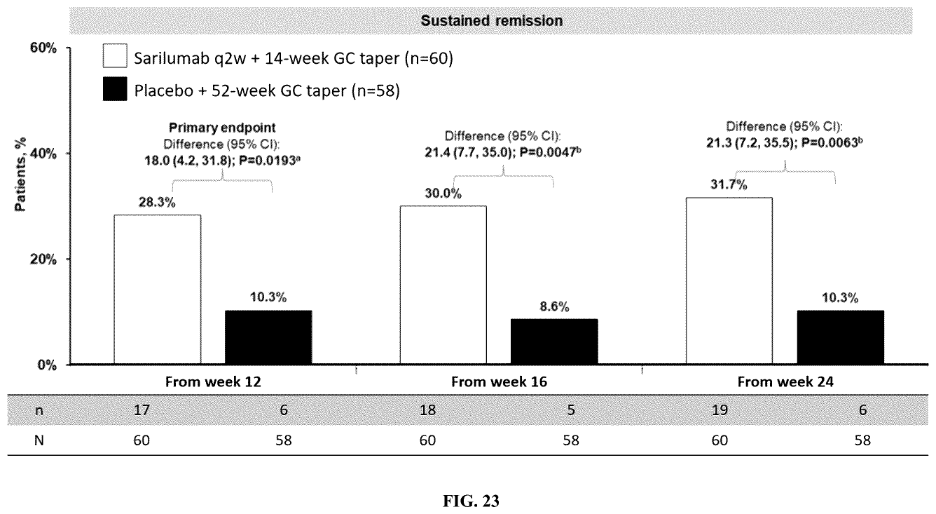

FIG. 23 also shows a higher proportion of patients in the sarilumab arm, versus the comparator, achieved sustained remission from weeks 16 to 52 (30.0% vs 8.6%; difference [95% CI]: 21.4 [7.7, 35.0]; P=0.0047) and from weeks 24 to 52 (31.7% vs 10.3%; difference [95% CI]: 21.3 [7.2, 35.5]; P=0.0063). This improvement in sustained remission rate in the sarilumab arm was due to additional responses seen between weeks 12 to 24.

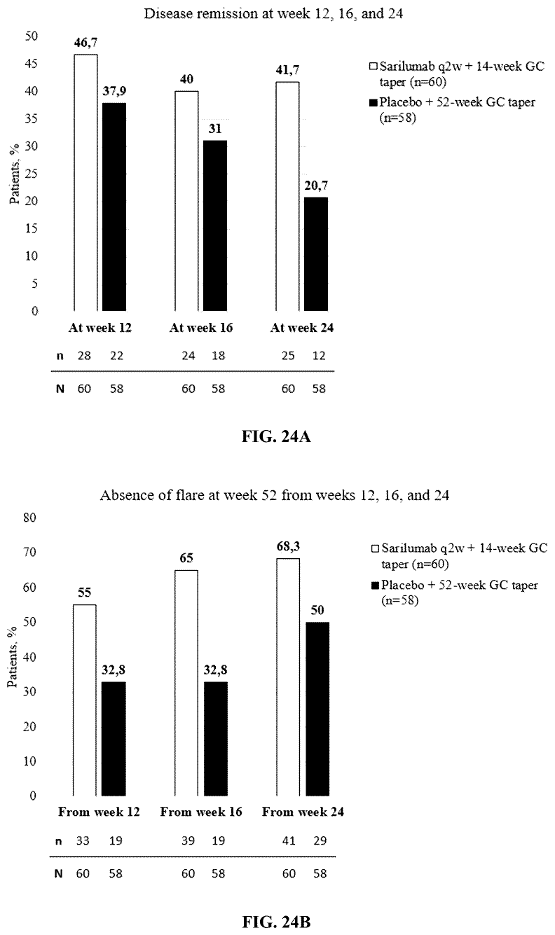

Similarly, all the independent components of the sustained remission were achieved by a higher proportion of patients in the sarilumab arm, versus the comparator arm, during each assessment period. As seen in FIG. 24, in both the sarilumab arm and the comparator arm, the disease remission rates decreased slightly from weeks 12 to 52 assessment (46.7% vs 37.9%) to weeks 16 to 52 (40.0% vs 31.0%) and weeks 24 to 52 (41.7% vs 20.7%). While disease remission declined, likely due to missing or abnormal CRP, the proportion of patients with no PMR signs and symptoms increased over time.

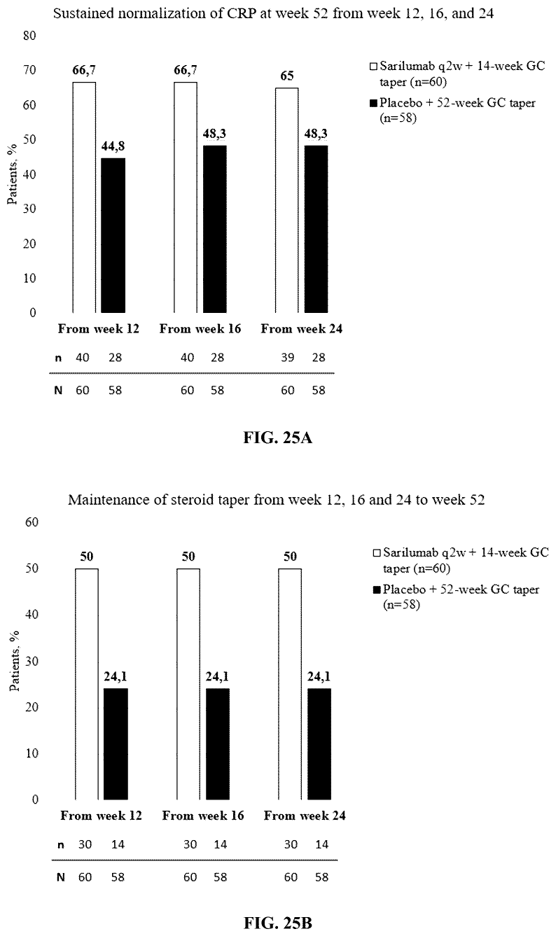

In the sarilumab arm, the proportion of patients with no disease flare increased from weeks 12 to 52 assessment to weeks 16 to 52 and weeks 24 to 52 assessments, whereas the proportion of patients with sustained CRP normalization or those who adhered to protocol-defined GC taper remained the same during the three assessment periods as seen in FIG. 25. Sensitivity analyses were performed for the primary endpoint. These analyses included removing the acute phase reactant CRP from the definition of sustained remission and also an additional analysis removing participants who discontinued study treatment due to an incorrect diagnosis of PMR.

As shown in Table 10, the proportion of participants achieving sustained remission at Week 52 excluding the acute phase reactant CRP continued to be greater in the sarilumab 200 mg q2w+14-week taper group compared to the placebo+52-week taper group (19 [31.7%] participants and 8 [13.8%] participants, respectively) with a proportion difference of 17.9 (95% CI: 3.13, 32.61). Consistent with the primary analysis, a statistically significant difference was observed in favor of the sarilumab 200 mg q2w+14-week taper group compared to the placebo+52-week taper group with a p-value of 0.0280.

TABLE 10
Sensitivity Analysis: Number (%) of patients
achieving sustained remission at Week 52 excluding
acute phase reactants - ITT population

| | Placebo + | Sarilumab 200 mg | |
|---|---|---|---|
| | 52-week taper | q2w + 14-week taper | |
| | (n = 58) | (n = 60) | |
| Number | 58 | 60 | |
| Yes | 8 (13.8) | 19 | (31.7) |
| Proportion difference (95% CI) | | 17.9 | (3.13, 32.61) |

| | |
|---|---|
| for sarilumab vs placebo | |
| p-value^{a} | 0.0280 |

CI: confidence interval
^{a}p-value from Fisher's exact
Note:
Patients who did not achieve remission, received rescue treatment with open label prednisone (or equivalent), withdrew from the study before week 52, or had missing data that prevented assessment of the primary endpoint were considered as non-responders.

Four participants were incorrectly diagnosed with PMR (2 participants in the sarilumab 200 mg q2w+14-week taper group and 2 participant in the placebo+52-week taper group). The proportion of participants achieving sustained remission at week 52 excluding those with an incorrect PMR diagnosis continued to be greater in the sarilumab 200 mg q2w+14-week taper group compared to the placebo+52-week taper group (17 [29.3%] participants and 6 [10.9%] participants, respectively) with a proportion difference of 18.4 (95% CI: 4.08, 32.72). As shown in Table 11, consistent with the primary analysis, a statistically significant difference was observed in favor of the sarilumab 200 mg q2w+14-week taper group compared to the placebo+52-week taper group with a p-value of 0.0193.

TABLE 11
Sensitivity Analysis: Number (%) of patients achieving
sustained remission at Week 52 excluding those
with incorrect PMR diagnosis - ITT population

| | Placebo + | Sarilumab 200 mg | |
|---|---|---|---|
| | 52-week taper | q2w + 14-week taper | |
| | (n = 58) | (n = 60) | |
| Number | 55 | 58 | |
| Yes | 6 (10.9) | 17 | (29.3) |
| Proportion difference (95% CI) | | 18.4 | (4.08, 32.72) |

| | |
|---|---|
| for sarilumab vs placebo | |
| p-value^{a} | 0.0193 |

CI: confidence interval
^{a}p-value from Fisher's exact
Note:
Patients who did not achieve remission, received rescue treatment with open label prednisone (or equivalent), withdrew from the study before week 52, or had missing data that prevented assessment of the primary endpoint were considered as non-responders.

Tipping point analysis was also conducted to assess the robustness of the conclusion of the primary endpoint analysis. Participants who did not achieve remission or who received rescue treatment were considered as non-responders. Participants who withdrew from the study before week 52 and did not experience a disease flare prior to withdrawal were considered as “missing” and were sequentially imputed for the tipping point analysis. As shown in Table 12, there were 13 participants from the sarilumab 200 mg q2w+14-week taper group and 9 participants from the placebo+52-week taper group considered as missing.

TABLE 12
Tipping point analysis: Patient accountability
at Week 52 - ITT population

| | Placebo + | Sarilumab 200 mg |
|---|---|---|
| Sustained remission at | 52-week taper | q2w + 14-week taper |
| week 52 (primary endpoint) | (n = 58) | (n = 60) |
| Completed treatment at | 36 (62.1) | 42 (70.0) |
| week 52 visit | | |
| Responders | 6 | 17 |
| Non-responders^{a} | 30 | 25 |
| Did not complete | 22 (37.9) | 18 (30.0) |
| treatment at week 52 visit | | |
| Non-responders^{b} | 13 | 5 |
| Missing^{c} | 9 | 13 |

^{a}Patients who did not achieve remission or receive rescue treatment with open label prednisone (or equivalent) are considered as non-responders.
^{b}Patients who received rescue treatment.
^{c}Patients who withdrew from the study before week 52 and did not experience a disease flare prior to withdrawal are considered as missing and be sequentially imputed for the tipping point analysis.

The results of the tipping point analysis showed in Table 13.

TABLE 13
Tipping point analysis: p-values for sustained remission at Week 52 - ITT population

| Number of | | | | | | | | | | |
|---|---|---|---|---|---|---|---|---|---|---|
| responders in | | | | | | | | | | |
| sarilumab | | | | | | | | | | |
| 200 mg + | | | | | | | | | | |
| 14-week taper | | | | | | | | | | |
| patients with | Number of responders in Placebo + 52-week taper patients with missing | | | | | | | | | |
| missing primary | primary endpoint | | | | | | | | | |
| endpoint | 0 | 1 | 2 | 3 | 4 | 5 | 6 | 7 | 8 | 9 |
| 0 | 0.0193 | 0.0389 | 0.0714 | 0.1207 | 0.1902 | 0.2817 | 0.3953 | 0.5287 | 0.6779 | 0.8372 |
| 1 | 0.0112 | 0.0236 | 0.0453 | 0.0799 | 0.1310 | 0.2015 | 0.2932 | 0.4059 | 0.5374 | 0.6840 |
| 2 | 0.0063 | 0.0140 | 0.0280 | 0.0515 | 0.0879 | 0.1404 | 0.2118 | 0.3035 | 0.4153 | 0.5451 |
| 3 | 0.0035 | 0.0081 | 0.0169 | 0.0324 | 0.0574 | 0.0953 | 0.1491 | 0.2212 | 0.3128 | 0.4237 |
| 4 | 0.0019 | 0.0046 | 0.0100 | 0.0198 | 0.0366 | 0.0631 | 0.1023 | 0.1571 | 0.2296 | 0.3211 |
| 5 | 0.0010 | 0.0025 | 0.0057 | 0.0119 | 0.0227 | 0.0407 | 0.0683 | 0.1087 | 0.1643 | 0.2372 |
| 6 | 0.0005 | 0.0014 | 0.0032 | 0.0069 | 0.0138 | 0.0256 | 0.0445 | 0.0733 | 0.1146 | 0.1708 |
| 7 | 0.0003 | 0.0007 | 0.0018 | 0.0039 | 0.0081 | 0.0157 | 0.0283 | 0.0482 | 0.0778 | 0.1199 |
| 8 | 0.0001 | 0.0004 | 0.0009 | 0.0022 | 0.0047 | 0.0094 | 0.0175 | 0.0308 | 0.0515 | 0.0820 |
| 9 | <.0001 | 0.0002 | 0.0005 | 0.0012 | 0.0026 | 0.0054 | 0.0105 | 0.0192 | 0.0333 | 0.0547 |
| 10 | <.0001 | <.0001 | 0.0002 | 0.0006 | 0.0014 | 0.0031 | 0.0062 | 0.0117 | 0.0209 | 0.0355 |
| 11 | <.0001 | <.0001 | 0.0001 | 0.0003 | 0.0008 | 0.0017 | 0.0035 | 0.0069 | 0.0128 | 0.0224 |
| 12 | <.0001 | <.0001 | <.0001 | 0.0002 | 0.0004 | 0.0009 | 0.0020 | 0.0040 | 0.0076 | 0.0138 |
| 13 | <.0001 | <.0001 | <.0001 | <.0001 | 0.0002 | 0.0005 | 0.0011 | 0.0022 | 0.0044 | 0.0083 |

As shown in Table 14, a post-hoc analysis was performed to assess the impact of methotrexate on the primary efficacy endpoint. There was no trend to suggest that methotrexate use impacted the primary endpoint.

TABLE 14
Post-hoc analysis: Summary of sustained remission at Week
52 for patients with concomitant use of methotrexate
during the 52-week treatment period - ITT population

| | Placebo + | | Sarilumab 200 mg | |
|---|---|---|---|---|
| | 52-week taper | | q2w + 14-week taper | |
| | (n = 58) | | (n = 60) | |
| Patients with concomitant use of | 17 | (29.3%) | 12 | (20.0%) |
| methotrexate during the treatment period | | | | |
| Sustained remission at week 52 | | | | |
| Responders | 6 | | 17 | |
| Patients with concomitant use of | 2/6 | (33.3%) | 3/17 | (17.6%) |
| methotrexate during the treatment | | | | |
| period | | | | |
| Non-responders | 52 | | 43 | |
| Patients with concomitant use of | | | | |
| methotrexate during the treatment | 15/52 | (28.8%) | 9/43 | (20.9%) |

period

Note:
Patients who did not achieve remission, received rescue treatment with open label prednisone (or equivalent), withdrew from the study before week 52, or had missing data that prevented assessment of the primary endpoint were considered as non-responders.

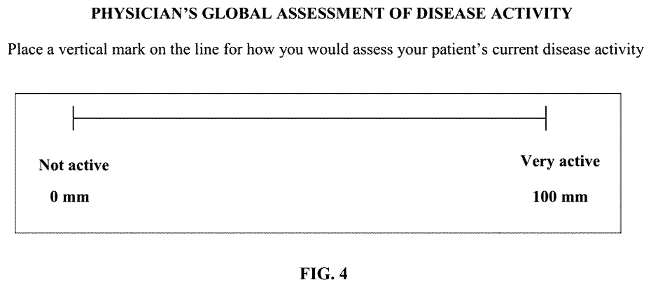

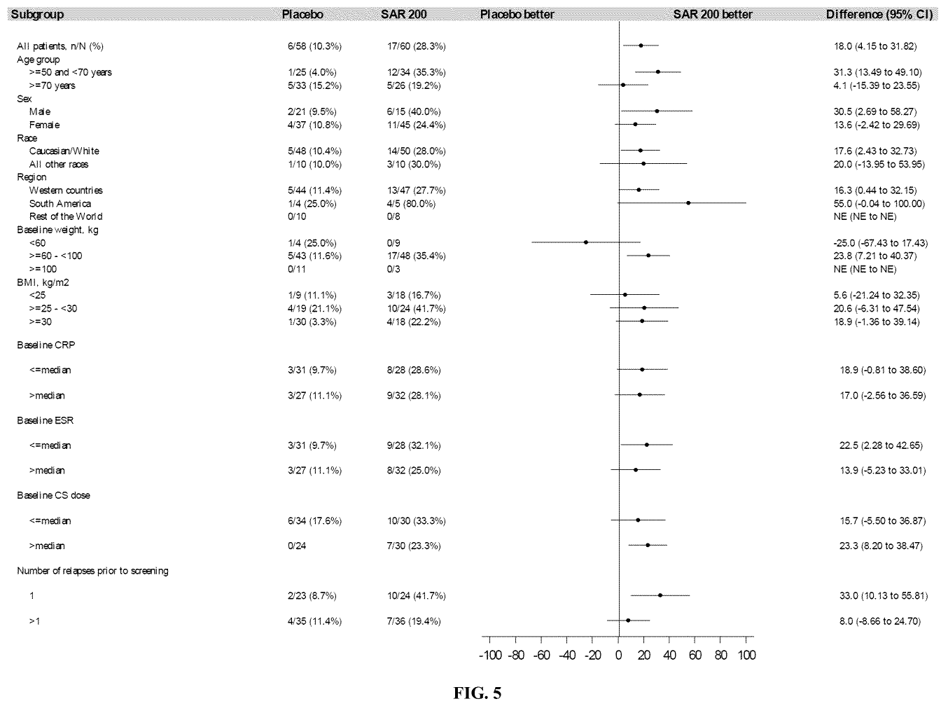

As shown in FIG. 5, subgroup analyses were performed for the primary endpoint to assess the consistency in treatment effects. Subgroup analysis of the primary endpoint results demonstrated a numerical trend in favor of the sarilumab 200 mg q2w+14-week taper group compared to the placebo+52-week taper group except in participants with a baseline weight <60 kg; however, the sample size was very small in this subgroup. There was no statistically significant interaction effect between each subgroup and treatment group. The sample size for some of the subgroups was small and the event counts were low.

### Secondary Efficacy Endpoints

### Components of Sustained Remission

As shown in Table 15, overall, there was a higher proportion of participants achieving each component of the sustained remission in the sarilumab 200 mg q2w+14-week taper group than in the placebo+52-week taper group. The proportion of participants who achieved disease remission within 12 weeks was higher in the sarilumab 200 mg q2w+14-week taper group compared to the placebo+52-week taper group (28 [46.7%] participants and 22 [37.9%] participants, respectively). The proportion of participants with absence of disease flare from week 12 through week 52 was higher in the sarilumab 200 mg q2w+14-week taper group compared to the placebo+52-week taper group (33 [55.0%] participants and 19 [32.8%] participants, respectively). The proportion of participants who achieved sustained reduction of CRP from week 12 through week 52 was higher in the sarilumab 200 mg q2w+14-week taper group compared to the placebo+52-week taper group (40 [66.7%] participants and 26 [44.8%] participants, respectively). The proportion of participants who achieved successful adherence to the prednisone taper from week 12 through week 52 was higher in the sarilumab 200 mg q2w+14-week taper group compared to the placebo+52-week taper group (30 [50.0%] participants and 14 [24.1%] participants, respectively).

TABLE 15
Components of sustained remission composite
measure at Week 52 - ITT population

| | | Sarilumab 200 mg |
|---|---|---|
| | Placebo + | q2w + 14-week |
| | 52-week taper | taper |
| | (n = 58) | (n = 60) |
| Achievement of disease remission | | |
| no later than week 12^{a} | | |
| Number | 58 | 60 |
| Yes | 22 (37.9) | 28 (46.7) |
| No | 36 (62.1) | 32 (53.3) |
| Absence of disease flare from | | |
| week 12 through week 52^{b} | | |
| Number | 58 | 60 |
| Yes | 19 (32.8) | 33 (55.0) |
| No | 39 (67.2) | 27 (45.0) |
| Sustained reduction of CRP from | 58 | 60 |
| week 12 through week 52^{c} | | |
| Number | 26 (44.8) | 40 (66.7) |
| Yes | 32 (55.2) | 20 (33.3) |
| No | | |
| Successful adherence to the | | |
| prednisone taper from week 12 | | |
| through week 52^{d} | | |
| Number | 58 | 60 |
| Yes | 14 (24.1) | 30 (50.0) |
| No | 44 (75.9) | 30 (50.0) |

^{a}Disease remission is defined as resolution of signs and symptoms of PMR. and normalization of CRP (<10 mg/L).
^{b}Flare is defined as either 1) recurrence of signs and symptoms attributable to active PMR plus an increase in CS dose due to PMR, or 2) elevation of ESR attributable to active PMR plus an increase in CS dose due to PMR.
^{c}The status of normalization of CRP from week 12 through week 52 was determined based on the CRP values measured at week 16, week 20, week 24, week 32, week 40 and week 52. If there were two or more consecutive visits with CRP >10 mg/L, then it was categorized as no normalization of CRP.
^{d}Successful adherence to the prednisone taper from week 12 through week 52 is defined as patients who did not take rescue therapy from week 12 through week 52 and might include the use of any excess prednisone (beyond the per protocol CS tapering regimen) with a cumulative dose of ≤100 mg (or equivalent), such as those employed to manage AE not related to PMR. The cumulative dose of excess prednisone use was counted from baseline to week 52.

As shown in Table 16, after the baseline visit, the number (%) of participants without any PMR signs and symptoms (excluding participants who had received rescue therapy) was higher at each visit in the sarilumab 200 mg q2w+14-week taper group compared to the placebo+52-week taper group. At the end of treatment (week 52), 27 (84.4%) participants in the sarilumab 200 mg q2w+14-week taper group and 8 (47.1%) participants in the placebo+52-week taper group did not have any PMR signs and symptoms (excluding participants who had received rescue therapy).

TABLE 16
Number (%) of patients without any PMR signs and symptoms by visit
excluding patients who had rescue therapy - ITT population

| | Placebo + | Sarilumab 200 mg |
|---|---|---|
| | 52-week taper | q2w + 14-week taper |
| | (n = 24) | (n = 41) |
| Baseline | | |
| Number | 24 | 41 |
| n (%) | 3 (12.5) | 5 (12.2) |
| V3/Week 2 | | |
| Number | 24 | 40 |
| n (%) | 7 (29.2 | 14 (35.0) |
| V4/Week 4 | | |
| Number | 24 | 39 |
| n (%) | 10 (41.7) | 18 (46.2) |
| V5/Week 8 | | |
| Number | 23 | 39 |
| n (%) | 8 (34.8) | 21 (53.8) |
| V6/Week 12 | | |
| Number | 20 | 36 |
| n (%) | 8 (40.0) | 24 (66.7) |
| V7/Week 16 | | |
| Number | 21 | 36 |
| n (%) | 9 (42.9) | 24 (66.7) |
| V8/Week 20 | | |
| Number | 20 | 34 |
| n (%) | 9 (45.0) | 20 (58.8) |
| V9/Week 24 | | |
| Number | 20 | 34 |
| n (%) | 8 (40.0) | 25 (73.5) |
| V10/Week 32 | | |
| Number | 18 | 32 |
| n (%) | 8 (44.4) | 25 (78.1) |
| V11/Week 40 | | |
| Number | 17 | 32 |
| n (%) | 9 (52.9) | 24 (75.0) |
| V12/Week 52 | | |
| Number | 17 | 32 |
| n (%) | 8 (47.1) | 27 (84.4) |

### Total Cumulative Corticosteroid (Including Prednisone) Dose

Table 17 presents the cumulative CS dose during the treatment period. The placebo+52-week taper group had a larger expected cumulative CS dose compared to the sarilumab 200 mg q2w+14-week taper group (52-week and 14-week CS taper, respectively). The median expected cumulative CS dose (prednisone or corticosteroid dose equivalents) over the 52-week treatment period was 777 mg in the sarilumab 200 mg q2w+14 weeks taper group, as compared to 2044 mg in the placebo+52-week taper group.

The placebo+52-week taper group had a larger actual cumulative CS dose due to the differences in the duration of the CS taper between the placebo+52-week taper group and the sarilumab 200 mg q2w+14-week taper group. The total median cumulative prednisone dose over the 52-week treatment period (i.e., actually taken by participants) was 777 mg in the sarilumab 200 mg q2w+14-week taper group, as compared to 2044 mg in the placebo+52-week taper group.

The median of the difference between the actual and expected cumulative CS dose was significantly lower in the sarilumab 200 mg q2w+14-week taper group compared to the placebo+52-week taper group (0.0 mg versus 199.5 mg; p=0.0189).

TABLE 17
Cumulative corticosteroid dose during
treatment period - ITT population

| | Placebo + | Sarilumab 200 mg |
|---|---|---|
| | 52-week taper | q2w + 14-week taper |
| | (n = 58) | (n = 60) |
| Expected cumulative | | |
| CS dose (mg)^{a} | | |
| Number | 58 | 59 |
| Mean (SD) | 1780.6 (478.3) | 741.9 (99.9) |
| SE | 62.81 | 13.00 |
| Median (95% CI) | 2044.0 | 777.0 |
| | (2031.0, 2044.0) | (777.0. 777.0) |
| Q1; Q3 | 1780.0: 2044.0 | 777.0; 777.0 |
| Min; Max | 404; 2044 | 224; 777 |
| Actual cumulative | | |
| CS dose (mg)^{b} | | |
| Number | 58 | 59 |
| Mean (SD) | 2235.8 (839.4) | 1039.5 (612.2) |
| SE | 110.21 | 79.70 |
| Median (95% CI) | 2044.0 | 777.0 |
| | (2044.0, 2455.0) | (777.0, 777.0) |
| Q1; Q3 | 1950.0; 2840.0 | 777.0; 1018.5 |
| Min; Max | 404; 3676 | 224; 2885 |
| p-value vs placebo^{c} | | <.0001 |
| Difference between actual | | |
| and expected cumulative | | |
| CS dose (mg) | | |
| Number | 58 | 59 |
| Mean (SD) | 455.1 (548.1) | 297.6 (588.7) |
| SE | 71.97 | 76.64 |
| Median (95% CI) | 199.5 | 0.0 |
| | (0.0, 496.0) | (0.0, 0.0) |
| Q1; Q3 | 0.0; 846.0 | 0.0; 245.0 |
| Min; Max | −57; 1632 | −13; 2108 |
| p-value vs placebo^{c} | | 0.0189 |

CS: corticosteroid; CI: confidence interval; SE: standard error
^{a}Expected cumulative dose based on CS tapering regimen up to the end of treatment, assuming that the taper was continued without error.
^{b}Cumulative dose of CS up to the end of treatment, including expected prednisone in tapering regimen add-on prednisone, CS used in rescue therapy and the use of commercial prednisone. per protocol, add-on prednisone, CS used in rescue therapy and the use of commercial prednisone.
^{c}p-value from Wilcoxon rank-sum test.

The majority of the difference between the actual and expected cumulative CS dose was due to the rescue therapy received by participants for PMR. As shown in Table 18, the cumulative number (%) of participants who received rescue therapy due to PMR was lower in the sarilumab 200 mg q2w+14-week taper group compared to the placebo+52-week taper group. During the 52-week treatment period, there were 34 (58.6%) participants in the placebo+52-week taper group and 19 (32.2%) participants in the sarilumab 200 mg q2w+14-week taper group who received rescue therapy due to PMR.

TABLE 18
Cumulative number (%) of patients who had
rescue therapy due to PMR - ITT population

| | Placebo + | Sarilumab 200 mg |
|---|---|---|
| During 52-week | 52-week taper | q2w + 14-week taper |
| treatment period | (n = 58) | (n = 60) |
| Number | 58 | 59^{a} |
| Had rescue therapy | 34 (58.6) | 19 (32.2) |

^{a}One patient who was randomized but not treated was excluded.

### Time to First PMR Flare

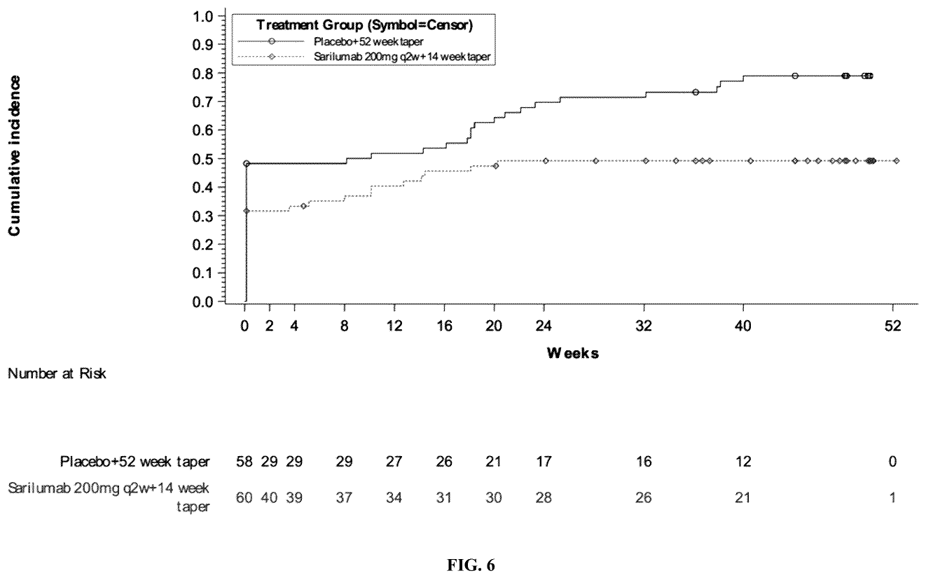

FIG. 6 provides Kaplan-Meier estimates for time to first PMR flare after clinical remission up to 52 weeks. The time to first PMR flare was statistically significantly longer in sarilumab treated participants than in placebo-treated participants, and sarilumab treated participants never reached the median. There were 41 (68.3%) participants in the sarilumab 200 mg q2w+14-week taper group and 30 (51.7%) participants in the placebo+52-week taper group who achieved clinical remission during the 52-week treatment period. Participants in the sarilumab 200 mg q2w+14-week taper group were less likely to have a PMR flare after achieving clinical remission compared to participants in the placebo+52-week taper group (16.7% versus 29.3%) with a hazard ratio of 0.56 (95% CI: 0.35 to 0.90; p=0.0153). The median value was not reached in the sarilumab 200 mg q2w+14-week taper group. A post-hoc analysis was conducted for time to first PMR flare after the clinical remission endpoint to analyze the total time to first PMR flare calculated since first clinical remission. The post-hoc analysis results are consistent with the analyses for total time to first PMR flare calculated from the randomization day.

### Glucocorticoids Toxicity Index-Cumulative Worsening Score (CWS) and Aggregate Improvement Score (AIS)

As shown in Table 19, there was a numerical trend towards sarilumab 200 mg q2w+14-week taper group to have lesser cumulative worsening and greater aggregate improvement compared to the placebo+52-week taper group. However, there was no significant difference in CWS between the sarilumab 200 mg q2w+14-week taper group and the placebo+52-week taper group with LS mean difference (SE) versus placebo was −4.90 (9.375) (95% CI: −23.477, 13.669; p=0.6020).

TABLE 19
Summary of cumulative worsening score during
52-week treatment period - ITT population

| | Placebo + | | Sarilumab 200 mg | |
|---|---|---|---|---|
| | 52-week taper | | q2w + 14-week taper | |
| | (n = 58) | | (n = 60) | |
| Number | 57 | | 60 | |
| Mean (SD) | 57.9 | (51.9) | 51.7 | (48.4) |
| SE | 6.87 | | 6.25 | |
| Median | 55.0 | | 39.0 | |
| Min; Max | 0; 257 | | 0; 195 | |
| LS mean (SE)^{a} | 57.22 | (6.678) | 52.32 | (6.507) |
| LS mean difference (SE) vs | | | −4.90 | (9.375) |

| | |
|---|---|
| placebo^{a} | |
| 95% CI | −23.477, 13.669 |
| p-value | 0.6020 |

CI: confidence interval: LS: least square; SD: standard deviation; SE: standard error of the mean Smaller cumulative worsening score implies less glucocorticoid toxicity.
^{a}Analysis of covariance (ANOVA) model includes treatment groups and baseline glucocorticoid toxicity score as fixed effects.

Similarly, as shown in Table 20, there was no significant difference in AIS between the sarilumab 200 mg q2w+14-week taper group and the placebo+52-week taper group with LS mean deference (SE) versus placebo was −6.59 (8.811) (95% CI: −24.047,10.862; p=0.4559).

TABLE 20
Summary of aggregate improvement score during
52-week treatment period - ITT population

| | Placebo + | | Sarilumab 200 mg | |
|---|---|---|---|---|
| | 52-week taper | | q2w + 14-week taper | |
| | (n = 58) | | (n = 60) | |
| Number | 57 | | 60 | |
| Mean (SD) | −0.5 | (57.1) | −1.1 | (44.6) |
| SE | 7.56 | | 5.76 | |
| Median | 0.0 | | 0.5 | |
| Min; Max | −283; 122 | | −122; 86 | |
| LS mean (SE)^{a} | 2.57 | (6.275) | −4.02 | (6.115) |

| | | |
|---|---|---|
| LS mean difference (SE) vs | −6.59 | (8.811) |

| | |
|---|---|
| placebo^{a} | |
| 95% CI | −24.047, 10.862 |
| p-value | 0.4559 |

CI: confidence interval: LS: least square; SD: standard deviation; SE: standard error of the mean Smaller cumulative worsening score implies less glucocorticoid toxicity.
^{a}Analysis of covariance (ANOVA) model includes treatment groups and baseline glucocorticoid toxicity score as fixed effects.

### Outcomes and Other Endpoints and Assessments

### PMR Activity Score

As shown in Table 21, the sarilumab+14-week taper group had higher proportions of participants with a low disease activity level over time, as compared to the placebo+52-week taper group. There were no participants in the sarilumab+14-week taper group with high levels of PMR activity at week 24 and week 52, and a higher proportion of participants were in the low disease activity level in the sarilumab+14-week taper group compared to placebo+52-week taper group.

TABLE 21
Post-hoc analysis: Number (%) of patients with high, medium and low levels of
PMR activity over time - ITT population

| | | | | Sarilumab 200 mg q2w + 14-week taper | | |
|---|---|---|---|---|---|---|
| | Placebo + 52-week taper (n = 58) | | | (n = 60) | | |
| | Low | Medium | High | Low | Medium | High |
| Baseline | 7 (12.1) | 14 (24.1) | 31 (53.4) | 7 (11.7) | 13 (21.7) | 29 (48.3) |
| V6/Week 12 | 18 (31.0) | 9 (15.5) | 6 (10.3) | 21 (35.0) | 8 (13.3) | 5 (8.3) |
| V9/Week 24 | 20 (34.5) | 10 (17.2) | 9 (15.5) | 24 (40.0) | 8 (13.3) | 0 |
| V12/Week 52 | 13 (22.4) | 13 (22.4) | 3 (5.2) | 26 (43.3) | 9 (15.0) | 0 |

PMR activity level is defined as low for PMR activity score less than 7, medium for PMR activity score between 7 to 17, and high for PMR activity score greater than 17.

As shown in Table 22, participants in the sarilumab 200 mg q2w+14-week taper group had a greater change from baseline in PMR-AS at Week 52 compared to the placebo+52—week taper group (LS mean [SE] was −15.57 [0.94] and −10.27 [0.97], respectively). The LS mean difference of PMR-AS in the sarilumab 200 mg q2w+14-week taper group versus the placebo+52-week taper group was statistically significant (−5.302, 95% CI: −8.006 to −2.597; p=0.0002).

TABLE 22
Change from baseline in PMR activity
score at week 52 - ITT population

| | Placebo + | | Sarilumab 200 mg | |
|---|---|---|---|---|
| | 52-week taper | | q2w + 14-week taper | |
| PMR-AS | (n = 58) | | (n = 60) | |
| Number^{a} | 28 | | 31 | |
| Baseline mean (SD) | 20.82 | (28.46) | 17.55 | (8.65) |
| Week 52 mean (SD) | 9.11 | (6.88) | 4.03 | (3.29) |
| Number^{b} | 41 | | 29 | |
| Change mean (SD) | −11.71 | (29.97) | −13.52 | (8.68) |
| LS mean (SE) | −10.27 | (0.97) | −15.57 | (0.94) |

| LS mean difference | −5.302 |
|---|---|
| (95% CI) | (−8.006, −2.597) |
| p-value vs placebo^{c} | 0.0002 |

^{a}Number of patients with assessment at both baseline and week 52.
^{b}Number of patients included in the MMRM analysis, which includes patients who had baseline and at least one post-baseline value at week 12, week 24, or week 52.
^{c}Type III sum of squares MMRM with PROC MIXED assuming an unstructured covariance structure: model = baseline, treatment, visit, and treatment-by-visit interaction, and baseline-by-visit interaction. All assessments were set to missing from the time a patient discontinued study medication early. No imputation was used for missing values.

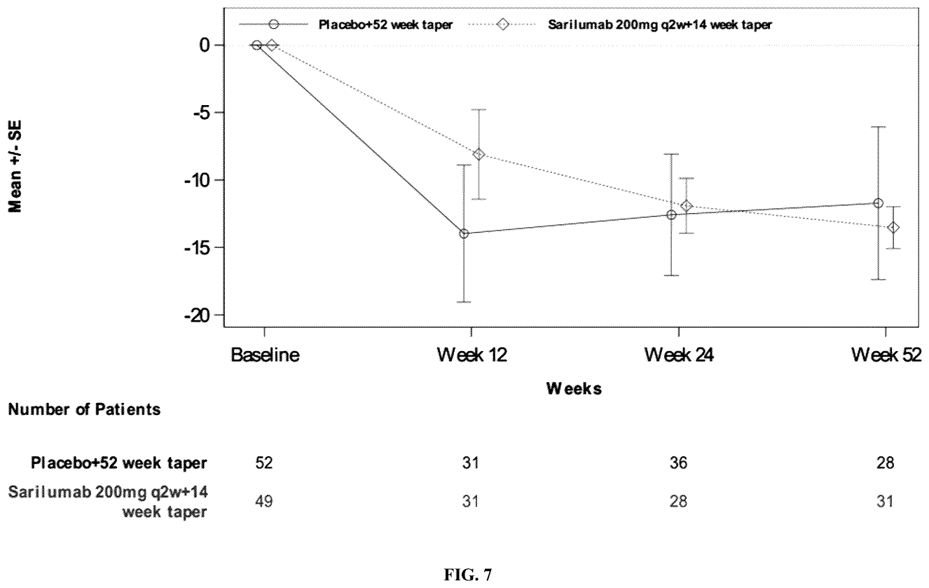

As shown in FIG. 7, the greatest reduction in PMR activity for both the sarilumab 200 mg q2w+14-week taper group and the placebo+52-week taper groups was seen from baseline to week 12 with reduction in the sarilumab 200 mg q2w+14-week taper group continuing until week 52.

### MD-VAS

As shown in Table 23, participants in the sarilumab 200 mg q2w+14-week taper group had a greater reduction from baseline in MD-VAS at week 52 compared to the placebo+52-week taper group (LS mean [SE] of −40.58 [3.40] and −30.49 [3.46], respectively). The LS mean difference of MD-VAS in the sarilumab 200 mg q2w+14-week taper group versus the placebo+52-week taper group was statistically significant (−10.097, 95% CI: −19.775, −0.419; p=0.0411).

TABLE 23
Change from baseline in MD-VAS at week 52 - ITT population

| | Placebo + | Sarilumab 200 mg |
|---|---|---|
| | 52-week taper | q2w + 14-week taper |
| MD-VAS | (n = 58) | (n = 60) |
| Number^{a} | 31 | 33 |

| | | | | |
|---|---|---|---|---|
| Baseline mean (SD) | 48.32 | (24.78) | 55.03 | (16.96) |
| Week 52 mean (SD) | 18.97 | (22.86) | 10.21 | (15.44) |

| | | |
|---|---|---|
| Number^{b} | 46 | 42 |

| | | | | |
|---|---|---|---|---|
| Change mean (SD) | −29.35 | (28.91) | −44.82 | (20.62) |
| LS mean (SE) | −30.49 | (3.46) | −40.58 | (3.40) |

| LS mean difference | −10.097 |
|---|---|
| (95% CI) | (−19.775, −0.419) |
| p-value vs placebo^{c} | 0.0411 |

^{a}Number of patients with assessment at both baseline and week 52.
^{b}Number of patients included in the MMRM analysis, which includes patients who had baseline and at least one post-baseline value at week 12, week 24, or week 52.
^{c}Type III sum of squares MMRM with PROC MIXED assuming an unstructured covariance structure: model = baseline, treatment, visit, and treatment-by-visit interaction, and baseline-by-visit interaction. All assessments were set to missing from the time a patient discontinued study medication early. No imputation was used for missing values.

### FACIT-Fatigue

As shown in Table 24, the change from baseline in the FACIT-Fatigue scale at week 52 was numerically higher in the sarilumab 200 mg q2w+14-week taper group compared to the placebo+52-week taper group (LS mean [SE] of 7.91 [1.33] and 4.17 [1.42], respectively). The LS mean difference of the FACIT-Fatigue scale in the sarilumab 200 mg q2w+14-week taper group versus the placebo+52-week taper group was 3.740 (95% CI: −0.108, 7.588; p=0.0567).

TABLE 24
Change from baseline in FACIT-Fatigue
at week 52 - ITT population

| | Placebo + | Sarilumab 200 mg |
|---|---|---|
| | 52-week taper | q2w + 14-week taper |
| FACIT-Fatigue (0-52) | (n = 58) | (n = 60) |
| Number^{a} | 36 | 42 |

| | | | | |
|---|---|---|---|---|
| Baseline mean (SD) | 31.97 | (11.92) | 32.81 | (10.66) |
| Week 52 mean (SD) | 36.78 | (10.87) | 40.74 | (9.29) |

| | | |
|---|---|---|
| Number^{b} | 54 | 52 |

| | | | | |
|---|---|---|---|---|
| Change mean (SD) | 4.81 | (10.95) | 7.93 | (9.55) |
| LS mean (SE) | 4.17 | (1.42) | 7.91 | (1.33) |

| LS mean difference | 3.740 |
|---|---|
| (95% CI) | (−0.108, 7.588) |
| p-value vs placebo^{c} | 0.0567 |

^{a}Number of patients with assessment at both baseline and week 52.
^{b}Number of patients included in the MMRM analysis, which includes patients who had baseline and at least one post-baseline value at week 12, week 24, or week 52.
^{c}Type III sum of squares MMRM with PROC MIXED assuming an unstructured covariance structure: model = baseline, treatment, visit, and treatment-by-visit interaction, and baseline-by-visit interaction. All assessments were set to missing from the time a patient discontinued study medication early. No imputation was used for missing FACIT-Fatigue scores.

The percent of patients treated with sarilumab and placebo that reported an improvement greater than or equal to MCID for FACIT-F score at week 52 is shown in FIG. 12. MCID for FACIT-F was an improvement greater than or equal to 4.0. These results show that sarilumab treatment led to numerically greater improvements in FACIT-F score than treatment with placebo (with a higher score representing better functioning or less fatigue).

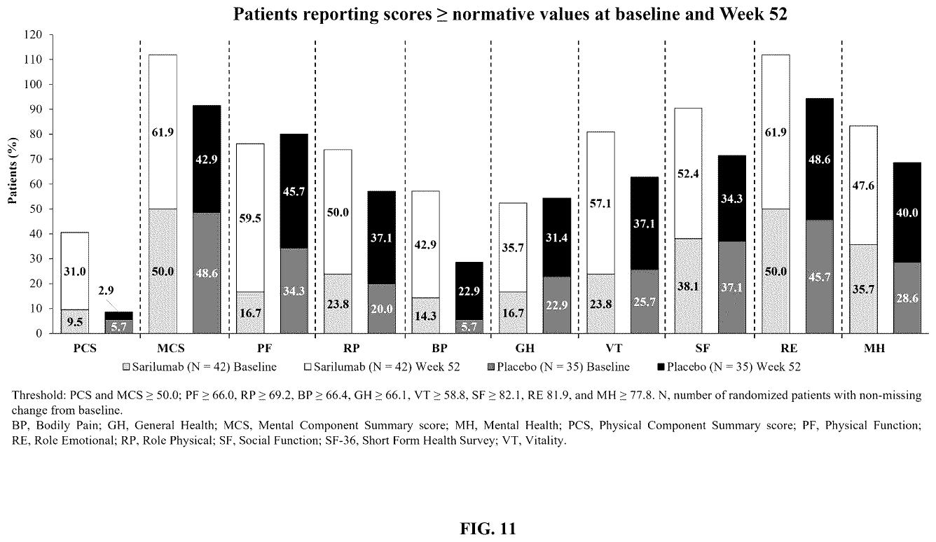

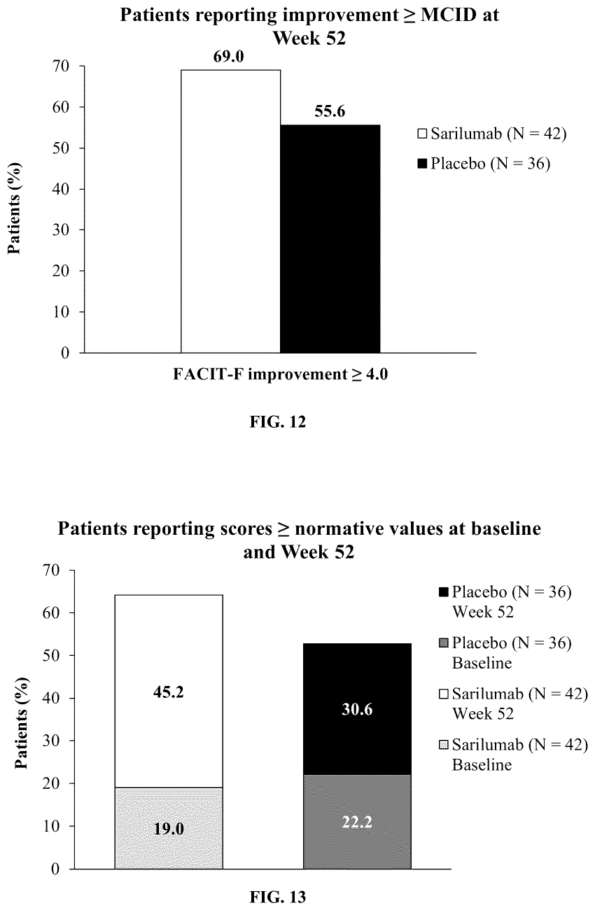

The percent of patients treated with sarilumab and placebo that reported FACIT-F scores greater than or equal to normative values at baseline and at week 52 is shown in FIG. 13. The threshold for normative values was greater than or equal to 43.5. These results show that sarilumab treatment led to numerically greater improvements in FACIT-F score than treatment with placebo.

### EQ-5D-3L

As shown in Table 25, participants in the sarilumab 200 mg q2w+14-week taper group had a greater increase from baseline in the EQ-5D-3L single index utility score at week 52 compared to placebo+52-week taper group (LS mean [SE] of 0.11 [0.04] and −0.02 [0.04], respectively). The LS mean difference of the EQ-5D-3L single index utility score in the sarilumab 200 mg q2w+14-week taper group versus the placebo+52-week taper group was statistically significant (0.130, 95% CI: 0.010, 0.250; p=0.0336).

TABLE 25
Change from baseline in EQ-5D single index
utility at week 52 - ITT population

| | Placebo + | | Sarilumab 200 mg | |
|---|---|---|---|---|
| | 52-week taper | | q2w + 14-week taper | |
| EQ-5D - single index utility | (n = 58) | | (n = 60) | |
| Number^{a} | 35 | | 42 | |
| Baseline mean (SD) | 0.62 | (0.29) | 0.59 | (0.27) |
| Week 52 mean (SD) | 0.63 | (0.30) | 0.72 | (0.23) |
| Number^{b} | 54 | | 51 | |
| Change mean (SD) | 0.00 | (0.38) | 0.14 | (0.31) |
| LS mean (SE) | −0.02 | (0.04) | 0.11 | (0.04) |

| LS mean difference | 0.130 |
|---|---|
| (95% CI) | (0.010, 0.250) |
| p-value vs placebo^{c} | 0.0336 |

^{a}Number of patients with assessment at both baseline and week 52.
^{b}Number of patients included in the MMRM analysis, which includes patients who had baseline and at least one post-baseline value at week 12, week 24, or week 52.
^{c}Type III sum of squares MMRM with PROC MIXED assuming an unstructured covariance structure: model = baseline, treatment, visit, and treatment-by-visit interaction, and baseline-by-visit interaction. All assessments were set to missing from the time a patient discontinued study medication early. No imputation was used for missing EQ-5D scores.

As shown in Table 26, participants in the sarilumab 200 mg q2w+14-week taper group had a greater increase from baseline in the EQ-5D-3L VAS score at week 52 compared to placebo+52-week taper group (LS mean [SE] of 8.37 [3.46] and −0.46 (3.68), respectively). The LS mean difference of the EQ-5D-3L VAS score in the sarilumab 200 mg q2w+14-week taper group versus the placebo+52-week taper group was 8.830 (95% CI: −1.191, 18.850; p=0.0835).

TABLE 26
Change from baseline in EQ-5D VAS at Week 52 - ITT population

| | | |
|---|---|---|
| | Placebo + | Sarilumab 200 mg |
| | 52-week taper | q2w + 14-week taper |
| EQ-5D - VAS | (n = 58) | (n = 60) |

| Number^{a} | 35 | | 42 | |
|---|---|---|---|---|
| Baseline mean (SD) | 58.60 | (19.83) | 61.19 | (20.43) |
| Week 52 mean (SD) | 61.94 | (22.13) | 68.95 | (23.19) |
| Number^{b} | 54 | | 51 | |
| Change mean (SD) | 3.34 | (26.85) | 7.76 | (29.37) |
| LS mean (SE) | −0.46 | (3.68) | 8.37 | (3.46) |

| LS mean difference | 8.830 |
|---|---|
| (95% CI) | (−1.191, 18.850) |
| p-value vs placebo^{c} | 0.0835 |

^{a}Number of patients with assessment at both baseline and week 52.
^{b}Number of patients included in the MMRM analysis, which includes patients who had baseline and at least one post-baseline value at week 12, week 24, or week 52.
^{c}Type III sum of squares MMRM with PROC MIXED assuming an unstructured covariance structure: model = baseline, treatment, visit, and treatment-by-visit interaction, and baseline-by-visit interaction. All assessments were set to missing from the time a patient discontinued study medication early. No imputation was used for missing EQ-5D scores.

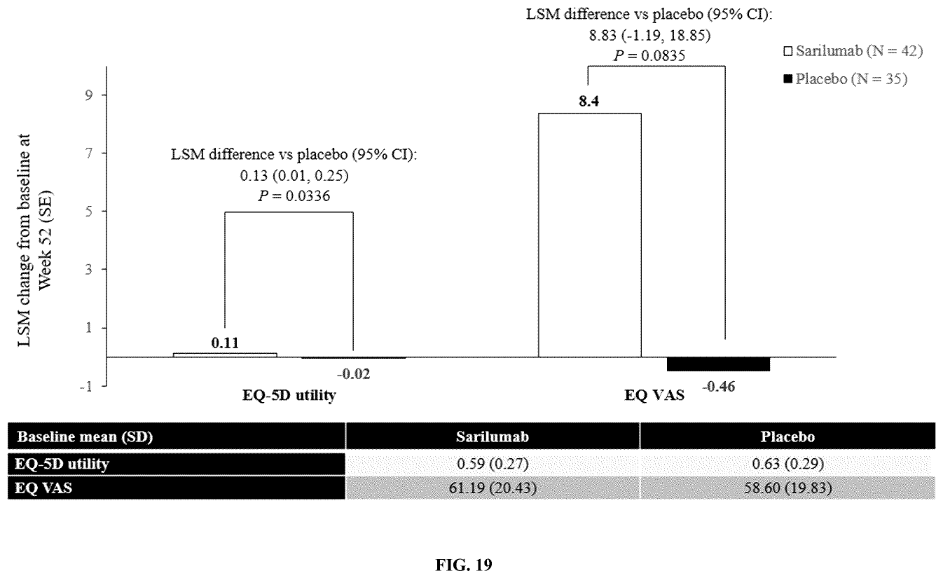

The LSM change from baseline at week 52 for EQ-5D index utility score and EQ VAS score for patients treated with sarilumab and placebo is shown in FIG. 19. These results show that sarilumab treatment led to a statistically greater improvement from baseline in EQ-5D index utility score with a LSM difference versus placebo of 0.13 (P=0.0336). These results also show that sarilumab treatment led to a numerically greater improvement from baseline in EQ VAS score (with a higher score indicating better health).

### SF-36

As shown in Table 27, participants in the sarilumab 200 mg q2w+14-week taper group had a greater increase from baseline in the PCS at week 52 compared to the placebo+52-week taper group (LS mean [SE] of 7.65 [1.34] and 2.87 [1.45], respectively). As shown in FIG. 8, the LS mean difference in PCS in the sarilumab 200 mg q2w+14-week taper group versus the placebo+52-week taper group was statistically significant (4.784, 95% CI: 0.865, 8.703; p=0.0172).

TABLE 27
Change from baseline in SF-36 physical component
summary score at week 52 - ITT population

| | Placebo + | | Sarilumab 200 mg | |
|---|---|---|---|---|
| SF-36 - physical component | 52-week taper | | q2w + 14-week taper | |
| summary score | (n = 58) | | (n = 60) | |
| Number^{a} | 35 | | 42 | |
| Baseline mean (SD) | 36.36 | (8.38) | 35.76 | (8.58) |
| Week 52 mean (SD) | 39.44 | (8.11) | 43.67 | (9.99) |
| Number^{b} | 54 | | 52 | |
| Change mean (SD) | 3.08 | (10.77) | 7.91 | (10.04) |
| LS mean (SE) | 2.87 | (1.45) | 7.65 | (1.34) |

| LS mean difference | 4.784 |
|---|---|
| (95% CI) | (0.865, 8.703) |
| p-value vs placebo^{c} | 0.0172 |

^{a}Number of patients with assessment at both baseline and week 52.
^{b}Number of patients included in the MMRM analysis, which includes patients who had baseline and at least one post-baseline value at week 12, week 24, or week 52.
^{c}Type III sum of squares MMRM with PROC MIXED assuming an unstructured covariance structure: model = baseline, treatment, visit, and treatment-by-visit interaction, and baseline-by-visit interaction. All assessments were set to missing from the time a patient discontinued study medication early. No imputation was used for missing SF-36 scores.

As shown in Table 28, Participants in the sarilumab 200 mg q2w+14-week taper group had a greater increase from baseline in the MCS at week 52 compared to the placebo+52-week taper group (LS mean [SE] of 3.04 [1.47] and −1.71 [1.57], respectively). As shown in FIG. 8, the LS mean difference of MCS score in the sarilumab 200 mg q2w+14-week taper group versus the placebo+52-week taper group was statistically significant (4.748, 95% CI: 0.484, 9.013; p=0.0295).

TABLE 28
Change from baseline in SF-36 mental component
summary score at week 52 - ITT population

| | Placebo + | | Sarilumab 200 mg | |
|---|---|---|---|---|
| SF-36 - mental component | 52-week taper | | q2w + 14-week taper | |
| summary score | (n = 58) | | (n = 60) | |
| Number^{a} | 35 | | 42 | |
| Baseline mean (SD) | 46.25 | (13.26) | 47.51 | (11.69) |
| Week 52 mean (SD) | 46.81 | (12.39) | 50.83 | (9.75) |
| Number^{b} | 54 | | 52 | |
| Change mean (SD) | 0.56 | (12.80) | 3.32 | (9.84) |
| LS mean (SE) | −1.71 | (1.57) | 3.04 | (1.47) |

| LS mean difference | 4.748 |
|---|---|
| (95% CI) | (0.484, 9.013) |
| p-value vs placebo^{c} | 0.0295 |

^{a}Number of patients with assessment at both baseline and week 52.
^{b}Number of patients included in the MMRM analysis, which includes patients who had baseline and at least one post-baseline value at week 12, week 24, or week 52.
^{c}Type III sum of squares MMRM with PROC MIXED assuming an unstructured covariance structure: model = baseline, treatment, visit, and treatment-by-visit interaction, and baseline-by-visit interaction. All assessments were set to missing from the time a patient discontinued study medication early. No imputation was used for missing SF-36 scores.

Table 29 provides the LSM change from baseline at week 52 for sarilumab versus placebo in each of the SF-36 domains including physical function (PF), role physical (RP), bodily pain (BP), general health (GH), vitality (VT), social function (SF), role emotional (RE), and mental health (MH). As illustrated in FIG. 9, 5 out of 8 domains of SF-36 showed statistically significantly greater improvement with sarilumab versus placebo (MH, RP, BP, SF, and VT).

TABLE 29
LSM for SF-36 Domains for Sarilumab and Placebo Treatment

| | LSM change from baseline at Week 52 (SE) | | | | LSM difference vs | |
|---|---|---|---|---|---|---|
| SF-36 domains | Sarilumab | | Placebo | | placebo (95% CI) | |
| PF | 13.68 | (3.59) | 6.16 | (3.84) | 7.52 | (−2.90, 17.95) |
| RP | 21.10 | (3.56) | 3.83 | (3.84) | 17.27 | (6.89, 27.64) |
| BP | 19.66 | (3.40) | 5.73 | (3.70) | 13.93 | (3.97, 23.90) |
| GH | 8.14 | (2.41) | 1.74 | (2.61) | 6.40 | (−0.65, 13.45) |
| VT | 17.04 | (3.02) | 2.49 | (3.28) | 14.55 | (5.69, 23.41) |
| SF | 8.62 | (4.02) | −4.29 | (4.35) | 12.91 | (1.16, 24.65) |
| RE | 6.26 | (3.56) | −1.56 | (3.76) | 7.8 | (−2.47, 18.12) |
| MH | 7.32 | (2.39) | 0.12 | (2.54) | 7.2 | (0.27, 14.13) |

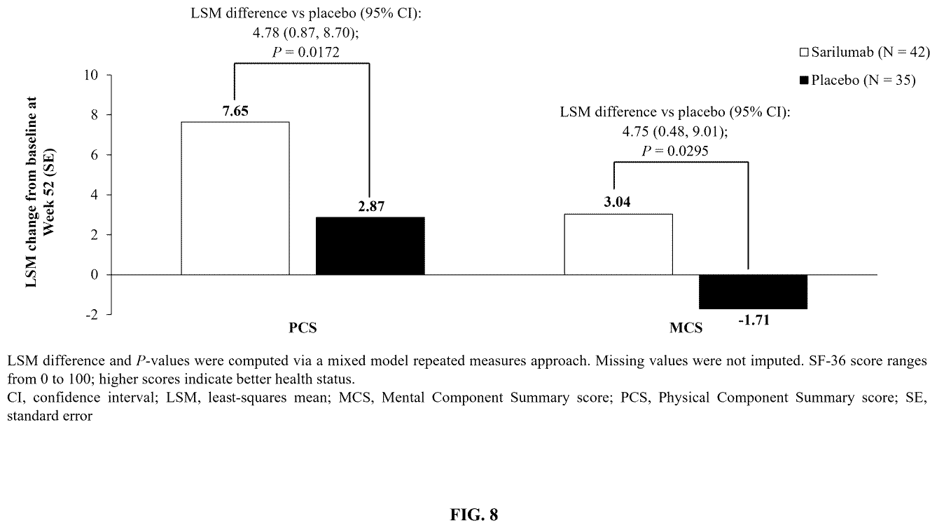

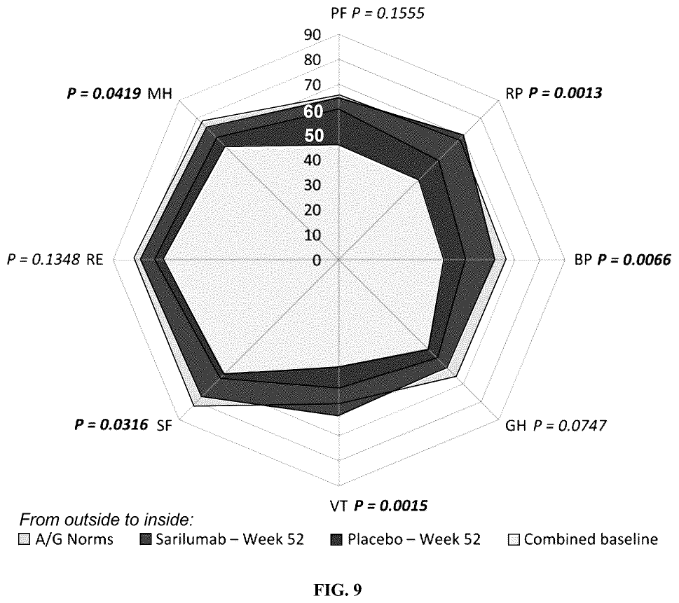

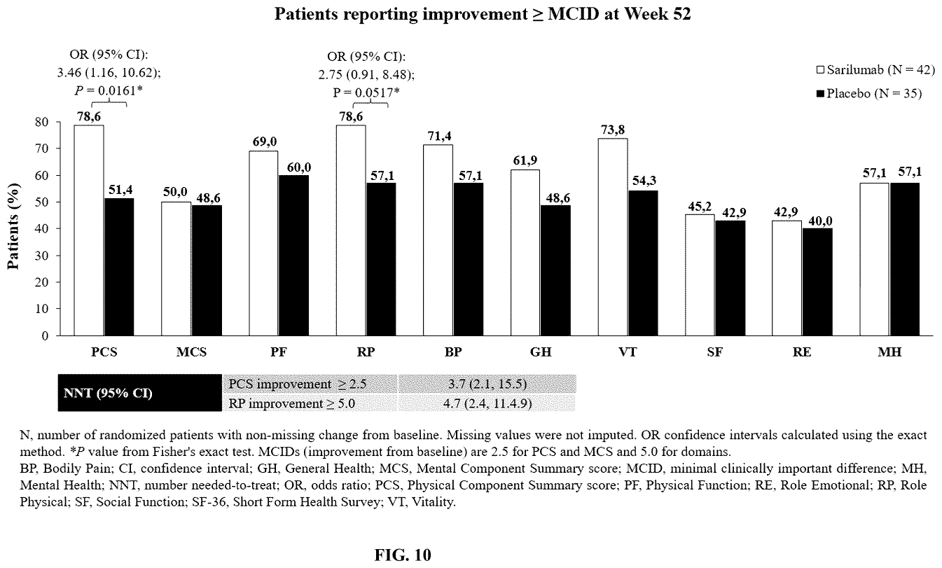

The percent of patients reporting an improvement greater than or equal to MCID at week 52 for patients receiving sarilumab and placebo for various SF-36 metrics including PCS, MCS, PF, RP, BP, GH, VT, SF, RE, and MH is presented in FIG. 10. A more patients receiving sarilumab reported improvements of greater than or equal to MCID at a statistically significant level for PCS (P=0.0161) and 5 of 8 SF-36 domain scores. MCID (improvement from baseline) was 2.5 for PCS and MCS. MCID was 5.0 for individual SF-36 domains.

The percent of patients treated with sarilumab and placebo that reported scores greater than or equal to normative values at baseline and at week 52 for various SF-36 metrics including PCS, MCS, PF, RP, BP, GH, VT, SF, RE, and MH is presented in FIG. 11. The threshold values are as follows: PCS and MCS≥50.0; PF≥66.0, RP≥69.2, BP≥66.4, GH≥66.1, VT≥58.8, SF≥82.1, RE 81.9, and MH≥77.8. These results show that numerically more patients receiving sarilumab reported scores greater than or equal to normative values in SF-36 MCS and in 4 SF-36 domain scores.

### HAQ-DI

As shown in Table 30, participants in the sarilumab 200 mg q2w+14-week taper group had a numerically greater reduction from baseline in the HAQ-DI standardized score at Week 52 compared to the placebo+52-week taper group (LS mean [SE] of −0.39 [0.09] and −0.15 [0.09], respectively). The LS mean difference in the HAQ-DI standardized score in the sarilumab 200 mg q2w+14-week taper group versus the placebo+52-week taper group was −0.246 (95% CI; −0.496, 0.005; p=0.0543).

TABLE 30
Change from baseline in HAQ-DI standardized
score at Week 52 - ITT population

| | Placebo + | | Sarilumab 200 mg | |
|---|---|---|---|---|
| | 52-week taper | | q2w + 14-week taper | |
| HAQ-DI standardized score | (n = 58) | | (n = 60) | |
| Number^{a} | 35 | | 42 | |
| Baseline mean (SD) | 0.95 | (0.77) | 1.14 | (0.69) |
| Week 52 mean (SD) | 0.76 | (0.65) | 0.69 | (0.64) |
| Number^{b} | 54 | | 51 | |
| Change mean (SD) | −0.20 | (0.65) | −0.45 | (0.68) |
| LS mean (SE) | −0.15 | (0.09) | −0.39 | (0.09) |

| LS mean difference | −0.246 |
|---|---|
| (95% CI) | (−0.496, 0.005) |
| p-value vs placebo^{c} | 0.0543 |

^{a}Number of patients with assessment at both baseline and week 52.
^{b}Number of patients included in the MMRM analysis, which includes patients who had baseline and at least one post-baseline value at week 12, week 24, or week 52.
^{c}Type III sum of squares MMRM with PROC MIXED assuming an unstructured covariance structure: model = baseline, treatment, visit, and treatment-by-visit interaction, and baseline-by-visit interaction. All assessments were set to missing from the time a patient discontinued study medication early. No imputation was used for missing HAQ-DI scores.

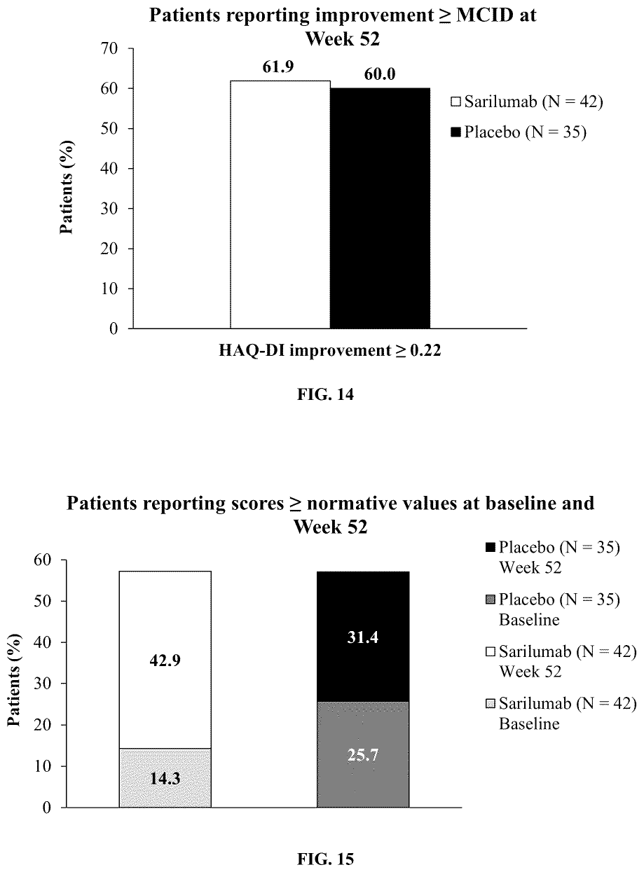

The percent of patients treated with sarilumab and placebo that reported an improvement greater than or equal to MCID for HAQ-DI score at week 52 is shown in FIG. 14. MCID for HAQ-DI was an improvement greater than or equal to 0.22. These results show that sarilumab treatment led to numerically greater improvements in HAQ-DI score than treatment with placebo (with a lower HAQ-DI score representing an improvement).

The percent of patients treated with sarilumab and placebo that reported HAQ-DI scores greater than or equal to normative values at baseline and at week 52 is shown in FIG. 15. The threshold for normative values was less than or equal to 0.25. These results show that sarilumab treatment led to numerically greater improvements in HAQ-DI score than treatment with placebo.

### Patient Global Assessment of Disease Activity (PtGA)

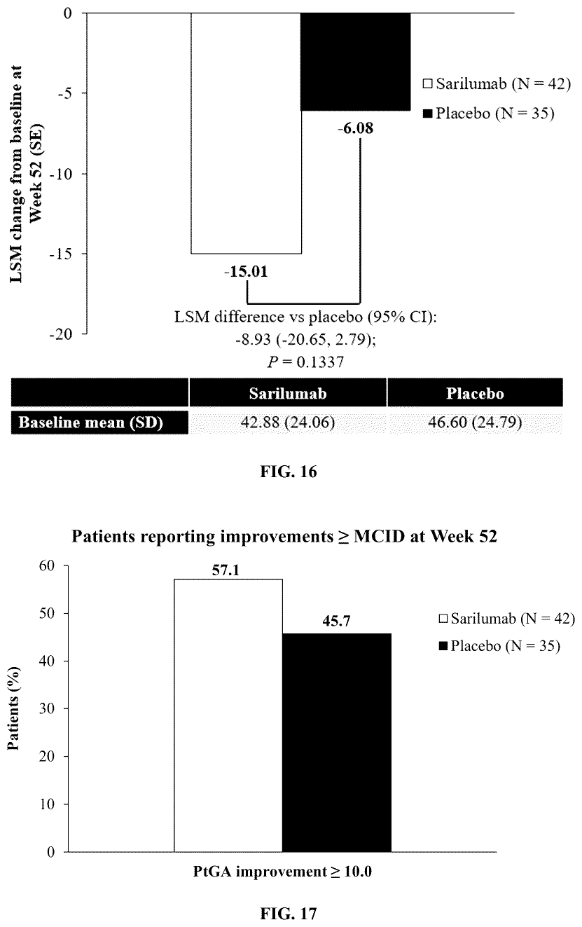

The LSM change from baseline at week 52 for Patient Global Assessment of Disease Activity (PtGA) score for patients treated with sarilumab and placebo is shown in FIG. 16. These results show that treatment with sarilumab led to numerically greater improvements in PtGA score versus treatment with placebo (with a higher score representing a higher level of disease activity or a worse global health).

The percent of patients reporting improvements greater than MCID at week 52 in PtGA score with treatment with sarilumab and placebo is shown in FIG. 17. MCID was an improvement of greater than or equal to 10.0. These results show that treatment with sarilumab led to numerically greater improvements in PtGA scores versus treatment with placebo.

### Pain Visual Analog Scale (VAS)

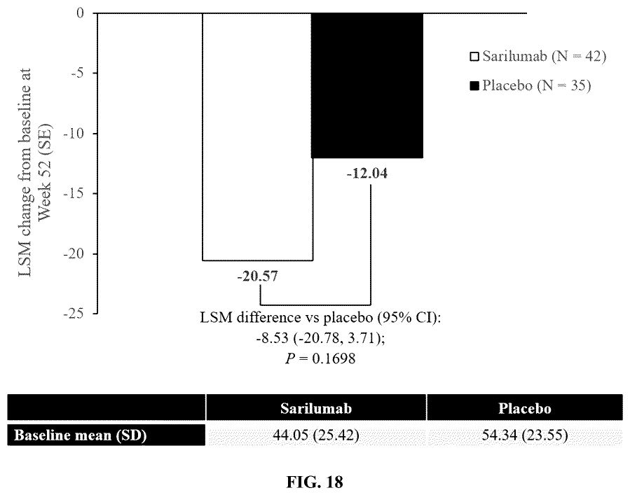

The LSM change from baseline at week 52 for Pain Visual Analog Scale (VAS) score for patients treated with sarilumab and placebo is shown in FIG. 18. These results show that treatment with sarilumab led to numerically greater improvements in Pain VAS scores versus treatment with placebo (with a higher score representing greater pain intensity).

### Resolution of PMR Signs and Symptoms

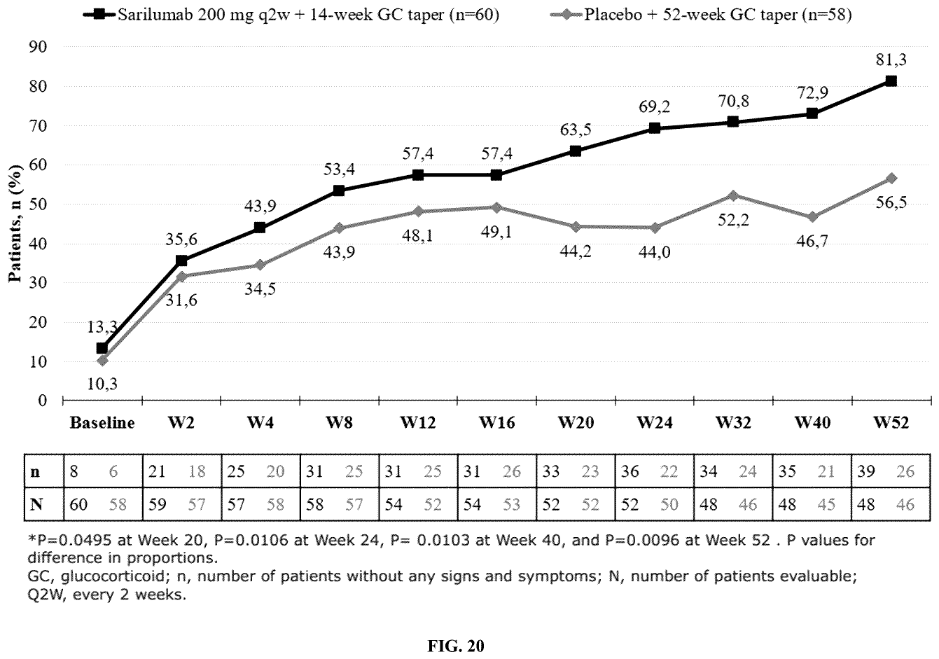

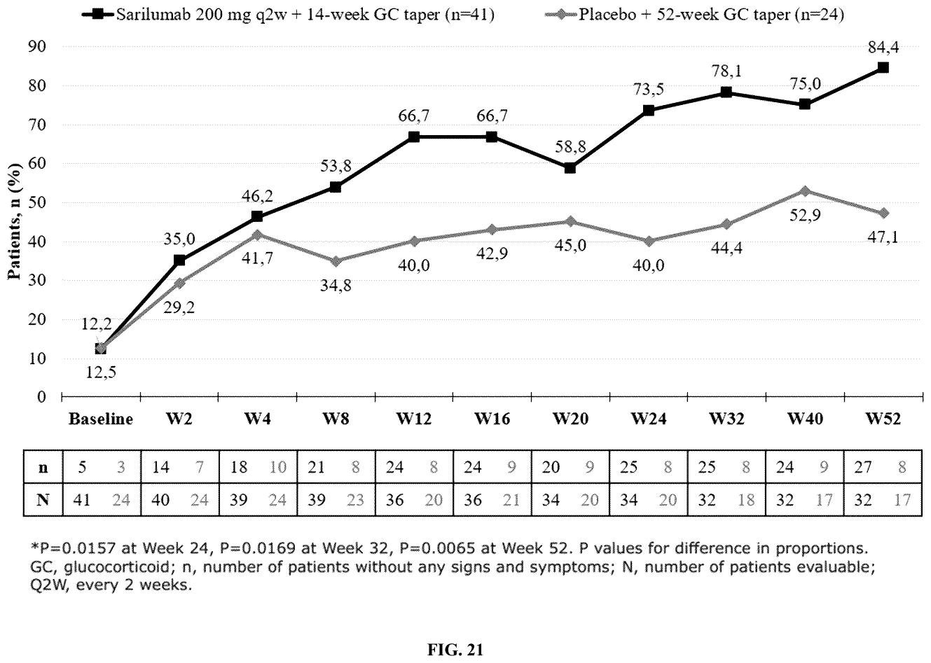

The proportion of patients without any PMR signs and symptoms per visit with sarilumab (200 mg Q2W+14-week GC taper) treatment and placebo (52-week GC treatment) is shown in FIG. 20. With sarilumab treatment, the proportion of patients without PMR signs and symptoms increased versus placebo at week 2 and continued to increase over time until week 52. At each visit after baseline, the proportion of patients without any PMR signs and symptoms was higher in the sarilumab treatment group. Additionally, a greater difference between the two treatment groups was observed in FIG. 21 than in FIG. 20, because patients receiving rescue therapy were excluded from the analysis in FIG. 21.

### Rescue Therapy

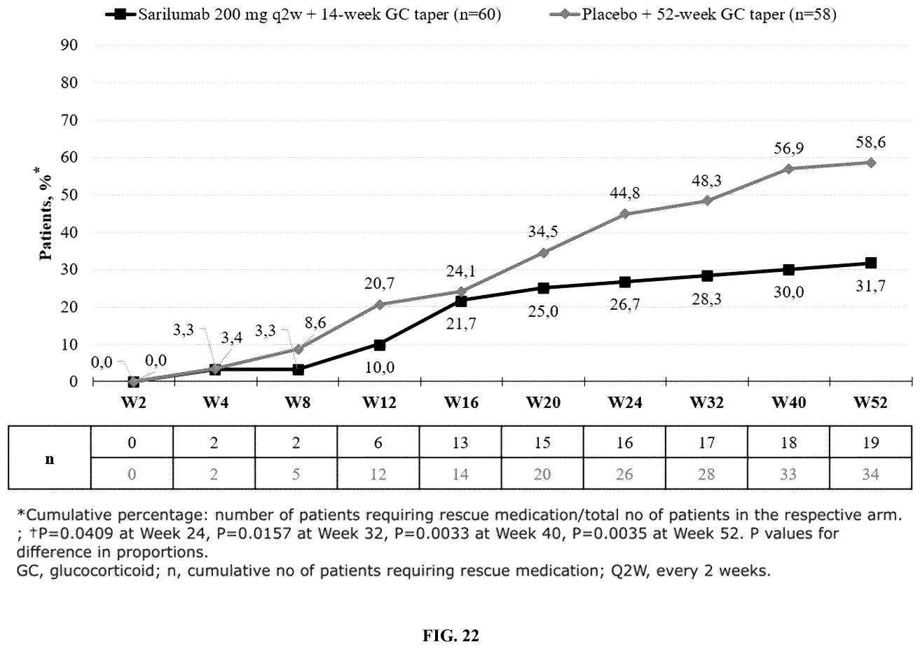

The cumulative proportion of patients who received rescue therapy with sarilumab (200 mg Q2W+14-week GC taper) treatment and placebo (52-week GC treatment) is shown in FIG. 22. These results show that the cumulative proportion of patients requiring rescue therapy was higher in the placebo treatment group at each timepoint after baseline up to week 52.

A higher proportion of patients in the placebo treatment group, versus the sarilumab treatment group, required additional GCs during the study period (58.6% versus 32.2%; P=0.0053 [Fisher's exact test]). In a subgroup of patients that required rescue GC, the median cumulative rescue GC dose over 52 weeks was lower in the sarilumab arm (1076.1 mg [range, 8-2108]) than in the placebo treatment group (1326.5 mg [range, 20-2484]; P=0.5078).

### Pharmacodynamics, Pharmacokinetics and Anti-Drug Antibodies

### Pharmacokinetic Results

After multiple SC administrations of sarilumab 200 mg q2w, the observed trough concentrations of functional sarilumab increased over time and reached steady-state by week 24 with an accumulation ratio of approximately 5-fold.

### Pharmacodynamic Results

After subcutaneous administration of sarilumab, mean serum IL-6 concentrations increased to peak levels at week 12 for the sarilumab 200 mg q2w+14-week taper group and then declined after further treatment. Mean serum total sIL-6Rα concentrations increased rapidly by week 2 and reached steady state at week 24.

At week 52, the mean CRP levels decreased from baseline by 6.9 mg/dL in the sarilumab 200 mg q2w+14-week taper group versus 1.7 mg/dL in the placebo+52-week taper group. This decrease was greater in the sarilumab 200 mg q2w+14-week taper group at all time points versus the placebo+52-week taper group.

### Immunogenicity

One participant in the placebo+52-week taper group and 2 participants in the sarilumab 200 mg q2w+14-week group had a treatment-emergent positive ADA response.

### Adverse Events

### Safety Results

The overall incidence of SAE was low in both the treatment groups (sarilumab 200 mg+14-week taper group: 8 [13.6%] participants; placebo+52-week taper group: 12 [20.7%] participants). The most frequently reported SAEs occurred in the infections and infestations SOC (3 [5.1%] participants in the sarilumab 200 mg+14-week taper group and 3 [5.2%] participants in the placebo+52-week taper group).

The overall incidence of Teas leading to permanent treatment discontinuation was low in both the treatment groups (sarilumab 200 mg q2w+14-week taper group: 7 [11.9%] participants; placebo+52-week taper group: 4 [6.9%] participants). The most frequently reported TEAEs leading to treatment discontinuation occurred in the SOCs of infections and infestations (COVID-19, intervertebral discitis, and pneumonia, each in 1 [1.7%] participant) and blood and lymphatic system disorders (neutropenia in 3 [5.1%] participants).

Overall, the mean changes in clinical laboratory values and vital signs were similar across the study treatment groups with the exception of neutropenia, as higher incidence was observed in sarilumab 200 mg q2w+14-week taper group.

### Conclusions

Enrollment in Study EFC15160 was discontinued by the Sponsor in July 2020 due to a combination of protracted recruitment timelines and the impact of the COVID-19 pandemic on enrollment. The study enrolled 118 (117 treated) participants of the intended 280 participants due to the premature termination of the study. All the enrolled participants were allowed to complete the study as originally planned. As a result of enrolling less than originally planned participants, a protocol amendment was implemented that changed the Type I error probability from <0.01 to <0.05 prior to database lock.

Overall, the results of this study demonstrated that treatment with sarilumab 200 mg q2w+14-week taper in participants with PMR provided greater benefit than treatment with a 52-week CS taper alone. Despite the accelerated corticosteroid taper in sarilumab treated participants, a significantly greater proportion of participants were able to achieve sustained remission at week 52 (10.3% participants in placebo+52-week taper group versus 28.3% participants in sarilumab 200 mg q2w+14-week taper group; p=0.0193). This effect was consistent across pre-specified subgroups of age, sex, race, region, and BMI which all favored sarilumab 200 mg q2w+14-week taper group. To further ascertain the robustness of this effect, a pre-specified sensitivity analysis was performed removing acute phase reactants from the definition of sustained remission, and in this analysis sarilumab treated participants had a consistent and statistically significant increase in the proportion of participants achieving complete remission at week 52 (13.8% for placebo+52-week taper group versus 31.7% for sarilumab 200 mg q2w+14-week taper group; p=0.0280).

An important secondary endpoint aim was to reduce the cumulative steroid usage in sarilumab treated PMR participants with known vulnerability to unwanted side effects of corticosteroid treatments, and in this study sarilumab treated participants had significantly less steroid use versus placebo comparing median values (2044.0 for placebo+52-week taper group versus 777.0 mg sarilumab 200 mg q2w+14-week taper group; p<0.0001). Furthermore, the median of the difference between the actual and expected cumulative CS dose was significantly lower in the sarilumab group compared to the placebo group (median: 0.0 mg versus 199.5 mg; p=0.0189). This represents a clinically significant difference, and when comparing the actual versus expected steroid use in this study, a favorable reduction in steroid use was observed for sarilumab driven by the increased flare rate in the placebo+52-week taper group.

The majority of secondary endpoints were achieved. A greater proportion of participants in the sarilumab 200 mg q2w+14-week taper group achieved disease remission by week 12 (46.7% versus 37.9% in sarilumab 200 mg q2w+14-week taper group and placebo+52-week taper group, respectively). Additionally, more sarilumab treated participants had an absence of PMR flares after clinical remission compared with placebo (55.0% versus 32.8%). The time to first PMR flare was statistically significantly longer in sarilumab treated participants where sarilumab treated participants never reached the median. Finally, a higher adherence to the prednisone taper was observed for sarilumab+14-week taper group compared to the placebo+52-weeks taper group.

The number of participants without any PMR signs and symptoms (excluding participants who had rescue therapy) was higher at each visit in the sarilumab 200 mg q2w+14-week taper group.

Participants in the sarilumab 200 mg q2w+14-week taper group had a 56% less risk of a PMR flare after achieving clinical remission.

Participants in the sarilumab 200 mg q2w+14-week taper group demonstrated a numerical trend for decreased steroid toxicity in GTI, but did not achieve statistical significance.

Exploratory efficacy endpoints demonstrated favorable functional and symptomatic benefit from sarilumab treatment in combination with rapid 14-week corticosteroid taper versus 52-week corticosteroid taper in placebo.

Change from baseline in PMR activity score at week 52 favored sarilumab with a LS mean difference of −5.302 (p=0.0002).

Change from baseline in MD-VAS score at week 52 favored sarilumab with a LS mean difference −10.097 (p=0.0411).

Change from baseline in FACIT-Fatigue score at week 52 showed a favorable trend for sarilumab with a LS mean difference of 3.740 (p=0.0567).

Change from baseline in EQ-5D score at week 52 favored sarilumab with a LS mean difference of 0.130 (p=0.0336).

Change from baseline in EQ5D-VAS score at week 52 showed a favorable trend for sarilumab with a LS mean difference of 8.830 (p=0.0835).

Change from baseline in SF-36 physical component score at week 52 favored sarilumab with a LS mean difference of 4.487 (p=0.0172).

Change from baseline in SF-36 mental component score at week 52 favored sarilumab with a LS mean difference of 4.748 (p=0.0295).

Change from baseline in HAQ-DI score at week 52 showed a favorable trend for sarilumab with a LS mean difference of −0.246 (p=0.0543).

This study demonstrated that the use of sarilumab 200 mg administered every 2 weeks in conjunction with a rapid 14-week taper of corticosteroids in PMR participants resulted in a clinically relevant and statistically significant difference from placebo+52-week CS taper for the primary, majority of secondary, and many of the exploratory endpoints.

In this study, sarilumab demonstrated efficacy in patients with GC-resistant PMR. A greater proportion of patients in the sarilumab treatment arm had no PMR signs and symptoms at each timepoint after baseline versus the placebo arm, confirming that sarilumab provided faster and sustained relief of PMR activity versus GC alone. The requirement for rescue GC therapy was lower in sarilumab recipients than in the placebo arm, despite the fact that sarilumab recipients received less GC than the placebo arm.

The use of sarilumab 200 mg q2w in conjunction with a rapid 14-week taper of CS in PMR revealed no new safety signals and was consistent with the known safety profile of sarilumab. No deaths were reported. The safety observations from this study, when taken together with the observed clinical benefit supports a favorable risk benefit and clinical utility of sarilumab for the treatment of PMR.

Sarilumab is for the treatment of polymyalgia rheumatica (PMR) in adult patients who have had an inadequate response to corticosteroids or who cannot tolerate corticosteroid taper.

### Example 2. Glucocorticoid (GC)-Free Resolution of Polymyalgia Rheumatica (PMR) Signs and Symptoms in Patients Treated with Sarilumab with History of Flare: Analysis from SAPHYR (NCT03600818, EFC15160, Phase 3)

In the SAPHYR study (NCT03600818), a significantly greater proportion of patients receiving sarilumab+glucocorticoid (GC) taper achieved sustained remission at week (W) 52 vs. placebo+GC taper in patients with polymyalgia rheumatica (PMR). Post-hoc data on the resolution of PMR signs and symptoms (S&S) and GC-free resolution of PMR S&S is shown over time.

### Methods

Patients, recruited between 10/2018 and 07/2020, received sarilumab 200 mg Q2W+14-W GC taper (sarilumab arm) OR placebo Q2W+52-W GC taper (comparator arm) for 52W. All patients received prednisone 15 mg/day for 2W, followed by blinded prednisone taper. Patients, who experienced disease flare or could not adhere to pre-protocol GC taper received rescue open-label GC.

### Results

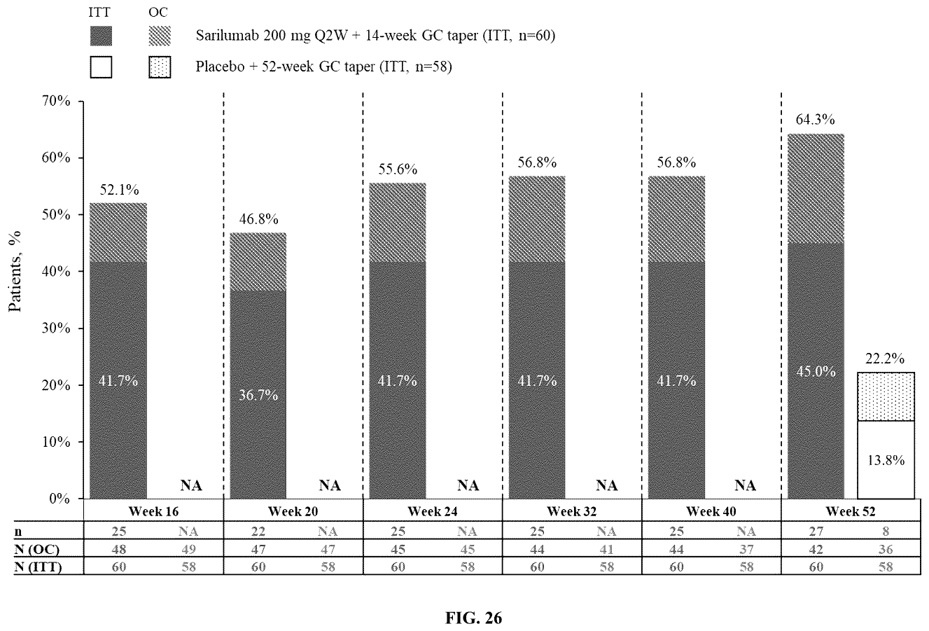

A total of 118 patients were enrolled (sarilumab, n=60 [1 not treated]; comparator, n=58). At each visit after baseline, the proportion of patients without any PMR signs and symptoms was higher in sarilumab vs comparator arms (W52, observed cases [OC]: 81.3% vs. 56.5%; intent-to-treat [ITT]: 65% vs. 45.8%). At W52 for OC and ITT, 64.3% and 45% in sarilumab vs. 22.2% and 13.8% in comparator arms had GC-free resolution of PMR signs and symptoms, respectively (FIG. 26). A lower proportion of patients in sarilumab vs comparator arms required rescue GCs at W52 (31.7% vs. 58.6%).

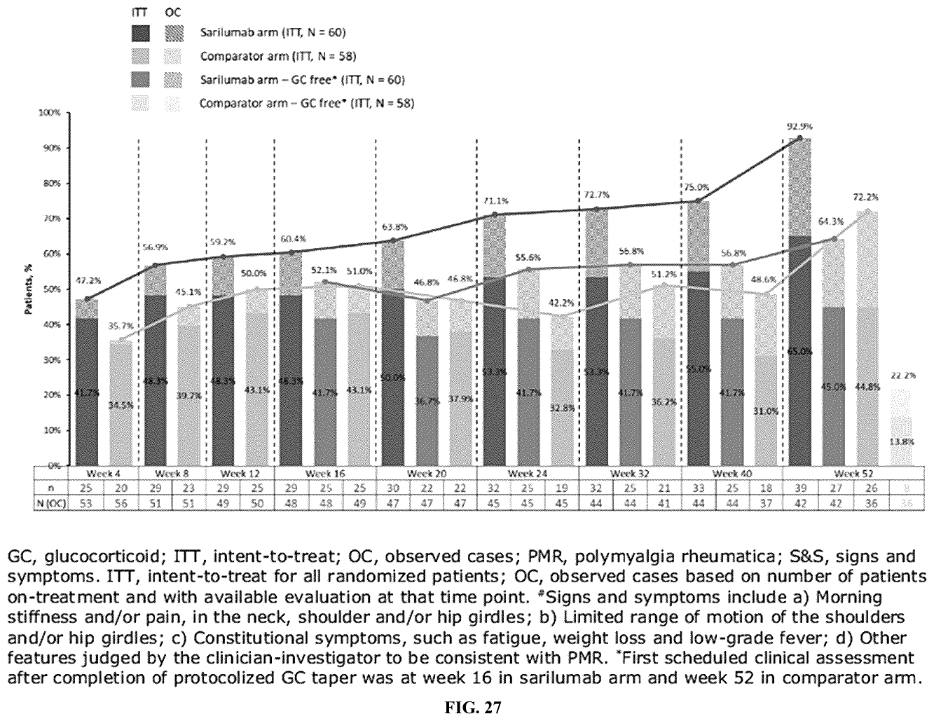

From W4 to W52, the proportion of patients without any PMR signs and symptoms was higher in the sarilumab arm vs. the comparator arm (OC, 92.9% vs 72.2%; ITT, 65% vs. 44.8% at week 52 (FIG. 27).

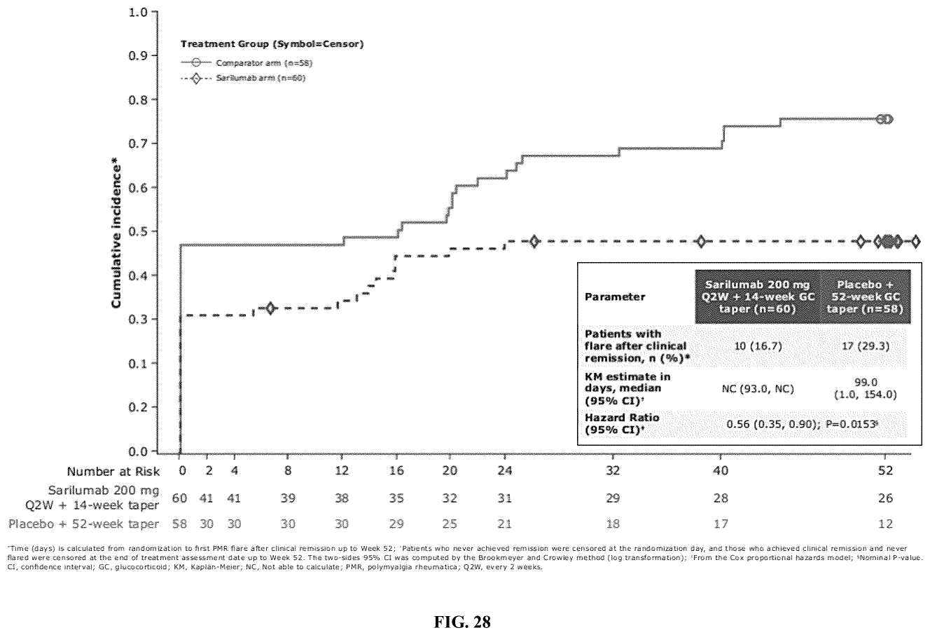

Patients in the sarilumab arm were less likely to have a flare after achieving clinical remission vs the comparator arm (16% vs 29.3%; HR 0.56; 95% CI 0.35-090; P=0.0158). In the sarilumab arm, few flares occurred after W16 while in the comparator arm, flares continued to occur through W52 (FIG. 28).

### Conclusion

The addition of sarilumab to a short 14-W GC taper resulted in rapid resolution of PMR signs and symptoms from W4 compared to a 52-W GC taper. GC-free resolution of PMR signs and symptoms was maintained from W16 to W52 in patients treated with sarilumab.

### Example 3. Effectiveness of Interleukin-6 Receptor Inhibitors for Polymyalgia Rheumatica

### Study Overview

Interleukin-6 receptor (IL-6R) inhibition has been shown to be effective in giant cell arteritis but data are limited in polymyalgia rheumatica (PMR). A retrospective study was conducted to evaluate the effectiveness of IL-6R inhibitors (IL-6Ri; sarilumab or tocilizumab) in 3rd-line and 2nd-line treatment of glucocorticoid (GC)-refractory PMR patients compared to conventional immunomodulatory (cIM) therapy.

### Material and Methods

### Study Objective

To evaluate the treatment effectiveness of IL-6R inhibitor therapy (IL-6Ri) (tocilizumab, sarilumab) in 3rd-line and 2nd-line treatment of GC-refractory PMR compared to conventional disease-modifying antirheumatic drug (cDMARD) therapy (methotrexate, azathioprine, leflunomide).

### Study Design

The study used an observational retrospective comparative cohort design. Adults with PMR, defined as 1 inpatient or 2 outpatient claims with a PMR diagnosis≥30 days apart, were identified from fee for service Medicare medical and Part D prescription claims from Mar. 29, 2016 to Jun. 30, 2020. Included were those who initiated IL-6Ri or cIM as 2nd-line and 3rd-line therapy for PMR and had continuous enrollment for 180 days prior to therapy initiation (baseline). For 3rd-line cohort, the index date was date of switch from cIM to IL-6Ri for the IL-6Ri group and switch from one cIM to another cIM for the cIM group. For 2nd-line cohort, the index date was the date of initiation of IL-6Ri with no prior cIM for the IL-6Ri group and new use of cIM for the cIM group. A 180-day washout period for the index drug was applied to all cohorts. Patients were on GC on index date (≤25 mg/day) and excluded if they had evidence of seropositive rheumatoid arthritis, other inflammatory arthritis or connective tissue disease, multiple sclerosis, or malignancy. Patients on IL-6Ri and cIM were matched 1:1 on age, sex, and GC dose category, and propensity score matching was used to adjust for potential confounders.

Outcomes that were assessed using an adjusted Cox proportional hazards models included: GC discontinuation; GC discontinuation or low dose GC (<2.5 mg/day); and non-persistence (discontinuation of IL-6Ri or cIM, or switch) in the 2nd-line cohort, adjusting for residually imbalanced factors after matching. Censoring occurred at 1 year, death, 60 days before end of enrollment (gap≤30 days), or addition of comparator PMR therapy with additional censoring applied in sensitivity analysis for drug discontinuation and/or switching.

### Inclusion Criteria

### 2nd Line and 3rd Line

The presence of one inpatient or the first of 2 outpatient PMR diagnoses in all prior data.

Oral GC use on index date.

Continuous enrollment from 180 days prior to through 1 day after index date (baseline period).

### 3rd Line Only

Occurrence of cDMARD therapy during baseline prior to index date.

Occurrence of cDMARD therapy on the index date.

### Exclusion Criteria

### 2nd Line and 3rd Line

Age<18 on index date.

Initiated both IL-6Ri and cDMARD therapy on the index date (Simultaneous use of multiple drugs within cDMARD drug groups will be allowed, but >1 cannot be initiated on the same day. Likewise, prior cDMARD use and new IL-6Ri use is permitted, but both cannot be initiated on the same day).

GC dose of >25 mg prednisone equivalents on index date.

Seropositive rheumatoid arthritis.

Diagnosis of other inflammatory arthritis, connective tissue disease, multiple sclerosis, organ transplantation.

Active treatment for malignancy.

### 2nd Line Only

Exclude patients with prior use of either IL-6Ri or cDMARD on available record (at least 180 days).

### Outcomes

### Primary

Discontinuation of oral GC therapy, defined as a gap in oral GC drug supply of >60 days.

Discontinuation of oral GC therapy or achievement of minimal oral GC daily dose (≤2.5 mg prednisone equivalents/day).

### Secondary

Cumulative oral GC dose over follow-up, assessed as cumulative sum and as mg prednisone equivalents per patient per week of contributed follow-up time.

Time to discontinuation of index therapy (gap >60 days) (2nd-line cohort only).

### Results

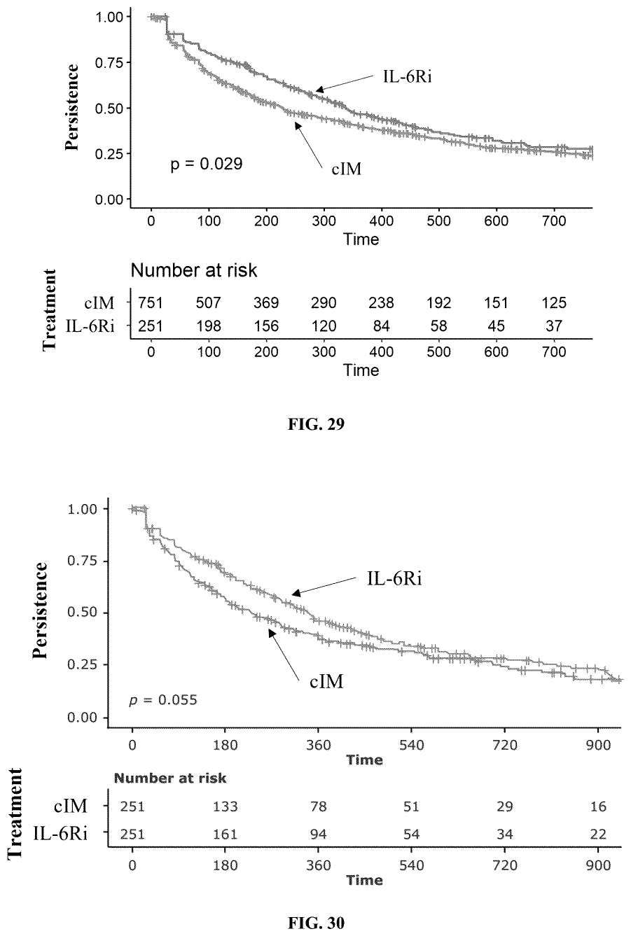

After matching, 409 3rd-line and 251 2nd-line patients were included in the analysis. As shown in Table 31, post-match, patient characteristics were generally well balanced between cohorts, with small differences between exposure groups. Median time from first PMR diagnosis to start of follow-up was approximately 1.75 years (3rd-line) and 0.90 years (2nd-line) and similar between groups. As shown in Table 32, despite matching on GC category, IL-6Ri patients started on higher doses of GC. As shown in Table 33, after matching and adjustment, patients receiving IL-6Ri were more likely to achieve GC discontinuation or low dose GC, compared to cIM patients. The unadjusted outcomes are shown in Table 34 for 2nd Line and Table 35 for 3rd Line. As shown in FIG. 29, time to discontinuation of IL-6Ri or cIM, or switch (non-persistence) was significantly greater for IL-6Ri vs. cIM patients (p=0.029).

TABLE 31
Post-match characteristics of PMR patients,
stratified by 2nd and 3rd line therapy use

| | IL-6Ri | cIM | SMD* |
|---|---|---|---|
| Third Line Therapy | | | |
| Patient count, n | 409 | 409 | |
| Age, mean (SD) | 73.9 (6.1) | 73.7 (6.0) | 0.035 |
| Female Sex, % | 70.2 | 73.1 | 0.065 |
| White Race, % | 93.4 | 93.9 | 0.028 |
| Charlson Score, mean (SD) | 2.5 (2.0) | 2.5 (1.8) | 0.015 |
| Disabled, % | 13.7 | 16.4 | 0.075 |
| Seronegative RA, % | 63.1 | 72.1 | 0.194 |
| Giant cell arteritis (w/o PMR) | 29.1 | 15.6 | 0.327 |
| Outpatient office visits, mean (SD) | 10.6 (5.4) | 10.2 (5.6) | 0.072 |
| Second Line Therapy | | | |
| Patient count, n | 251 | 251 | |
| Age, mean (SD) | 75.8 (6.6) | 78.0 (6.1) | 0.339 |
| Female Sex, % | 26.7 | 27.1 | 0.009 |
| White Race, % | 86.1 | 87.6 | 0.138 |
| Charlson Score, mean (SD) | 2.5 (2.0) | 2.6 (1.9) | 0.049 |
| Disabled, % | 9.6 | 12.0 | 0.077 |
| Seronegative RA, % | 30.3 | 25.9 | 0.098 |
| Giant cell arteritis (w/o PMR) | 18.7 | 41.4 | 0.511 |
| Outpatient office visits, mean (SD) | 10.8 (6.4) | 10.6 (5.4) | 0.028 |

*SMDs >0.10 are considered potentially clinically meaningful; factors residually imbalanced (bold) were adjusted for in the outcome models
cIM: conventional immunomodulatory;
IL-6Ri: interleukin-6 receptor inhibitor;
PMR: polymyalgia rheumatica;
RA: rheumatoid arthritis;
SMD: standardized mean difference

TABLE 32
Weekly GC dose over time 3rd line therapy

| | Index | N | | | | | |
|---|---|---|---|---|---|---|---|
| Period | Therapy | Obs | Mean | Std Dev | 25th Pctl | 50th Pctl | 75th Pctl |
| 1-90 | CDMARD | 409 | 50.7 | 32.6 | 31.6 | 43.6 | 65.0 |
| | IL6 | 409 | 63.4 | 42.9 | 33.8 | 56.9 | 82.8 |
| 91-180 | CDMARD | 408 | 42.9 | 32.8 | 23.2 | 34.2 | 54.3 |
| | IL6 | 408 | 47.9 | 41.0 | 19.8 | 39.0 | 64.6 |
| 181-270 | CDMARD | 398 | 45.1 | 35.9 | 21.8 | 34.6 | 58.3 |
| | IL6 | 403 | 47.5 | 47.7 | 18.3 | 34.2 | 60.7 |
| 271-365 | CDMARD | 370 | 40.6 | 30.4 | 21.4 | 33.9 | 53.4 |
| | IL6 | 379 | 44.4 | 42.5 | 17.8 | 33.3 | 57.0 |

TABLE 33
Outcomes associated with IL-6Ri compared to cIM therapy

| | | Adjusted** |
|---|---|---|
| | | Hazard Ratio |
| Outcome | Additional* Censoring | (95% CI) |
| Stop GC | Stopping index drug, switching, 1 year | 1.27 (1.05-1.53) |
| Stop or | Stopping index drug, switching, 1 year | 1.22 (1.03-1.45) |
| low dose GC | | |
| Stop GC | Stopping index drug, switching | 1.17 (0.99-1.40) |
| Stop GC | None | 1.26 (1.08-1.47) |
| Stop GC | Switching, 1 year | 1.33 (1.13-1.56) |

*All models censored for outcome, death, or end of the study period (Dec. 31, 2020) After matching, and adjusted for any factors residually imbalanced with SMD >0.10, stratified by 2^{nd} or 3^{rd} line therapy (age, race, calendar year, seronegative RA, and giant cell arteritis)
CI = Confidence Interval;
GC = Glucocorticoid;
SMD = standardized mean difference

TABLE 34
Unadjusted Outcomes associated with IL-
6Ri compared to cIM therapy for 2^{nd} Line

| Outcome | cDMARD | | IL-6Ri | | P-value |
|---|---|---|---|---|---|
| Patients with glucocorticoid | 80 | (32%) | 123 | (49%) | <0.001 |
| discontinuation, n (%) | | | | | |
| Patients with glucocorticoid | 97 | (39%) | 139 | (55%) | <0.001 |
| discontinuation or minimal | | | | | |
| dose, n (%) | | | | | |
| Patients with glucocorticoid | 92 | (37%) | 135 | (54%) | <0.001 |
| discontinuation at (No censor | | | | | |
| one year), n (%) | | | | | |
| Cumulative GC dose (mg | | | | | 0.9 |
| prednisoneequivalents per | | | | | |
| person-week) | | | | | |
| Mean (SD) | 49 | (34) | 51 | (40) | |
| Median (IQR) | 40 | (27, 63) | 40 | (21, 69) | |

TABLE 35
Unadjusted Outcomes associated with IL-
6Ri compared to cIM therapy for 3rd Line

| Outcome | cDMARD | | IL-6Ri | | P-value |
|---|---|---|---|---|---|
| Patients with glucocorticoid | 123 | (30%) | 184 | (45%) | <0.001 |
| discontinuation, n (%) | | | | | |
| Patients with glucocorticoid | 144 | (35%) | 203 | (50%) | <0.001 |
| discontinuation or minimal | | | | | |
| dose, n (%) | | | | | |
| Patients with glucocorticoid | 145 | (35%) | 193 | (47%) | <0.001 |
| discontinuation at (No censor | | | | | |
| one year), n (%) | | | | | |
| Cumulative GC dose (mg | | | | | 0.9 |
| prednisoneequivalents per | | | | | |
| person-week) | | | | | |
| Mean (SD) | 48 | (32) | 50 | (40) | |
| Median (IQR) | 39 | (27, 63) | 41 | (25, 67) | |

### Conclusions

IL-6Ri therapy was more effective as a steroid sparing agent compared to cIM therapy for adult patients with PMR.

### Example 4. Effectiveness of Interleukin-6 Receptor Inhibitors for Polymyalgia Rheumatica

### Study Objective

To evaluate the treatment effectiveness of IL-6R inhibitor therapy (IL-6Ri) (tocilizumab, sarilumab) as 3rd-line (3L) and 2nd-line (2 L) treatment options for GC-refractory PMR compared with that of cIM therapy (methotrexate, azathioprine, leflunomide).

### Material and Methods

### Study Design

This retrospective observational cohort study included adult patients (≥50 years) with PMR identified from national fee-for-service Medicare medical and Part D prescription claims data from Jan. 1, 2006 to Dec. 31, 2020.

Patients who initiated IL-6Ri or cIM as 2 L and 3 L therapy (proxy for GC-refractory) for PMR and had continuous enrollment for ≥180 days prior to therapy initiation (baseline) were included as 2 separate cohorts.

The 3 L cohort compared patients who previously received cIM and initiated a new IL-6Ri therapy (exposed) versus patients who initiated a new cIM therapy different from the prior CIM (referent) for treatment of PMR.

For the 3 L cohort, the index date was the date of initiation of an IL-6Ri therapy after a prior cIM therapy or a cIM therapy different from a prior cIM therapy preceded by no IL-6Ri, using all available data.

The 2 L cohort compared patients with PMR receiving a new IL-6Ri therapy (exposed) versus new cIM therapy (referent), with no prior use of either IL-6Ri or CIM therapy. For the 2 L cohort, the index date was the date of initiation of first IL-6Ri or CIM preceded by no IL-6Ri or cIM, using all available data.

Patients initiating IL-6Ri and a new CIM initially were matched 1:3 on age, sex, and GC dose category (in the 3 L cohort, recency of prior CIM therapy) and then matched 1:1 on the propensity score (PS) to control for potential confounders.

### Inclusion & Exclusion Criteria

Patients with 1 inpatient or 2 outpatient claims with PMR diagnoses at least 30 days apart from any clinical specialty or claim position, oral GC use on index, and continuous enrollment from 180 days prior to 1 day after index (baseline period) were included in this study. Patients with occurrence of CIM therapy at any time prior to index were eligible for the 3 L cohort only.

Patients were excluded if they had evidence of seropositive rheumatoid arthritis (RA), adult-onset Still's disease, other arthritis or connective tissue disease, organ transplant, or active treatment for malignancy.

### Endpoints

The primary effectiveness endpoints were discontinuation of oral GC therapy and a composite endpoint of GC discontinuation or minimal dose GC (<2 mg/day).

Secondary endpoints included cumulative GC dose received over follow-up, and in the 2 L comparative cohort only, persistence on index PMR therapy (i.e., either IL-6Ri or cIM therapy).

### Statistical Analysis

Comparison of the time-to-GC discontinuation and time to reach the composite endpoint (GC discontinuation or minimal GC dose) were performed in each comparative cohort using the Cox proportional hazard models.

The Cox proportional hazard models were used to estimate hazard ratios (HRs) with 95% confidence intervals (CIs) comparing the IL-6Ri and the CIM-exposure groups, and a pooled HR was estimated, stratifying the separate HRs from the 3 L and 2 L cohorts.

Models were further adjusted for any covariates for which balance was not achieved through PS matching based on a post-match standardized mean difference (SMD) of >0.10.

Three sensitivity analyses were conducted that applied various censoring rules for discontinuation of the index therapy, treatment switching, and at 12 months.

### Results

### Patient Characteristics

After direct matching and PS matching, 409 and 251 patients were included in each treatment arm of the 3 L and 2 L cohorts, respectively.

Patient characteristics were generally well balanced, with a few differences between exposure groups. Among the 3 L cohort, IL-6Ri patients were slightly more likely to be in higher GC dose category at index, less likely to have claims with codes for seronegative RA, and more likely to have claims with codes for concomitant giant cell arteritis (GCA) as compared with CIM patients (Table 36). All other characteristics examined were well balanced (data not shown). Among the 2 L cohort, IL-6Ri patients were slightly younger, less likely to be enrolled in Medicare due to disability, and more likely to have claims with codes for seronegative RA and GCA (Table 36).

The median time from the first PMR diagnosis observed in claims to the start of follow-up was approximately 1.75 years (3 L cohort) and 0.90 years (2 L cohort) and similar between groups.

TABLE 36
Post-math and PS matched characteristics of PMR patients,
stratified by third-line and second-line therapy use

| 3L Cohort | IL-6Ri (n = 409) | | CIM (n = 409) | | SMD^{a} |
|---|---|---|---|---|---|
| Age, mean (SD) | 73.9 (6.1) years | | 73.6 (5.9) years | | 0.04 |
| Female, n (%) | 287 | (70.2%) | 286 | (69.9%) | 0.01 |
| Original reason eligible | | | | | 0.05 |
| for Medicare, n (%) | | | | | |
| Age 65 or older | 353 | (86.3%) | 346 | (84.6%) | |
| Disabled | 56 | (13.7%) | 63 | (15.4%) | |
| Charlson comorbidity index | 2.5 | (2.0) | 2.5 | (1.8) | 0.00 |
| score, mean (SD) | | | | | |
| Index day prednisone- | | | | | 0.11 |
| equivalent dose, n (%) | | | | | |
| <5 | 50 | (12.2%) | 38 | (9.3%) | |
| 5-10 | 116 | (28.4%) | 131 | (32.0%) | |
| 10+ | 243 | (59.4%) | 240 | (58.7%) | |
| Seronegative RA, n (%) | 258 | (63.1%) | 285 | (69.7%) | 0.14 |
| GCA without PMR, n (%) | 119 | (29.1%) | 61 | (14.9%) | 0.35 |
| 2L Cohort | IL-6Ri (n = 251) | | CIM (n = 251) | | SMD^{a} |
| Age, mean (SD) | 75.8 | (6.6) | 77.8 | (6.2) | 0.31 |
| Female, n (%) | 184 | (73.3%) | 182 | (72.5%) | 0.02 |
| Original reason eligible | | | | | 0.11 |
| for Medicare, n (%) | | | | | |
| Age 65 or older | 227 | (90.4%) | 218 | (86.9%) | |
| Disabled | 24 | (9.6%) | 33 | (13.1%) | |
| Charison comorbidity index | 2.5 | (2.0) | 2.5 | (1.8) | 0.01 |
| score, mean (SD) | | | | | |
| Index day prednisone- | | | | | 0.07 |
| equivalent dose, n (W) | | | | | |
| <5 | 28 | (11.2%) | 29 | (11.6%) | |
| 5-10 | 62 | (24.7%) | SS | (21.9%) | |
| 10+ | 161 | (64.1%) | 167 | (66.5%) | |
| Seronegative RA, n (%) | 76 | (30.3%) | 60 | (23.9%) | 0.14 |
| GCA without PMR, n (%) | 104 | (41.4%) | 46 | (18.3%) | 0.52 |

CIM, conventional immunomodulators; GCA, giant cell arteritis; IL-6Ri, interleukin-6 receptor inhibitor; PMR, polymyalgia rheumatica; PS, propensity score; RA, rheumatoid arthritis; SMD, standardized mean
difference.
^{a}SMDs >0.10 are considered potentially clinically meaningful; factors residually imbalanced were adjusted in the outcome models. Patients were matched on age (±3 years), gender, recency of prior CIM therapy (1-60 days, 61-180 days, >180 days), and daily GC does category at index (<5 mg, 5-10 mg, >10 mg).

### Glucocorticoid Discontinuation or Minimal Dose Glucocorticoid

Based on the HRs shown in Table 37, patients receiving IL-6Ri were more likely to discontinue GC (HR [95% CI]:1.32 [1.09-1.58]) and achieve minimal or no GC use (1.30 [1.90-1.54]) compared with the cIM group, and results remained statistically significant regardless of the censoring rules applied in sensitivity analysis.

IL-R6i initiators were more likely to discontinue GC use (3 L cohort: 45.0% vs. 30.1% and 2 L cohort: 49.0% vs 32.7%) and discontinue or achieve minimal does of GC (3 L cohort: 49.6% vs. 34.7% and 2 L cohort: 55.4% vs. 39.4%) compared with CIM initiators (average follow-up time for IL-6Ri initiators: 147 days and 163 days for 3 L2 L, respectively; for CIM initiators: 138 days and 148 days for 3 L and 2 L, respectively).

Cumulative prednisone-equivalent dose during follow-up was not significantly different between groups.

Results from sensitivity analyses that varied censoring rules yielded similar results. The separate HRs comparing IL-6Ri with CIM therapy in each of the 3 L and 2 L cohorts were similar.

TABLE 37
Pooled adjusted hazard ratios stratified by third-
line and second-line therapy and varying censoring
rules across several sensitivity analyses.

| | Adjusted HR^{a} |
|---|---|
| Outcome and censoring rules | (95% CI) |
| Primary outcome, main analysis: discontinue | 1.32 |
| GC, censoring for enrollment end - 60 days, | (1.09-1.58) |
| 1 year, death, outcome, stop index drug, | |
| switching | |
| Composite outcome, main analysis: discontinue | 1.30 |
| or minimal GC, censoring for enrollment end - | (1.09-1.54) |
| 60 days, 1 year, death, outcome, stop index | |
| drug, switching | |
| Primary outcome, sensitivity analysis 1: | 1.22 |
| discontinue GC, censoring for enrollment | (1.02-1.45) |
| end - 60 days, death, outcome, stop index | |
| drug, switching | |
| Primary outcome, sensitivity analysis 2: | 1.28 |
| discontinue GC, censoring for enrollment | (1.10-1.50) |
| end - 60 days, 1 year, death, outcome | |
| Primary outcome, sensitivity analysis 3: | 1.36 |
| discontinue GC, censoring for enrollment | (1.16-1.59) |
| end - 60 days, 1 year, death, outcome, | |
| switching to a new CIM | |

^{a}Adjusted for: age, region, original reason for Medicare, baseline weekly prednisone-equivalent dose, COPD, seronegative RA, and GCA. CI, confidence interval; CIM, conventional immunomodulator; COPD, chronic obstructive pulmonary disorder; GC, glucocorticoids; GCA, giant cell arthritis; HR, hazard ratio; RA, rheumatoid arthritis

### Treatment Persistence, Continuation, and Switching

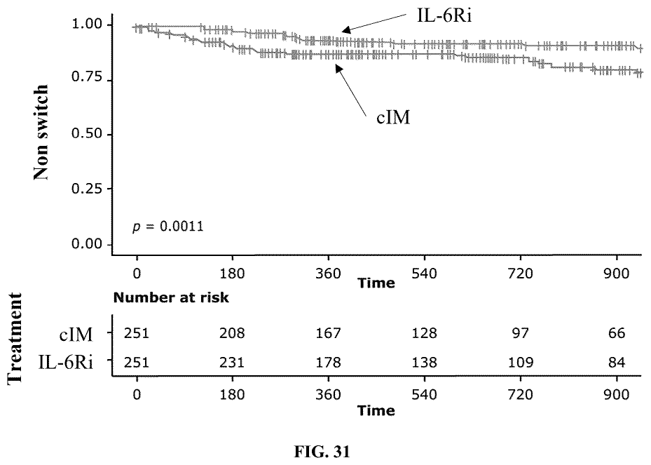

After PS matching, persistence on 2 L treatment trended in the favor of IL-6Ri therapy compared with cIM therapy (p=0.055; FIG. 30). IL-6Ri initiators were less likely to switch therapy compared with cIM therapy (p=0.0011; FIG. 31).

### Conclusions

In older patients with PMR, IL-6Ri therapy was as effective as a steroid-sparing agent compared with cIM therapy when used either as a 3 L or 2 L therapy. These results are consistent with efficacy of IL-6Ri therapy in PMR.

### Example 5. Long Term Safety of Conventional and Biologic IL-6Ri Immunomodulators as Second- or Third-Line Treatment of Polymyalgia Rheumatica

Data are presented on safety of IL-6Ri and conventional immunomodulatory (CIM) therapy for up to 2 years (Y) in patients with PMR.

### Objective

Evaluate safety of IL-6Ri and CIM therapy in second (2 L)- or third-line (3 L) treatment of PMR.

### Methods

This was a retrospective study in US adults ≥50 Y, with 1 inpatient/2 outpatient claims with PMR diagnosis ≥30 days apart, from national fee-for-service Medicare data (Mar. 29, 2016-Jun. 30, 2020). Patients were on ≥25 mg prednisone (PS) equivalent and started IL-6Ri (tocilizumab/sarilumab) or CIM (methotrexate/leflunomide/azathioprine) as 2 L/3 L therapy and had continuous enrollment ≥180 days prior to index. For 2 L, index date was start of IL-6Ri/CIM with no prior use of IL-6Ri/CIM. For 3 L, index date was start of IL-6Ri (with/without background CIM) or new CIM, after prior CIM. Follow-up was until the earliest of end of enrollment, death, treatment switch, or 2Y. Patients with seropositive RA, other arthritis or connective tissue disease, giant cell arteritis, organ transplant, or malignancy undergoing treatment were excluded. Stratifying by line of therapy, IL-6Ri patients were direct matched using 1:3 variable ratio matching on age, sex, index date and baseline PS equivalent dose category (<2.5, 2.5-<5, 5-<10, 15-<20, 20-25 mg), recency of prior CIM (3L only; 1-60, 61-180, 180+days), and then 1:1 propensity score matched using multivariate logistic regression to identify variables with SMD>0.1. We examined hospitalized infection (any and primary diagnosis), gastrointestinal perforation, major adverse cardiac events, malignancy, and drug induced liver injury. Except for infection, patients with prior events were excluded from each outcome-specific analysis. Outcomes were identified using validated or previously used claims-based algorithms. Incidence rates were reported per 100 patient-years through 2Y of follow-up, using exact Poisson for 95% CI, if events <5. Cox proportional hazards model was used to estimate adjusted hazard ratios (aHR) for infections (given paucity of other events). Sensitivity analyses examined events in Y1 and Y2 separately and stratified by 2 L vs. 3 L.

### Results

In final cohort, covariate balance for 451 matched patients (2 L: 183; 3L: 268) in each arm was good. Residual imbalances were small for disability status, steroid dose category, and chronic pulmonary disease. Incidence rates and aHRs for events are shown in Table 38; aHRs were not significant for IL-6Ri vs CIM. Sensitivity analyses were consistent with primary results and generally showed incidence was similar, or numerically lower for IL-6Ri vs CIM in Y2 and lower for CIM vs IL-6Ri in Y1, for most outcomes.

TABLE 38
Pooled incidence rates and aHIR stratified by 2L/3L of safety events
for IL-6Ri and CIM-treated patients with up to 2 Y exposure.

| | Exposure | | | *aHR (95% CI) |
|---|---|---|---|---|
| Outcome | group | Events/100PY | | IL-6Ri vs CIM |
| Hospitalized | IL-6Ri | 14.4 | (11.8-17.7) | 1.05 (0.79-1.41) |
| Infection, any | CIM | 13.4 | (10.8-16.6) | |
| Hospitalized | IL-6Ri | 9.8 | (7.7-12.5) | 1.16 (0.82-1.65) |
| infection, primary | CIM | 8.4 | (6.4-11.0) | |
| diagnosis | | | | |
| Malignancy | IL-6Ri | 0.8 | (0.3-1.9) | NE |
| | CIM | 2.0 | (1.1-3.5) | |
| Major adverse | IL-6Ri | 2.1 | (1.2-3.7) | NE |
| cardiac events | CIM | 2.1 | (1.2-3.7) | |
| Gastrointestinal | IL-6Ri | 0.1 | (0.0-0.8) | NE |
| perforation | CIM | 0.1 | (0.0-0.8) | |
| Drug induced liver | IL-6Ri | 0.0 | (0.0-0.5) | NE |
| injury | CIM | 0.0 | (0.0-0.5) | |

*aHR, adjusted hazard ratios, controlling for covariates imbalanced with SMD >0.10; CI, confidence interval; CIM, conventional immunomodulatory therapy; NE, not estimated, inadequate sample size; PY, patient years; IL-6Ri, interleukin-6 receptor inhibitor

### Conclusions

Rates of serious adverse events for IL-6Ri vs CIM therapy with up to 2Y exposure were comparable in PMR.

1. A method for treating polymyalgia rheumatica (PMR) in a subject in need thereof, comprising administering an effective amount of an antibody or antigen-binding fragment thereof that specifically binds IL-6 receptor.

2. The method of claim 1, wherein the subject has PMR that is refractory to steroids or is refractory to steroid taper or has had an inadequate response to steroids or cannot tolerate steroid taper.

3. (canceled)

4. The method of claim 2, wherein the steroids comprise corticosteroids that optionally comprise prednisone.

5. (canceled)

6. The method of claim 4, wherein the subject was previously treated with a dose of ≥7.5 mg/day, and/or ≤25 mg/day or ≤20 mg/day, of prednisone.

7. The method of claim 1, wherein the antibody or antigen-binding fragment thereof is administered in combination with another therapeutic agent.

8. The method of claim 7, wherein the therapeutic agent comprises:
a corticosteroids;
prednisone; or
prednisone administered at a dose of about 15 mg/day.

9. (canceled)

10. (canceled)

11. The method of claim 4, wherein the dose of corticosteroid is discontinued or tapered or discontinued beginning at between about 10 weeks and about 20 weeks after administering a first dose of the antibody, or discontinued beginning at about 14 weeks after administering a first dose of the antibody, or discontinued or tapered to <2.5 or 2.0 mg prednisone/day.

12. (canceled)

13. The method of claim 1, wherein the subject was previously treated with a disease modifying antirheumatic drug (cDMARD) or concomitantly treated with a cDMARD that is optionally selected from the group consisting of methotrexate, azathioprine, and leflunomide.

14-16. (canceled)

17. The method of claim 1, wherein the antibody or antigen-binding fragment thereof is administered at a dose of between about 150 mg and about 200 mg or about 150 mg or about 200 mg or administered at an initial dose of about 200 mg and one or more secondary doses of about 200 mg administered every other week (q2w).

18-20. (canceled)

21. The method of claim 1, wherein the antibody or antigen-binding fragment thereof comprises: heavy chain complementarity determining region (HCDR) sequences of SEQ ID NOs: 3, 4 and 5, and comprises light chain complementarity determining region (LCDR) sequences of SEQ ID NOs: 6, 7 and 8; or a heavy chain variable region of sequence SEQ ID NO: 1 and the light chain variable region sequence of SEQ ID NO: 2; or a heavy chain comprising SEQ ID NO: 9 and a light chain comprising SEQ ID NO: 10; or the antibody is sarilumab.

22-50. (canceled)

51. A method of reducing or eliminating the dependence of a subject with polymyalgia rheumatica (PMR) on a background therapy comprising corticosteroids for the treatment of PMR comprising:
(a) selecting a subject who has PMR that is partially controlled or uncontrolled with a background therapy comprising corticosteroids;
(b) administering to the patient a defined dose of a therapeutically effective amount of an antibody or antigen-binding fragment thereof that specifically binds IL-6 receptor at a defined frequency for an initial treatment period while maintaining the subject's background PMR therapy for the initial treatment period; and
(c) gradually reducing or eliminating the dosage of corticosteroids administered to the subject over the course of a subsequent treatment period while continuing to administer the antibody or antigen-binding fragment thereof to the subject at the defined frequency and dose used during the initial treatment period.

52. The method of claim 51, wherein:
the corticosteroids comprise prednisone;
the corticosteroids comprising prednisone is about 15 mg/day during the initial treatment period;
the subsequent treatment period is at least 14 weeks;
the subsequent treatment period is at least 52 weeks;
the antibody or antigen-binding fragment thereof is administered at a dose of about 150 mg or 200 mg;
the antibody or antigen-binding fragment thereof is administered at a dose every other week (q2w);
the antibody or antigen-binding fragment thereof comprises heavy chain complementarity determining region (HCDR) sequences of SEQ ID NOs: 3, 4 and 5, and comprises light chain complementarity determining region (LCDR) sequences of SEQ ID NOs: 6, 7 and 8;
the antibody or antigen-binding fragment thereof is administered for at least 12 weeks or 52 weeks; or
the antibody is sarilumab.

53-58. (canceled)

59. The method of claim 51, wherein:
the subject is at least 50 years olds;
the subject has bilateral shoulder pain;
the subject has a C-reactive protein (CRP) level of >10 mg/L and/or an erythrocyte sedimentation rate (ESR)>30 mm/h;
the subject has morning stiffness;
the subject has an absence of joint involvement other than the shoulder joint;
the subject has hip pain or limited range of motion;
the subject is seronegative for rheumatoid factor (RF) and anti-cyclic citrullinated peptide (anti-CCP);
the subject does not have a disorder or disease selected from the group consisting of giant cell arteritis, rheumatoid arthritis, inflammatory arthritis, connective tissue disease, rhabdomyolysis, neuropathic muscular disease, and active fibromyalgia; or
the subject has at least one shoulder with subdeltoid bursitis and/or biceps tenosynovitis and/or posterior or axillary glenohumeral synovitis, and at least one hip with synovitis and/or trochanteric bursitis.

60-66. (canceled)

67. The method of claim 59, wherein the connective tissue disease is selected from the group consisting of systemic lupus erythematosus, systemic sclerosis, vasculitis, myositis, mixed connective tissue disease, and ankylosing spondylitis.

68. (canceled)

69. The method of claim 51, wherein at least one symptom of polymyalgia rheumatica in the subject is improved after administering the antibody or antigen-binding fragment thereof.

70. The method of claim 69, wherein the symptom is selected from the group consisting of: shoulder pain associated with inflammatory stiffness; hip pain associated with inflammatory stiffness; elevated C-reactive protein (CRP) levels; and elevated erythrocyte sedimentation rate (ESR).

71. The method of claim 51, wherein treatment with the antibody or antigen-binding fragment thereof results in the improvement of least one patient reported outcome measure or clinician reported outcome measure selected from the group consisting of: functional assessment of chronic illness therapy fatigue scale (FACIT-Fatigue), EuroQol five-dimensional three-level questionnaire (EQ-5D-3L), and Short form-36v2 (SF-36v2), health assessment questionnaire disability index (HAQ-DI), and physician global assessment of disease activity-Visual Analog Scale (MD-VAS).

72. The method of claim 51, wherein treatment with the antibody or antigen-binding fragment thereof results in:
a decrease in glucocorticoid toxicity index (GTI) score;
a decrease in PMR activity score (PMR-AS);
an increase in time to first PMR flare;
remission of PMR in the subject; or
an absence of disease flare in remission.

73. (canceled)

74. (canceled)

75. The method of claim 72, wherein the symptoms of PMR flare are selected from the group consisting of shoulder pain associated with inflammatory stiffness and hip girdle pain associated with inflammatory stiffness.

76-80. (canceled)

81. The method of claim 72, wherein:
the remission is achieved at week 12 after starting treatment with the antibody or antigen-binding fragment thereof, or the remission is sustained at week 12, or week 16, or week 24 or week 52 after starting treatment with the antibody or antigen-binding fragment thereof.

82-85. (canceled)

86. The method of claim 51, wherein treatment with the antibody or antigen-binding fragment thereof results in increased resolution of PMR signs and symptoms or GC-free resolution of PMR signs and symptoms in the subject, or the resolution of PMR signs and symptoms was achieved from week 4 after starting treatment with the antibody or antigen-binding fragment thereof, or the GC-free resolution of PMR signs and symptoms is maintained from week 16 after starting treatment with the antibody or antigen-binding fragment thereof to week 52.

87-96. (canceled)

97. A method for treating polymyalgia rheumatica (PMR) in a subject in need thereof comprising administering an effective amount of an antibody or antigen-binding fragment thereof that specifically binds IL-6 receptor, wherein the antibody or antigen-binding fragment thereof comprises heavy chain complementarity determining region (HCDR) sequences of SEQ ID NOs: 3, 4 and 5, and comprises light chain complementarity determining region (LCDR) sequences of SEQ ID NOs: 6, 7 and 8, and wherein the subject has had an inadequate response to steroids or wherein the subject cannot tolerate steroid taper.

98. The method of claim 97, wherein the steroids comprise corticosteroids that optionally comprise prednisone.

99. (canceled)

100. The method of claim 97, wherein the subject is an adult.

101-117. (canceled)

118. The method of claim 1, wherein the antibody or antigen-binding fragment thereof is administered subcutaneously.

119. The method of claim 1, wherein the antibody or antigen-binding fragment is administered subcutaneously using a needle and syringe, a pen delivery device, or an autoinjector.

120. The method of claim 1, wherein the antibody or antigen binding fragment thereof is administered using a prefilled syringe containing about 175 mg/mL sarilumab.

121. The method of claim 97, wherein the subject was previously treated with a dose of ≥7.5 mg/day, and/or ≤25 mg/day or ≤20 mg/day, of prednisone.

122. The method of claim 97, wherein the dose of corticosteroid is discontinued or tapered or discontinued beginning at between about 10 weeks and about 20 weeks after administering a first dose of the antibody, or discontinued beginning at about 14 weeks after administering a first dose of the antibody, or discontinued or tapered to <2.5 or 2.0 mg prednisone/day.

123. The method of claim 97, wherein the antibody is sarilumab.

124. The method of claim 97, wherein the antibody or antigen-binding fragment thereof is administered subcutaneously.

125. The method of claim 97, wherein the antibody or antigen-binding fragment is administered subcutaneously using a needle and syringe, a pen delivery device, or an autoinjector.

126. The method of claim 97, wherein the antibody or antigen binding fragment thereof is administered using a prefilled syringe containing about 175 mg/mL sarilumab.


SEQUENCE LISTING (19 sequences)

SEQ ID NO: 1 - protein, 116 aa, synthetic construct
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSS

SEQ ID NO: 2 - protein, 107 aa, synthetic construct
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIK

SEQ ID NO: 3 - protein, 8 aa, synthetic construct
RFTFDDYA

SEQ ID NO: 4 - protein, 8 aa, synthetic construct
ISWNSGRI

SEQ ID NO: 5 - protein, 9 aa, synthetic construct
AKGRDSFDI

SEQ ID NO: 6 - protein, 6 aa, synthetic construct
QGISSW

SEQ ID NO: 7 - skipped

SEQ ID NO: 8 - protein, 9 aa, synthetic construct
QQANSFPYT

SEQ ID NO: 9 - protein, 446 aa, synthetic construct
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 358 aa, Homo sapiens
MVAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQD

SEQ ID NO: 12 - protein, 214 aa, synthetic construct [sequence omitted]

SEQ ID NO: 13 - protein, 447 aa, synthetic construct
VQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 11 aa, synthetic construct
RASQDISSYLN

SEQ ID NO: 15 - protein, 7 aa, synthetic construct
YTSRLHS

SEQ ID NO: 16 - protein, 9 aa, synthetic construct
QQGNTLPYT

SEQ ID NO: 17 - protein, 6 aa, synthetic construct
SDHAWS

SEQ ID NO: 18 - protein, 15 aa, synthetic construct
YISYSGITTYNPSLK

SEQ ID NO: 19 - protein, 10 aa, synthetic construct
SLARTTAMDY